(19) United States
(12) Patent Application Publication
(10) Pub. No.: US 2020/0123238 A1
(43) Pub. Date: Apr. 23, 2020

(54) EFFICACY OF AN ANTI-C5 ANTIBODY IN THE PREVENTION OF ANTIBODY MEDIATED REJECTION IN SENSITIZED RECIPIENTS OF A KIDNEY TRANSPLANT

(71) Applicant: Alexion Pharmaceuticals, Inc., Boston, MA (US)

(72) Inventor: Camille BEDROSIAN, Woodbridge, CT (US)

(73) Assignee: Alexion Pharmaceuticals, Inc., Boston, MA (US)

(21) Appl. No.: 16/605,441

(22) PCT Filed: Apr. 17, 2018
(86) PCT No.: PCT/US18/27899

§ 371 Date: Oct. 15, 2019

Related U.S. Application Data
(60) Provisional application No. 62/487,175, filed on Apr. 19, 2017.

(51) Int. Cl. C07K 16/18; A61P 37/06; A61K 9/00; A61K 39/395; A61K 31/436; A61K 31/5377; A61K 31/573

(52) U.S. Cl. CPC C07K 16/18; A61P 37/06; A61K 9/0019; A61K 2039/545; A61K 31/436; A61K 31/5377; A61K 31/573; A61K 39/3955

(57) ABSTRACT

This disclosure provides methods for reducing antibody mediated rejection (AMR) in a human kidney transplant recipient, comprising administering a therapeutically effective amount of an anti-C5 antibody, or antigen-binding fragment thereof, to the recipient in a phased dosing schedule following reperfusion of a kidney allograft, wherein the recipient is sensitized to a human living donor and wherein the recipient receives about two or more weeks of desensitization therapy prior to transplantation.

### RELATED APPLICATIONS

This application is a 35 U.S.C. 371 national stage filing of International Application No. PCT/US2018/027899, filed on Apr. 17, 2018, which claims priority and the benefit of U.S. Provisional Patent Application No. 62/487,175 filed on Apr. 19, 2017. The contents of these applications are incorporated herein by reference in their entireties.

### TECHNICAL FIELD

This invention relates to the field of antibody mediated rejection (AMR).

### SEQUENCE LISTING

The instant application contains a Sequence Listing which has been submitted electronically in ASCII format and is hereby incorporated by reference in its entirety. Said ASCII copy, created on Oct. 15, 2019, is named AXJ_239US_SL.txt and is 46749 bytes in size.

### BACKGROUND

Solid organ transplantation remains the most effective form of therapy for treatment of patients with end-stage kidney disease. In 2008, there were over 80,000 patients in the U.S. on the waiting list for kidney transplant; only one fifth of these patients received a transplant. In addition to the shortage of available organs, an impediment to successful kidney transplantation is the number of sensitized recipients.

Nearly a third of potential recipients on the Organ Procurement and Transplantation Network (UNOS) renal transplant waiting-list are considered sensitized (defined as a Panel Reactive Antibody [PRA] score >10%). These patients have pre-formed antibodies against an array of donor-specific human leukocyte antigens (HLA or DSAs). Sensitization can occur from previous exposure to donor antigens through blood transfusions, pregnancy, and/or prior organ transplantation. The presence of DSAs can lead to AMR, with three types being reported: (a) Hyperacute rejection which presents within minutes of revascularization; (b) AMR which presents within days to weeks after transplantation; and (c) Chronic antibody mediated rejection which occurs following the “de novo” generation of donor-specific antibodies and generally occurs several months to years from the time of transplant.

There have been reported early success rates of over 90% at one year following transplant in several centers for newer desensitization approaches for sensitized renal transplant recipients, approaches including intravenous immunoglobulins (IVIg) and plasmapheresis. However, AMR remains an important issue because data suggests that in patients who developed AMR, long-term allograft function and survival are impaired. Hence, the prevention of AMR is critically important in attaining the best possible long-term results in sensitized renal transplant patients.

To date, there are no FDA approved therapeutic agents indicated for the prevention of AMR.

### SUMMARY

This disclosure provides a method for reducing the likelihood that a human kidney transplant recipient sensitized to a living donor will develop antibody mediated rejection.

In certain aspects, the method includes: selecting the living donor; selecting the kidney transplant recipient, the recipient being sensitized to the donor; administering a desensitization therapy to the recipient prior to transplantation; transplanting the kidney from the donor to the recipient; and administering a therapeutically effective dose of an anti-complement C5 antibody, or an antigen binding fragment thereof, to the recipient; the anti-complement C5 antibody, or an antigen binding fragment thereof, being administered prior to reperfusion of the kidney allograft, and post-transplantation in a phased dosing schedule. In some embodiments, the anti-complement C5 antibody is eculizumab.

In one embodiment, methods of reducing antibody mediated rejection (AMR) in a human kidney transplant recipient are provided, comprising administering a therapeutically effective amount of an anti-C5 antibody, or antigen-binding fragment thereof, to the recipient in a phased dosing schedule following reperfusion of a kidney allograft, wherein the recipient is sensitized to a human living donor and wherein the recipient receives about two or more weeks (e.g., three, four, five, six, or seven weeks) of desensitization therapy prior to transplantation.

In one embodiment, the phased dosing schedule comprises about a 1200 mg dose of antibody administered about 1 hour prior to kidney allograft reperfusion; followed by about a 900 mg dose administered at about day 1, and about day 7, and about day 14, and about day 21, and about day 28 post transplantation; and about a 1200 mg dose administered at about week 5; and about week 7, and about week 9 post transplantation.

In one embodiment, the recipient is sensitized to a human living donor based on prior exposure to HLA (e.g., the recipient's medical history includes prior exposure to HLA). In one embodiment, the prior exposure to HLA includes one or more of prior solid organ or tissue allograft, pregnancy, blood transfusion, and/or prior exposure to the specific donor's HLA.

The desensitization therapy includes any suitable regimen. In one embodiment, the the desensitization therapy comprises intravenous immunoglobulin treatment (IVIg). In another embodiment, the desensitization therapy comprises plasmapheresis treatment.

Patients treated according to the methods disclosed herein preferably experience one or more therapeutic improvements. For example, in one embodiment, the recipient experiences reduced AMR compared to standard of care (SOC). In another embodiment, the recipient experiences reduced graft loss compared to SOC. In another embodiment, the recipient experiences a clinically meaningful low level of circulating anti-donor specific antibodies during about the first 9 weeks post transplantation compared to the absence of therapy with the antibody, or antigen binding fragment thereof. In another embodiment, the recipient experiences a clinically meaningful low level of circulating anti-donor specific antibodies during about the first 12 months post transplantation, compared to the absence of therapy with the antibody, or antigen binding fragment thereof. In another embodiment, the recipient experiences clinically meaningful low level of morphologic evidence of acute tissue injury during about the first 9 weeks post transplantation, compared to the absence of therapy with the antibody, or antigen binding fragment thereof. In another embodiment, the recipient experiences clinically meaningful low level of morphologic evidence of acute tissue injury during about the first 12 months post transplantation, compared to the absence of therapy with the antibody or antigen binding fragment thereof. In another embodiment, the recipient experiences a clinically meaningful increase in graft survival at about week 9 post transplantation, compared to the absence of therapy with the antibody or antigen binding fragment thereof. In another embodiment, the recipient experiences a clinically meaningful increase in graft survival at about month 12 post transplantation, compared to the absence of therapy with the antibody or antigen binding fragment thereof. In another embodiment, the recipient experiences increased survival at about 9-weeks post transplantation compared to the absence of therapy with the antibody or antigen binding fragment thereof. In another embodiment, the recipient experiences increased survival at about 12-months post transplantation compared to the absence of therapy with the antibody or antigen binding fragment thereof. In another embodiment, the recipient experiences increased survival at about 36-months post transplantation compared to the absence of therapy with the antibody or antigen binding fragment thereof. In another embodiment, the recipient experiences clinically meaningful low histological evidence of antibody mediated rejection during about the first 9 weeks post transplantation, compared to the absence of therapy with the antibody or antigen binding fragment thereof. In another embodiment, the recipient experiences clinically meaningful low histological evidence of antibody mediated rejection during about the first 12 months post transplantation, compared to the absence of therapy with the antibody, or antigen binding fragment thereof. In another embodiment, the recipient experiences a clinically meaningful low pathological changes, including chronic AMR, on biopsies during about the first 9 weeks post transplantation, compared to the absence of therapy with the antibody or antigen binding fragment thereof. In another embodiment, the recipient experiences a clinically meaningful low pathological changes, including chronic AMR, on biopsies during about the first 12 months post transplantation compared to the absence of therapy with the antibody or antigen binding fragment thereof. In another embodiment, the recipient has reduced need for plasmapheresis treatments during about the first 9 weeks post transplantation compared to the absence of therapy with the antibody, or antigen binding fragment thereof. In another embodiment, the recipient has reduced need for plasmapheresis treatments during about the first 12 months post transplantation compared to the absence of therapy with the antibody, or antigen binding fragment thereof. In another embodiment, the recipient experiences clinically meaningful reduced delayed graft function post transplantation compared to the absence of therapy with the antibody, or antigen binding fragment thereof. In another embodiment, the recipient experiences clinically meaningful reduction in need for dialysis during about the first 9 weeks post transplantation, compared to the absence of therapy with the antibody, or antigen binding fragment thereof. In another embodiment, the recipient experiences a clinically meaningful reduction in need of dialysis during about the first 12 months post transplantation, compared to the absence of therapy with the antibody, or antigen binding fragment thereof. In another embodiment, the recipient experiences stable renal function during about the first 9 weeks post transplantation compared to the absence of therapy with the antibody, or antigen binding fragment thereof. In another embodiment, the recipient experiences stable renal function during about the first 12 months post transplantation compared to the absence of therapy with the antibody, or antigen binding fragment thereof.

In another aspect, methods of reducing antibody mediated rejection (AMR) in a human kidney transplant recipient are provided, comprising administering a therapeutically effective amount of an anti-C5 antibody, or antigen-binding fragment thereof, to the recipient in a phased dosing schedule following reperfusion of a kidney allograft, wherein the recipient is sensitized to a human living donor and wherein the recipient receives about two or more weeks of desensitization therapy prior to transplantation, wherein the recipient experiences during about the first 9 weeks post transplantation, during about the first 12 months post transplantation, and/or during about the first 36 months post transplantation, one or more of: clinically meaningful low level of circulating anti-donor specific antibodies, clinically meaningful low level of morphologic evidence of acute tissue injury, clinically meaningful low histological evidence of antibody mediated rejection, increased greater survival, or increased survival, clinically meaningful low histological evidence of antibody mediated rejection, clinically meaningful low pathological changes, including chronic AMR, on biopsies, reduced need for plasmapheresis treatments, clinically significant reduction in need of dialysis, compared to the absence of therapy with the antibody or antigen binding fragment thereof.

The anti-C5 antibody, or an antigen-binding fragment thereof, can be administered by any suitable means. In one embodiment, the anti-C5 antibody, or an antigen-binding fragment thereof, is administered through intravenous infusion. In another embodiment, the anti-C5 antibody, or an antigen-binding fragment thereof, is administered subcutaneously. In one embodiment, the recipient's plasma levels of anti-C5 antibody, or an antigen binding fragment thereof, is maintained at about 50 to about 100 μg/mL for about the first week post transplantation. In another embodiment, the recipient's plasma levels of anti-C5 antibody, or an antigen binding fragment thereof, is maintained at about 50 to about 100 μg/mL for about the first 9 weeks post transplantation.

The anti-C5 antibody, or antigen binding fragment thereof, can be administered alone or in combination with one more additional therapeutic agents (e.g., two, three, four, or five additional therapeutic agents). In one embodiment, the additional therapeutic agent is an immunosuppressive drug (e.g., tacrolimus, mycophenolate mofetil, and/or prednisone). In one embodiment, no more than three additional therapeutic agents are administered to the patient, in addition to the anti-C5 or anti-C5a antibody, or antigen binding fragment thereof. In another embodiment, no more than two additional therapeutic agents are administered to the patient, in addition to the anti-C5 or anti-C5a antibody, or antigen binding fragment thereof. In another embodiment, no more than one additional therapeutic agent is administered to the patient, in addition to the anti-C5 or anti-C5a antibody, or antigen binding fragment thereof. In another embodiment, no additional therapeutic agents are administered to the patient.

The anti-C5 antibody, or antigen binding fragment thereof, and one more additional therapeutic agents can be administered together or separately. In one embodiment, the anti-C5 or anti-C5a antibody is administered prior to administration of the one more additional therapeutic agents. In another embodiment, the anti-C5 or anti-C5a antibody is administered after administration of the one more additional therapeutic agents. Such concurrent or sequential administration preferably results in the antibody, or antigen binding fragment thereof, and one more additional therapeutic agents being simultaneously present in treated patients.

An exemplary anti-C5 antibody is eculizumab (also known as Soliris®) comprising the heavy and light chains having the sequences shown in SEQ ID NOs:10 and 11, respectively, or antigen binding fragments and variants. In other embodiments, the antibody comprises the heavy and light chain complementarity determining regions (CDRs) or variable regions (VRs) of eculizumab. Accordingly, in one embodiment, the antibody comprises the CDR1, CDR2, and CDR3 domains of the heavy chain variable (VH) region of eculizumab having the sequence shown in SEQ ID NO:7, and the CDR1, CDR2 and CDR3 domains of the light chain variable (VL) region of eculizumab having the sequence shown in SEQ ID NO:8. In another embodiment, the antibody comprises CDR1, CDR2 and CDR3 heavy chain sequences as set forth in SEQ ID NOs:1, 2, and 3, respectively, and CDR1, CDR2 and CDR3 light chain sequences as set forth in SEQ ID NOs:4, 5, and 6, respectively. In another embodiment, the anti-C5 antibody or an antigen binding fragment thereof comprises a heavy chain constant region having the amino acid sequences set forth in SEQ ID NO: 9. In another embodiment, the anti-C5 antibody or an antigen binding fragment thereof comprises the entire heavy chain and light chains having the amino acid sequences set forth in SEQ ID NO: 10 and SEQ ID NO: 11, respectively.

Another exemplary anti-C5 antibody is antibody BNJ441 (also known as ALXN1210) comprising the heavy and light chains having the sequences shown in SEQ ID NOs:14 and 11, respectively, or antigen binding fragments and variants thereof. In other embodiments, the antibody comprises the heavy and light chain complementarity determining regions (CDRs) or variable regions (VRs) of antibody BNJ441. Accordingly, in one embodiment, the antibody comprises the CDR1, CDR2, and CDR3 domains of the heavy chain variable (VH) region of antibody BNJ441 having the sequence shown in SEQ ID NO:12, and the CDR1, CDR2 and CDR3 domains of the light chain variable (VL) region of antibody BNJ441 having the sequence shown in SEQ ID NO:8. In another embodiment, the antibody comprises VH and VL regions having the amino acid sequences set forth in SEQ ID NO:12 and SEQ ID NO:8, respectively. In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively. In another embodiment, the antibody comprises a heavy chain constant region as set forth in SEQ ID NO:13.

In another embodiment, the antibody comprises a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In another embodiment, the antibody comprises CDR1, CDR2 and CDR3 heavy chain sequences as set forth in SEQ ID NOs:19, 18, and 3, respectively, and CDR1, CDR2 and CDR3 light chain sequences as set forth in SEQ ID NOs:4, 5, and 6, respectively and a variant human Fc constant region that binds to human neonatal Fc receptor (FcRn), wherein the variant human Fc CH3 constant region comprises Met-429-Leu and Asn-435-Ser substitutions at residues corresponding to methionine 428 and asparagine 434, each in EU numbering.

In another embodiment, the antibody competes for binding with, and/or binds to the same epitope on C5 as, the above-mentioned antibodies. In another embodiment, the antibody has at least about 90% variable region amino acid sequence identity with the above-mentioned antibodies (e.g., at least about 90%, 95% or 99% variable region identity with SEQ ID NO:7. SEQ ID NO:8, or SEQ ID NO:12).

### BRIEF DESCRIPTION OF THE DRAWING

FIG. 1 illustrates schematically the study design for reducing or preventing AMR in recipients of living donor kidney transplants.

### DETAILED DESCRIPTION

### Definitions

As used herein the term “complement-mediated damage” refers to a pathological condition in which complement activation contributes in an observable or measurable way to the pathology of the condition. For example, complement-mediated damage can be characterized by the destruction of cells through complement activation.

As used herein the term “reducing” refers to a decrease by a statistically significant amount. For example, in one embodiment, reducing refers to either partially or completely inhibiting an activity or decreasing or lowering an activity. In one embodiment, “reducing” means a decrease by at least 10% compared to a reference level, for example a decrease by at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or up to and including a 100% decrease compared to a reference sample, or any decrease between BOUT 10-100% compared to a reference level.

As used herein, the term “reducing the incidence” and “improving function” refer to a beneficial effect, e.g., amelioration or an improvement over baseline. Frequently the improvement over baseline is statistically significant. For example, “reducing the incidence” and “improving function” may refer to an amelioration of at least about 10% as compared to a reference level, for example, an improvement of at least about 20%, or at least about 30%, or at least about 40%, or at least about 50%, or at least about 60%, or at least about 70%, or at least about 80%, or at least about 90% or up to and including a 100% improvement or any improvement between 10-100% as compared to a reference level, or at least about a 2-fold, or at least about a 3-fold, or at least about a 4-fold, or at least about a 5-fold, or at least about a 6-fold, or at least about a 7-fold, or at least about a 8-fold, or at least about a 9-fold, or at least about a 10-fold improvement, or any improvement between about 2-fold and 10-fold or greater, as compared to a reference level.

As used herein “stable renal function” refers to renal function which may be estimated by Glomerular Filtration Rate (calculated) by Modification of Diet in Renal Disease 7 (MDRD7) or serum creatinine. Generally, stable renal function refers to renal function which varies by less than 60%, less than 50%, less than 40%, less than 30%, less than 20%, less than 10%, less than 5%, less than 2%, less than 1%, or less than 0.5%, between repeated measurements of estimated by Glomerular Filtration Rate and serum creatinine. For example, sometimes 1, 2, 3, 4 or more repeated measurements of Glomerular Filtration Rate and/or serum creatinine may be needed to determine whether or not there has been a change in renal function.

As used herein the term “transplant” refers to the replacement of an organ, for example, a kidney, in a human or non-human animal recipient. The purpose of replacement is to remove a diseased organ or tissue in the host and replace it with a healthy organ or tissue from the donor. Where the donor and the recipient are the same species the transplant is known as an “allograft”. Where the donor and the recipient are dissimilar species the transplant is known as a “xenograft”. The techniques necessary for transplantation are varied and depend to a large extent on the nature of the organ being transplanted. The success of the transplant as a therapeutic modality depends on a number of possible physiological outcomes. For example, the host may reject the new organ via antibody-dependent hyperacute rejection mechanisms, cell-mediated acute rejection or chronic degenerative processes.

The term “sensitized” used in connection with a recipient refers to a recipient that has exceptionally high antibody levels that react to foreign tissue, such as a donated organ.

As used herein, the term “desensitization” refers to DSA reduction techniques used to facilitate kidney transplantation for recipients who are sensitized to their donor organs by lowering the amount of circulating DSA. Techniques include, for example, direct antibody removal by plasmapheresis (PP), immune modulation using intravenous immunoglobulins (this term used interchangeably with immune globulins) (IVIg), and attempts to deplete B cells using a variety of immunosuppressive agents.

As used herein, the phrase “plasma exchange” (also known as “plasmapheresis”) is a process by which the liquid portion of part of a patient's blood (plasma) is removed and separated from the blood cells. The basic procedure consists of removal of blood, separation of blood cells from plasma, and return of these blood cells to the body's circulation, diluted with fresh plasma or a substitute. Because of concerns over viral infection and allergic reaction, fresh plasma is not routinely used. Instead, the most common substitute is saline solution with sterilized human albumin protein. During the course of a single session, two to three liters of plasma is removed and replaced. Plasmapheresis requires insertion of a venous catheter, either in a limb or central vein. Central veins allow higher flow rates and are more convenient for repeat procedures, but are more often the site of complications, especially bacterial infection. When blood is outside the body, it must be treated to prevent it from clotting. While most of the anticlotting agent is removed from the blood during treatment, some is returned to the patient. Three procedures are available. The first is “discontinuous flow centrifugation”, wherein only one venous catheter line is required. Approximately 300 ml of blood is removed at a time and centrifuged to separate plasma from blood cells. The second is “continuous flow centrifugation”, wherein two venous lines are used. This method requires slightly less blood volume to be out of the body at any one time. The third procedure is “plasma filtration”, wherein two venous lines are used. The plasma is filtered using standard hemodialysis equipment and it requires less than 100 ml of blood to be outside the body at one time.

As used herein, the term “rejection” refers to the process or processes by which the immune response of an organ transplant recipient mounts a reaction against the transplanted organ, cell or tissue, sufficient to impair or destroy normal function of the organ. The immune system response can involve specific (antibody and T cell-dependent) or non-specific (phagocytic, complement-dependent, etc.) mechanisms, or both.

The term “effective amount” refers to an amount of an agent that provides the desired biological, therapeutic, and/or prophylactic result.

“Dose” refers to an amount of a drug given in a single administration.

Some drugs are dosed according to body weight (mg/kg) or body surface area (BSA) (mg/m²). As used herein, a “body surface area (BSA)-based dose” refers to a dose of an agent that is adjusted to the body-surface area (BSA) of the individual patient. Various calculations have been published to arrive at the BSA without direct measurement, the most widely used of which is the Du Bois formula (see Du Bois D, Du Bois E F (June 1916) Archives of Internal Medicine 17 (6): 863-71; and Verbraecken, J. et al. (April 2006). Metabolism Clinical and Experimental 55 (4): 515-24). Other exemplary BSA formulas include the Mosteller formula (Mosteller R D. N Engl J Med., 1987; 317:1098), the Haycock formula (Haycock G B, et al., J Pediatr 1978, 93:62-66), the Gehan and George formula (Gehan E A, George S L, Cancer Chemother Rep 1970, 54:225-235), the Boyd formula (Current, J D (1998), The Internet Journal of Anesthesiology 2 (2); and Boyd, Edith (1935), University of Minnesota. The Institute of Child Welfare, Monograph Series, No. x. London: Oxford University Press), the Fujimoto formula (Fujimoto S, et al., Nippon Eiseigaku Zasshi 1968; 5:443-50), the Takahira formula (Fujimoto S, et al., Nippon Eiseigaku Zasshi 1968; 5:443-50), and the Schlich formula (Schlich E, et al., Ernahrungs Umschau 2010; 57:178-183).

Other drugs are dosed according to a “fixed dose.” As used herein, the terms “fixed dose”, “flat dose” and “flat-fixed dose” are used interchangeably and refer to a dose that is administered to a patient without regard for the weight or body surface area (BSA) of the patient. The fixed or flat dose is therefore, not provided as a mg/kg dose, but rather as an absolute amount of the agent (e.g., the anti-C5 antibody, or antigen binding fragment thereof).

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly 0 materials are described herein for use in the present invention; other, suitable methods and materials known in the art can also be used.

As used herein, the terms “subject” or “patient” are used interchangeably and include a human patient. A recipient or a donor is a subject.

As used herein, the terms “about two or more weeks of desensitization therapy” generally refers to a recipient receiving about 11, 12, 13, 14, 15, 16, 17, 18, 19, 20, 21, 22, 23, 24, 25, 26, 27, 28, 29, 30, or 31, days of therapy, or about 2 to about 3, about 3 to about 4, about 2 to about 4, about 1.5 to 2.5, about 2.5 to about 3.5 or about 3.5 to about 4.5 weeks of therapy. Likewise, “about 2 weeks” may refer to about 11, 12, 13, 14, 15, 16, or 17 days, or about 1.5 to about 2.5 weeks; “about 3 weeks” may refer to about 18, 19, 20, 21, 22, 23, or 24 days, or about 2.5 to about 3.5 weeks; and “about 4 weeks” may refer to about 25, 26, 27, 28, 29, 30, or 31 days, or about 3.5 to about 4.5 weeks.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Antibody Mediated Rejection (AMR)

Antibody mediated rejection (AMR), a rejection reaction that results from the action of antibodies on the allograft, is a significant obstacle to successful kidney transplantation. This form of rejection causes severe and rapid dysfunction and loss of allografts. Takemoto S K et al. Am J Transplant. 2004 July; 4(7):1033-41; McKenna R M et al. Transplantation. 2000 Feb. 15; 69(3):319-26; Feucht H E, Opelz G. Kidney Int. 1996 November; 50(5):1464-75; and Mauiyyedi S et al. J Am Soc Nephrol. 2002 March; 13(3):779-87.

The most common mechanism underlying AMR is an anamnestic response originating from previous antigenic exposure. These donor specific antibody (DSA) responses are usually robust and result in the rapid production of high levels of DSA and acute allograft dysfunction. Singh N, Pirsch J, Samaniego M. Transplant Rev (Orlando). 2009 January; 23(1):34-46. The mechanism of injury in AMR involves antigens that initiate the production of DSAs resulting in antigen-antibody interactions, complement activation and inflammation, and the resultant donor tissue damage. Trpkov K et al., Transplantation. 1996 Jun. 15; 61(11):1586-92.

The main target of DSA's is endothelial cells within the microcirculation of the donor organ. This leads to activation of the complement cascade which initiates injury to the capillaries. Complement activation leads to C4d deposition in the peritubular capillaries of the renal allograft. Collins A B et al. J Am Soc Nephrol. 1999 October; 10(10):2208-14; Halloran P F. Am J Transplant. 2003 June; 3(6):639-40. This C4d deposition is an important diagnostic criterion for the development of AMR.

The impact of AMR on graft survival is dramatic and continues long after the initial inflammatory condition has resolved as was recently demonstrated in a study by LeFaucheur and Glotz. In this single center study of a large cohort of sensitized recipients, the investigators compared allograft survival for recipients successfully treated for AMR versus those that never experienced AMR. Lefaucheur and Glotz Trends in Transplant 4. 2010, 4; pp. 3-10.

The effect of AMR on allograft survival, in spite of successful AMR treatment, is demonstrated by the data in Table 1. The data in this single center study of deceased donor kidney recipients, who were sensitized to their donors, compared the survival of the transplanted kidneys for those who experienced AMR to those who did not. The outcomes were independent of whether the recipients continued to have persistent DSA. The results strongly support the concept that prevention of the inflammatory lesion of AMR, rather than treatment intervention once AMR develops, is the key factor to transplantation across the humoral immune barrier. Lefaucheur and Glotz Trends in Transplant 4. 2010, 4 pp. 3-10. All but two episodes of AMR occurred within six weeks, with most occurring within four weeks of transplantation, which is consistent with multiple reports in the literature by numerous investigators describing AMR as a very early clinical event. Montgomery R A, Zachary A A. Pediatr Transplant. 2004 December; 8(6):535-42; Thielke J J et al. Transplantation. 2009 Jan. 27; 87(2):268-73; Truong L D et al., Arch Pathol Lab Med. 2007 August; 131(8): 1200-8.

TABLE 1
Allograft Survival for DSA+ DD Kidney Transplant
Recipients With and Without AMR

Recipient
Allograft
Survival with and
without AMR

| | AMR+ | AMR− |
|---|---|---|
| Time Point | N = 29 | N = 54 |
| 1 year | 79.3% | 88.6% |
| 3 years | 68.9% | 88.6% |
| 8 years | 41.7% | 71.8% |

The key results from these additional reports are summarized in Table 2. Stegall, et al. described a series of 19 kidney transplant recipients who received kidney transplants following desensitization and who developed AMR. All occurrences of AMR occurred within the first six weeks and most within four weeks post transplantation. Stegall M D et al, Am J Transplant. 2006 February; 6(2):346-51. Montgomery and colleagues described another series of 62 patients in whom all instances of AMR occurred within the first 10 days post transplantation. Montgomery R A, Zachary A A. Pediatr Transplant. 2004 December; 8(6):535-42. Regardless of the clinical setting, a common theme is that most instances of AMR are reported to occur very early following transplantation.

TABLE 2
Publications on AMR in Kidney Transplantation

| | | Time to |
|---|---|---|
| | | Diagnosis |
| Author/year (reference) | Number of Patients | of AMR |
| Stegall (2006) Stegall MD et | 19 | <6 weeks |
| al, Am J Transplant. 2006 | | |
| Feb; 6(2): 346-51 | | |
| Montgomery (2004) | 62 (pediatric population) | <10 days |
| Montgomery RA, Zachary | | |
| AA. Pediatr Transplant. | | |
| 2004 Dec; 8(6): 535-42 | | |
| Rostaing (2009) Rostaing L, | 22 | Mean 21 days |
| et al. Transplantation. 2009 | | |
| Apr 27; 87(8):1261 | | |
| Faguer (2007) Faguer S et al | 8 | <6 weeks |
| Transplantation 2007 May | | |
| 15; 83(9): 1277-80 | | |
| Crespo (2001) Crespo M et | 18 | <22 days |
| al. Transplantation 2001 Mar | | |
| 15; 71(5): 652-8 | | |
| White (2004) White NB et al. | 9 | <28 days |
| Transplantation 2004 Sep | | |
| 15; 78(5): 772-4 | | |
| Braun (2004) Braun N et al., | 1 | Day 7 |
| Transpl Int. 2004 | | |
| Aug; 17(7): 384-6 | | |
| Han (2008) Han DJ et al., | 13 | <10 days |
| Abstract 526. | | |
| Transplantation | | |
| 2008; 86(2S): 184 | | |
| Muro (2005) Muro M et al. | 1 | Day 2 |
| Nephrol Dial Transplant | | |
| 2005 Jan; 20(1): 223-6 | | |
| LeFaucheur (2010) | 29 | <6 weeks |
| Lefaucheur and Glotz Trends | | |
| in Transplant 4. 2010, 4; | | |
| pp. 3-10 | | |
| Higgins (2009) Higgins R et | 36 | <40 days |
| al. Nephrol Dial Transplant | | |
| 2009 Apr; 25(4): 1306-12 | | |

These data demonstrate that AMR is a lesion that occurs early after transplantation and points to the importance of prevention of the acute inflammatory lesion of AMR during the first month post-transplantation.

### Desensitization Protocols, Prophylaxis and Treatment for AMR

DSA reduction techniques (desensitization) are used to facilitate kidney transplantation for recipients who are sensitized to their donor organs by lowering the amount of circulating DSA. DSA reduction techniques to facilitate sensitized living donor transplants continue to evolve. Extensive review of the literature reveals an array of techniques that include direct antibody removal using plasmapheresis (PP), immune modulation using intravenous immune globulins (IVIg), and attempts to deplete B cells using a variety of immunosuppressive agents. Plasmapheresis and IVIg have been well studied and both have been shown to be effective in clinical trials. This observation is supported by a recent report in which Jordan and Pescovitz compared plasmapheresis and IVIg for pre-transplant desensitization as used by three experienced Transplant Centers in the US. These groups use plasmapheresis (plasma exchange) with or without the addition of IVIg therapy. Jordan S C, Pescovitz M D. Clin J Am Soc Nephrol. 2006 May; 1(3):421-32.

### Role of the Complement System in AMR

AMR can result from uncontrolled complement mediated injury, initiated when DSA binds to receptors on the donor organ blood vessel endothelium. This antibody-antigen interaction results in activation of the complement cascade with the resultant production of complement split products C5a and C5b. C5a is a potent anaphylotoxin and inflammatory mediator while C5b is a necessary component for formation of the C5b-9 terminal complement complex, also known as the membrane attack complex. C5b-9 is an activator of leukocytes and vascular cells and stimulates the secretion of mediators from storage granules and the translocation of P-selectin from platelet α-granules to the plasma membrane. P-selectin initiates adhesion of monocytes and platelets to the vascular endothelium and serves as a co-stimulatory molecule for the production of inflammatory mediators. In addition, C5b-9-activated endothelial cells synthesize IL-8, tissue factor and monocyte chemotactic protein 1 (MCP-1), which is an important chemotactic factor in macrophage recruitment to sites of tissue injury.

Complement activation can be documented by measuring complement protein by-products. While some complement components bind to the antibody-antigen complex, others can be found in the local environment. For example, C4d, a stable complement component of the proximal portion of the complement cascade, can be localized by immunohistologic techniques in tissue specimens near sites of inflammation and is used as a marker for complement activation in allograft biopsy specimens.

### HLA Antigens

HLA molecules are membrane bound glycoproteins that bind processed antigenic peptides and present them to T cells. The essential role of the HLA antigens lies in the control of self-recognition and thus defense against microorganisms. Based on the structure of the antigens produced and their function, there are two classes of HLA antigens, HLA Class I and Class II.

HLA Class I antigens are expressed on all nucleated cells of the body. Additionally, they are found in soluble form in plasma and adsorbed onto the surface of platelets. Erythrocytes also adsorb HLA Class I antigens.

The tissue distribution of HLA Class II antigens is confined to the “immune competent” cells, including B-lymphocytes, macrophages, endothelial cells and activated T-lymphocytes. The expression of HLA Class II on cells that would not normally express them is stimulated by cytokines like interferon-γ and is associated with acute graft rejection in the setting of transplantation.

There are important differences in HLA expression between T and B cells, which influence the interpretation of the crossmatch. T cells do not constitutively express HLA class II; so the result of a T-cell crossmatch generally reflects antibodies to HLA class I only. B cells express both HLA class I and II. Because of this, a positive B-cell crossmatch is more difficult to interpret than a positive T-cell crossmatch. It may be due to antibodies directed against HLA class I, II, or both. A negative B-cell crossmatch in the presence of a positive T-cell cross match suggests a technical error. Transplanting in the setting of a positive T-cell crossmatch, which is not due to an autoantibody, is likely to generate a very poor outcome.

B-cell CDC cross matching is not as predictive of hyper acute rejection (HAR) as the T-cell CDC crossmatch. B-cell crossmatches are often performed as part of the immunologic assessment before live donor transplantation when there is more time to determine the significance of the result. Paired with information about the presence of DSA, determined by more specific means such as antigen-coated beads (Luminex, discussed below) the B-cell CDC crossmatch results may be more meaningful. If a B-cell crossmatch is positive and there are no detectable antibodies to class I or II antigens, the result may be falsely positive while a positive result in the presence of detectable DSA signifies that the identified DSA may be functionally relevant in that it can activate complement, and were associated with increased risk of rejection.

### Cross-Matching Techniques

Cross-matching was developed in an attempt to identify recipients who are likely to develop acute vascular rejection of a graft from a given donor. This phenomenon, HAR, is a result of preformed antibodies against the donor; referred to as donor-specific antibodies (DSA). Such antibodies are usually formed as the result of previous exposure to HLA, generally through pregnancy, blood transfusion or previous transplantation.

Preformed antibodies cause rejection by binding to HLA antigens expressed on the endothelium of vessels in the transplanted kidney, resulting in activation of the complement cascade with resultant thrombosis and infarction of the graft.

### Complement-Dependent Cytotoxicity (CDC) Crossmatch

A CDC crossmatch involves placing recipient serum (potentially containing donor-specific anti-HLA antibodies) onto donor lymphocytes (containing HLA antigens). A cytotoxic reaction (deemed ‘positive’) suggests the presence of preformed DSA.

The read-out of the test is the percentage of dead cells relative to live cells as determined by microscopy. The result can thus be scored on the percentage of dead cells, with 0 correlating to no dead cells; scores of 2, 4 and 6 represent increasing levels of lysis. On this basis, a score of 2 is positive at a low level, consistent with approximately 20% lysis (generally taken as the cut-off for a positive result). A score of 8 represents all cells having lysed and indicates the strongest possible reaction. The use of a scoring system allows a semi-quantitative analysis of the strength of reaction. Another way to determine the strength of the reaction is to repeat the crossmatch using serial doubling dilutions of the recipient serum (often known as a ‘titred crossmatch’). In this way, dilutions are usually performed to 1 in 2, 4, 8, 16, 32, 64, etc.

### The Flow Crossmatching Technique

A flow crossmatch involves using the same initial base ingredients as CDC crossmatching (i.e. donor lymphocytes and recipient serum). The two are mixed and then incubating them with fluorescein-labelled antibodies against human IgG (antihuman IgG fluorescein isothicyanate [FITC]). This fluorescein-labelled antibody will bind to all the IgG antibodies in the recipient serum. If a DSA in this serum then binds to the donor lymphocytes, it will be detectable by flow cytometry.

The read-out may be reported simply as positive or negative or can be further quantitated. Intensity of fluorescence above control, referred to as channel shifts, may be reported. Generally, a mean channel shift above 50 indicates that antibody is present and above 150 indicates a very high risk and a contraindication to renal transplant except in exceptional circumstances. Channel shifts above 300 usually correlate with a positive cytotoxic crossmatch.

### Luminex Testing

Luminex testing offers significant advantages over CDC and flow crossmatch in terms of defining the HLA specificity of identified antibodies. The presence of a DSA detected by Luminex in the setting of a negative or positive CDC crossmatch appears to have prognostic importance in terms of graft survival and acute rejection risk; however, there are insufficient data to determine the significance of a DSA with a negative flow crossmatch.

Positive results can then be graded as weak, moderate or strong on the basis of the degree of fluorescence of the Luminex bead array. This result can be scored as a median fluorescence index (MFI). However, Luminex bead array assays are approved only for qualitative assignment of HLA. MFI cannot directly be converted into antibody titers as the MFI simply represents a marker for the bound antibody and is affected by several factors, including antibody concentration in the serum, conformation and orientation of the antigen, and antibody avidity toward the respective antigen.

### Glomerular Filtration Rate

The Glomerular filtration rate (GFR) is a test used to measure how well the kidneys are working. Specifically, it estimates how much blood passes through the glomeruli each minute. Glomeruli are the tiny filters in the kidneys that filter waste from the blood. The GFR may be used to determine a patient's stage of kidney disease.

GFR is equal to the clearance rate when any solute is freely filtered and is neither reabsorbed nor secreted by the kidneys. The rate therefore measured is the quantity of the substance in the urine that originated from a calculable volume of blood. The GFR can be

$$GFR = \frac{( Urine Concentration ) \times ( Urine Flow )}{( Plasma Concentration )} .$$

The product of urine concentration and urine flow equals the mass of substance excreted during the time that urine has been collected. Dividing this mass by the plasma concentration gives the volume of plasma that the mass must have originally come from during the aforementioned period of time. The GFR is typically recorded in units of volume per time, e.g., milliliters per minute mL/min.

The estimated Glomerular filtration rate (eGFR) is used to screen for and detect early kidney damage and to monitor kidney status. It is performed by ordering a creatinine test and calculating the estimated glomerular filtration rate.

The eGFR may be calculated from serum creatine using the Chronic Kidney Disease Epidemiology Collaboration (CKD-EPI) equation.

The CKD-EPI equation, expressed as a single equation, is:

eGFR=141×min(Scr/κ,1)α×max(Scr/κ,1)−1.209×0.993Age×1.018 [if female]×1.159 [if African American].

Where Scr is serum creatinine (mg/dL), κ is 0.7 for females and 0.9 for males, α is −0.329 for females and −0.411 for males, min indicates the minimum of Scr/κ or 1, and max indicates the maximum of Scr/κ or 1.

Alternatively, the estimated glomerular filtration rate may be calculated using the Modification of Diet in Renal Disease (MDRD) 7 Calculation described in Poge U et al., Am J Transplant. 2005 June; 5(6):1306-11 and presented below.

MDRD 7 equation (MDRD7)=170×[serum creatinine(mg/dL)]−0.999×[age]−0.176×[0.762 if patient is female]×[1.18 if patient is black]×[serum urea nitrogen concentration (mg/dL)]−0.170×[serum albumin concentration (g/dL)]0.318.

A person's GFR or eGFR should be interpreted in relation to the person's clinical history and presenting conditions, utilizing Table 3.

TABLE 3
GFR and Kidney Damage

| KIDNEY | | | |
|---|---|---|---|
| DAMAGE | | | |
| STAGE | DESCRIPTION | GFR | OTHER FINDINGS |
| 1 | Normal or minimal | 90+ | Protein or albumin in |
| | kidney damage with | | urine are high, cells |
| | normal GFR | | or casts seen in urine |
| 2 | Mild decrease in GFR | 60-89 | Protein or albumin in |
| | | | urine are high, cells |
| | | | or casts seen in urine |
| 3 | Moderate decrease in | 0-59 | |
| | GFR | | |
| 4 | Severe decrease in GFR | 5-29 | |
| 5 | Kidney failure | 15 | |

### Banff Classification of Rejection

The Banff classification characterizes five categories of renal allograft pathology: (1) AMR; (2) suspicious of acute rejection; (3) acute rejection; (4) chronic sclerosing allograft nephropathy; and (5) other—changes not considered due to rejection.

The diagnosis of AMR in renal allografts is currently based on criteria established during the Banff conference on Allograft Pathology in 2007 (see Table 19), which include the three following cardinal features: (1) morphologic evidence of acute or chronic tissue injury; (2) immunopathological staining for C4d in peritubular capillaries; and (3) presence of circulating antibodies to donor human lymphocyte antigen or other antigens expressed on donor endothelial cells.

It is recommended that every renal allograft biopsy should be stained for C4d. C4d staining is considered positive only when depositions are found in the peritubular capillaries. C4d is scored semi-quantitatively in four categories:

(1) No C4d staining (0% of (peritubular) capillaries)

(2) Minimal C4d staining (0-10% of (peritubular) capillaries)

(3) Focal C4d staining (10-50% of (peritubular) capillaries)

(4) Diffuse C4d staining (>50% of (peritubular) capillaries).

### Anti-C5 Antibodies

The term “antibody” describes polypeptides comprising at least one antibody derived antigen binding site (e.g., VH/VL region or Fv, or CDR). Antibodies include known forms of antibodies. For example, the antibody can be a human antibody, a humanized antibody, a bispecific antibody, or a chimeric antibody. The antibody also can be a Fab, Fab′2, ScFv, SMIP, Affibody®, nanobody, or a domain antibody. The antibody also can be of any of the following isotypes: IgG1, IgG2, IgG3, IgG4, IgM, IgA1, IgA2, IgAsec, IgD, and IgE. The antibody may be a naturally occurring antibody or may be an antibody that has been altered (e.g., by mutation, deletion, substitution, conjugation to a non-antibody moiety). For example, an antibody may include one or more variant amino acids (compared to a naturally occurring antibody) which changes a property (e.g., a functional property) of the antibody. For example, numerous such alterations are known in the art which affect, e.g., half-life, effector function, and/or immune responses to the antibody in a patient. The term antibody also includes artificial polypeptide constructs which comprise at least one antibody-derived antigen binding site.

The anti-C5 antibodies described herein bind to complement component C5 (e.g., human C5) and inhibit the cleavage of C5 into fragments C5a and C5b. Anti-C5 antibodies (or V_{H}/V_{L} domains derived therefrom) suitable for use in the methods disclosed herein can be generated using methods well known in the art. Alternatively, art recognized anti-C5 antibodies can be used. Antibodies that compete with any of these art-recognized antibodies for binding to C5 also can be used.

An exemplary anti-C5 antibody is eculizumab comprising heavy and light chains having the sequences shown in SEQ ID NOs: 10 and 11, respectively, or antigen binding fragments and variants thereof. Eculizumab (also known as Soliris®) is described in U.S. Pat. No. 6,355,245.

Eculizumab (h5G1.1-mAb solution for infusion) is a humanized monoclonal antibody with binding specificity uniquely specific for the human complement C5 protein. Comprised of 1324 amino acids with a molecular mass of approximately 148 kDa, eculizumab was derived from a murine monoclonal antibody (m5G1.1-mAb) that recognizes the human complement component C5. Humanization of the antibody was achieved by grafting the murine antibody's complementarity determining regions (CDRs) into human antibody-derived variable heavy and light chain framework regions. The constant regions of h5G1.1-mAb include the human kappa light chain and a hybrid IgG human heavy chain. The heavy chain CH₁ domain, hinge region and the first 29 amino acids of the CH₂ domain were derived from human IgG₂, while the remainder of the CH₂ domain and the CH₃ domain originated from human IgG4. Approved by the FDA and European Medicines Agency (EMA) for the treatment of paroxysmal nocturnal hemoglobinuria and atypical hemolytic uremic syndrome, eculizumab is also being studied in other complement-mediated disorders. Hillmen P et al. N Engl J Med. 2006 Sep. 21; 355(12):1233-43; Richards S J et al. Cytometry B Clin Cytom 2007 September; 72(5):291-8; Nurnberger J et al. N Engl J Med. 2009 Jan. 29; 360(5):542-4.

In other embodiments, the antibody comprises the heavy and light chain CDRs or variable regions of eculizumab. Accordingly, in one embodiment, the antibody comprises the CDR1, CDR2, and CDR3 domains of the VH region of eculizumab having the sequence set forth in SEQ ID NO: 7, and the CDR1, CDR2 and CDR3 domains of the V_{L} region of eculizumab having the sequence set forth in SEQ ID NO: 8. In another embodiment, the antibody comprises heavy chain CDR1, CDR2 and CDR3 domains having the sequences set forth in SEQ ID NOs: 1, 2, and 3, respectively, and light chain CDR1, CDR2 and CDR3 domains having the sequences set forth in SEQ ID NOs: 4, 5, and 6, respectively. In another embodiment, the antibody comprises V_{H} and V_{L} regions having the amino acid sequences set forth in SEQ ID NO: 7 and SEQ ID NO: 8, respectively.

Another exemplary anti-C5 antibody is antibody BNJ441 comprising heavy and light chains having the sequences shown in SEQ ID NOs:14 and 11, respectively, or antigen binding fragments and variants thereof. BNJ441 (also known as ALXN1210) is described in PCT/US2015/019225 and U.S. Pat. No. 9,079,949. BNJ441 is a humanized monoclonal antibody that is structurally related to eculizumab (Soliris®). BNJ441 selectively binds to human complement protein C5, inhibiting its cleavage to C5a and C5b during complement activation. This inhibition prevents the release of the proinflammatory mediator C5a and the formation of the cytolytic pore-forming membrane attack complex C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of BNJ441. Accordingly, in one embodiment, the antibody comprises the CDR1, CDR2, and CDR3 domains of the V_{H} region of BNJ441 having the sequence set forth in SEQ ID NO:12, and the CDR1, CDR2 and CDR3 domains of the V_{L} region of BNJ441 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises VH and VL regions having the amino acid sequences set forth in SEQ ID NO:12 and SEQ ID NO:8, respectively. In another embodiment, the antibody may comprise the heavy chain constant region of BNJ441 having the amino acid sequences set forth in SEQ ID NO: 13.

Another exemplary anti-C5 antibody is antibody BNJ421 comprising heavy and light chains having the sequences shown in SEQ ID NOs:10 and 11, respectively, or antigen binding fragments and variants thereof.

Another exemplary anti-C5 antibody comprises heavy and light chains having the sequences shown in SEQ ID NOs:20 and 11, respectively, or antigen binding fragments and variants thereof.

In other embodiments, the antibody comprises the heavy and light chain CDRs or variable regions of BNJ421. Accordingly, in one embodiment, the antibody comprises the CDR1, CDR2, and CDR3 domains of the V_{H} region of BNJ421 having the sequence set forth in SEQ ID NO:12, and the CDR1, CDR2 and CDR3 domains of the V_{L} region of BNJ421 having the sequence set forth in SEQ ID NO:8. In another embodiment, the antibody comprises heavy chain CDR1, CDR2 and CDR3 domains having the sequences set forth in SEQ ID NOs:19, 18, and 3, respectively, and light chain CDR1, CDR2 and CDR3 domains having the sequences set forth in SEQ ID NOs:4, 5, and 6, respectively. In another embodiment, the antibody comprises V_{H} and V_{L} regions having the amino acid sequences set forth in SEQ ID NO:12 and SEQ ID NO:8, respectively. In another embodiment, the antibody may comprise the heavy chain constant region of BNJ421 having the amino acid sequences set forth in SEQ ID NO: 9.

Another exemplary anti-C5 antibody is the 7086 antibody described in U.S. Pat. Nos. 8,241,628 and 8,883,158. In one embodiment, the antibody may comprise the heavy and light chain CDRs or variable regions of the 7086 antibody. In another embodiment, the antibody, or a fragment thereof may comprise comprising heavy chain CDR1, CDR2 and CDR3 domains having the sequences set forth in SEQ ID NOs:21, 22, and 23, respectively, and light chain CDR1, CDR2 and CDR3 domains having the sequences set forth in SEQ ID NOs:24, 25, and 26, respectively. In another embodiment, the antibody or fragment thereof may comprise the V_{H} region of the 7086 antibody having the sequence set forth in SEQ ID NO:27, and the V_{L} region of the 7086 antibody having the sequence set forth in SEQ ID NO:28.

Another exemplary anti-C5 antibody is the 8110 antibody also described in U.S. Pat. Nos. 8,241,628 and 8,883,158. In one embodiment, the antibody may comprise the heavy and light chain CDRs or variable regions of the 8110 antibody. The antibody, or fragment thereof may comprise heavy chain CDR1, CDR2 and CDR3 domains having the sequences set forth in SEQ ID NOs:29, 30, and 31, respectively, and light chain CDR1, CDR2 and CDR3 domains having the sequences set forth in SEQ ID NOs:32, 33, and 34, respectively. In another embodiment, the antibody may comprise the V_{H} region of the 8110 antibody having the sequence set forth in SEQ ID NO:35, and the V_{L} region of the 8110 antibody having the sequence set forth in SEQ ID NO:36.

Another exemplary anti-C5 antibody is SKY59 as described by Fukuzawa et al., Sci Rep. 2017 Apr. 24; 7(1):1080. In one embodiment, the antibody may comprise the heavy and light chain CDRs or variable regions of the SKY59 antibody. In another embodiment, the antibody may comprise heavy and light chains have the sequences set forth in SEQ ID NOs: 37 and 38, respectively.

The exact boundaries of CDRs have been defined differently according to different methods. In some embodiments, the positions of the CDRs or framework regions within a light or heavy chain variable domain can be as defined by Kabat et al. [(1991) “Sequences of Proteins of Immunological Interest.” NIH Publication No. 91-3242, U.S. Department of Health and Human Services, Bethesda, Md.]. In such cases, the CDRs can be referred to as “Kabat C DRs” (e.g., “Kabat LCDR2” or “Kabat HCDR1”). In some embodiments, the positions of the CDRs of a light or heavy chain variable region can be as defined by et al. (1989) Nature 342:877-883. Accordingly, these regions can be referred to as “Chothia CDRs” (e.g., “Chothia LCDR2” or “Chothia HCDR3”). In some embodiments, the positions of the CDRs of the light and heavy chain variable regions can be as defined by a Kabat-Chothia combined definition. In such embodiments, these regions can be referred to as “combined Kabat-Chothia CDRs”. Thomas et al. [(1996) Mol Immunol 33(17/18): 1389-1401] exemplifies the identification of CDR boundaries according to Kabat and Chothia definitions.

Methods for determining whether an antibody binds to a protein antigen and/or the affinity for an antibody to a protein antigen are known in the art. For example, the binding of an antibody to a protein antigen can be detected and/or quantified using a variety of techniques such as, but not limited to, Western blot, dot blot, surface plasmon resonance (SPR) method (e.g., BIAcore system; Pharmacia Biosensor AB, Uppsala, Sweden and Piscataway, N.J.), or enzyme-linked immunosorbent assay (ELISA). See, e.g., Benny K. C. Lo (2004) “Antibody Engineering: Methods and Protocols,” Humana Press (ISBN: 1588290921); Johne et al. (1993) J Immunol Meth 160:191-198; Jonsson et al. (1993) Ann Biol Clin 51:19-26; and Jonsson et al. (1991) Biotechniques 11:620-627.

In one embodiment, the antibody competes for binding with, and/or binds to the same epitope on C5 as, the antibodies described herein. The term “binds to the same epitope” with reference to two or more antibodies means that the antibodies bind to the same segment of amino acid residues, as determined by a given method. Techniques for determining whether antibodies bind to the “same epitope on C5” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to peptide antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same V_{H} and V_{L} or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competing antibodies bind to the same epitope, an overlapping epitope or to adjacent epitopes (e.g., as evidenced by steric hindrance). Anti-C5 antibodies, or antigen-binding fragments thereof described herein, used in the methods described herein can be generated using a variety of art-recognized techniques.

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6: 511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or an antigen binding fragment thereof by screening a DNA library from human B cells according to the general protocol outlined by Huse, et al., Science 246: 1275-1281 (1989).

Any antibody against human C5 (including any kind of antibody), antigen binding fragments and variants thereof, proteins comprising such antibody or fragment, are within the scope of this disclosure.

The term “antibody” is known in the art. The term “antibody” is sometimes used interchangeably with the term “immunoglobulin.” Briefly, it can refer to a whole antibody comprising two light chain polypeptides and two heavy chain polypeptides. Whole antibodies include different antibody isotypes including IgM, IgG, IgA, IgD, and IgE antibodies. The term “antibody” includes, for example, a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

The antibody can also be an engineered protein or antibody-like protein containing at least one immunoglobulin domain (e.g., a fusion protein). The engineered protein or antibody-like protein can also be a bi-specific antibody or a tri-specific antibody, or a dimer, trimer, or multimer antibody, or a diabody, a DVD-Ig, a CODV-Ig, an Affibody®, or a Nanobody®.

The term “antibody fragment,” “antigen-binding fragment,” or similar terms are known in the art and can, for example, refer to a fragment of an antibody that retains the ability to bind to a target antigen (e.g., human C5) and inhibit the activity of the target antigen. Such fragments include, e.g., a single chain antibody, a single chain Fv fragment (scFv), an Fd fragment, a Fab fragment, a Fab′ fragment, or an F(ab′)2 fragment. A scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein. See, e.g., Todorovska et al. (2001) J Immunol Methods 248(1):47-66; Hudson and Kortt (1999) J Immunol Methods 231(1):177-189; Poljak (1994) Structure 2(12):1121-1123; Rondon and Marasco (1997) Annual Review of Microbiology 51:257-283. An antigen-binding fragment can also include the variable region of a heavy chain polypeptide and the variable region of a light chain polypeptide. An antigen-binding fragment can thus comprise the CDRs of the light chain and heavy chain polypeptide of an antibody.

The term “antibody fragment” also can include, e.g., single domain antibodies such as camelized single domain antibodies. See, e.g., Muyldermans et al. (2001) Trends Biochem Sci 26:230-235; Nuttall et al. (2000) Curr Pharm Biotech 1:253-263; Reichmann et al. (1999) J Immunol Meth 231:25-38; PCT application publication nos. WO 94/04678 and WO 94/25591; and U.S. Pat. No. 6,005,079. The term “antibody fragment” also includes single domain antibodies comprising two V_{H} domains with modifications such that single domain antibodies are formed.

### Protocols

This disclosure provides methods of reducing antibody mediated rejection (AMR) in a human kidney transplant recipient, comprising administering a therapeutically effective amount of an anti-C5 antibody, or antigen-binding fragment thereof, to the recipient in a phased dosing schedule following reperfusion of a kidney allograft, wherein the recipient is sensitized to a human living donor and wherein the recipient receives about two or more weeks of desensitization therapy prior to transplantation.

This disclosure also provides methods of reducing antibody mediated rejection (AMR) in a human kidney transplant recipient, comprising administering a therapeutically effective amount of an anti-C5 antibody, or antigen-binding fragment thereof, to the recipient in a phased dosing schedule following reperfusion of a kidney allograft, wherein the recipient is sensitized to a human living donor and wherein the recipient receives about two or more weeks of desensitization therapy prior to transplantation, wherein the recipient experiences during about the first 9 weeks post transplantation, during about the first 12 months post transplantation, and/or during about the first 36 months post transplantation, one or more of: clinically meaningful low level of circulating anti-donor specific antibodies, clinically meaningful low level of morphologic evidence of acute tissue injury, clinically meaningful low histological evidence of antibody mediated rejection, increased greater survival, or increased survival, clinically meaningful low histological evidence of antibody mediated rejection, clinically meaningful low pathological changes, including chronic AMR, on biopsies, reduced need for plasmapheresis treatments, clinically significant reduction in need of dialysis, compared to the absence of therapy with the antibody or antigen binding fragment thereof.

This disclosure also provides methods comprising administering to a subject a pharmaceutical composition comprising the anti-C5 antibody, or an antigen binding fragment thereof, at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In certain embodiments, the method comprises: selecting the living donor; selecting the kidney transplant recipient, wherein the recipient is sensitized to the donor; administering a desensitization therapy to the recipient prior to transplantation; transplanting the kidney from the donor to the recipient; and administering a therapeutically effective dose of an anti-C5 antibody, or an antigen binding fragment thereof, to the recipient.

Both the donor and the recipient may be a human.

In certain embodiments, the recipient's medical history includes at least one of the following sensitizing event: prior solid organ or tissue allograft; pregnancy; blood transfusion; and prior exposure to the specific donor's HLA. In certain embodiments, the recipient's medical history includes prior exposure to HLA. In certain embodiments the recipient has donor specific antibodies prior to desensitization. In certain embodiments, the prior exposure to HLA includes one or more of prior solid organ or tissue allograft, pregnancy, blood transfusion, or prior exposure to the specific donor's HLA.

In certain embodiments, the desensitization therapy comprises plasmapheresis treatment. Often the desensitization therapy comprises intravenous immune globulin treatment (IVIg).

The duration of the desensitization therapy may be at least about 1 day, about 1 week, or about 2 weeks.

In certain embodiments, the therapeutically effective dose of the anti-C5 antibody, or an antigen binding fragment thereof, is administered in a phased dosing schedule that comprises about a 1200 mg dose administered about 1 hour prior to kidney allograft reperfusion. Often dose of the anti-C5 antibody, or an antigen binding fragment thereof, comprises about a 900 mg dose administered at about day 1, about day 7, about day 14, about day 21, and about day 28 post transplantation. The effective dose of the anti-C5 antibody, or an antigen binding fragment thereof, may comprise about a 1200 mg dose administered at about week 5; about week 7, and about week 9 post transplantation. The effective dose of the anti-C5 antibody, or an antigen binding fragment thereof, may comprise about a 1200 mg dose administered about 1 hour prior to kidney allograft reperfusion; about a 900 mg dose is administered at about day 1, about day 7, about day 14, about day 21, and about day 28; and a 1200 mg dose is administered at about week 5; about week 7, and about week 9 post transplantation. In some embodiments, the therapeutically effective dose includes a 1200 mg dose on the day of the transplant, and 900 mg of eculizumab on the following post-transplantation days: 1, 7, 14 (±2 days) and 21 (±2 days). The therapeutically effective dose further usually also includes administering 1200 mg of eculizumab on the following post-transplantation weeks: week 5 (±2 days), week 7 (±2 days) and week 9 (±2 days).

In certain embodiments, on the day of the transplant the anti-C5 antibody (e.g., eculizumab) may be administered prior to reperfusion of the kidney allograft. Often the anti-C5 antibody (e.g., eculizumab) is administered from about 30 minutes to about 3 hours prior to reperfusion of the kidney allograft. In certain embodiments, the he anti-C5 antibody (e.g., eculizumab) is administered about 1 hour prior to reperfusion of the kidney allograft.

In certain embodiments, the day 1 dose of the anti-C5 antibody (e.g., eculizumab) is administered from about 18 to about 30 hours after reperfusion of the kidney allograft. In certain embodiments, the day 1 dose is administered about 24 hours after reperfusion of the kidney allograft.

In certain embodiments, the recipient's plasma levels of anti-C5 antibody, or an antigen binding fragment thereof, is maintained at about 50 to about 100 μg/mL for about the first week post transplantation. In some embodiments, the recipient's plasma levels of anti-C5 antibody, or an antigen binding fragment thereof is maintained at about 50 to about 100 μg/mL for about the first 9 weeks post transplantation. Methods for measuring or determining plasma levels of an antibody are known in the art.

In certain embodiments, the recipient has a historical positive complement-dependent cytotoxicity cross-match. The recipient may have a B cell flow cytometric cross-match from about 300 to about 500 mean channel shift. Sometimes the recipient has a T cell flow cytometric cross-match from about 300 to about 500 mean channel shift.

The recipient may have a donor specific antibody identified by a single antigen bead assay with a single mean fluorescence intensity greater than about 3000. The recipient may have a single mean fluorescence intensity from about 3000 to about 6000. Sometimes, the recipient has a single mean fluorescence intensity from about 3000 to about 12000.

A diagnosis of AMR may be based on the presence of circulating anti-donor specific antibodies, and morphologic evidence of acute tissue injury. The evidence of acute tissue injury may be based on a biopsy. A diagnosis of AMR may be based on the recipient exhibiting histological findings consistent with Banff Class II or III AMR on transplant biopsy.

The method of the disclosure may further comprise administering at least one immunosuppressive drug to the recipient. Exemplary immunosuppressive drug include tacrolimus, mycophenolate mofetil, and prednisone.

In further embodiments, the method may include a step of administering plasmapheresis to the recipient. The method may also include a step of administering immunoglobulin to the recipient. In some embodiments the method may also include a step of administering both plasmapheresis and immunoglobulin to the recipient.

The symptoms of AMR in the recipient may include acute graft dysfunction, (elevation of creatinine above post-transplant nadir) and often includes two out of three, of the following: the presence of circulating donor specific antibodies; histological findings consistent with Banff Class II or III AMR on transplant biopsy and, peritubular capillary c4d positivity on transplant biopsy.

The recipient may be a human adult between 18 and 75 years of age.

### Outcomes

In some embodiments, in the methods disclosed herein, the recipient experiences a clinically meaningful low level of circulating anti-donor specific antibodies during about the first 9 weeks post transplantation compared to the absence of therapy with the antibody or antigen binding fragment thereof.

In some embodiments, in the methods disclosed herein, the recipient experiences a clinically meaningful low level of circulating anti-donor specific antibodies during about the first 12 months post transplantation, compared to the absence of therapy with the antibody or antigen binding fragment thereof.

In some embodiments, in the methods disclosed herein, the recipient experiences clinically meaningful low level of morphologic evidence of acute tissue injury during about the first 9 weeks post transplantation, compared to the absence of therapy with the antibody or antigen binding fragment thereof.

In some embodiments, in the methods disclosed herein, the recipient experiences clinically meaningful low level of morphologic evidence of acute tissue injury during about the first 12 months post transplantation, compared to the absence of therapy with the antibody or antigen binding fragment thereof.

In some embodiments, in the methods disclosed herein, the recipient experiences a clinically meaningful increase in graft survival at about week 9 post transplantation, compared to the absence of therapy with the antibody or antigen binding fragment thereof.

In some embodiments, in the methods disclosed herein, the recipient experiences a clinically meaningful increase in graft survival at about month 12 post transplantation, compared to the absence of therapy with the antibody or antigen binding fragment thereof.

In some embodiments, in the methods disclosed herein, the recipient experiences increased survival at about 9-weeks post transplantation compared to the absence of therapy with the antibody or antigen binding fragment thereof.

In some embodiments, in the methods disclosed herein, the recipient experiences increased survival at about 12-months post transplantation compared to the absence of therapy with the antibody or antigen binding fragment thereof.

In some embodiments, in the methods disclosed herein, the recipient experiences increased survival at about 36-months post transplantation compared to the absence of therapy with the antibody or antigen binding fragment thereof.

In some embodiments, in the methods disclosed herein, the recipient experiences clinically meaningful low histological evidence of antibody mediated rejection during about the first 9 weeks post transplantation, compared to the absence of therapy with the antibody or antigen binding fragment thereof.

In some embodiments, in the methods disclosed herein, the recipient experiences clinically meaningful low histological evidence of antibody mediated rejection during about the first 12 months post transplantation, compared to the absence of therapy with the antibody or antigen binding fragment thereof.

In some embodiments, in the methods disclosed herein, the recipient experiences a clinically meaningful low pathological changes, including chronic AMR, on biopsies during about the first 9 weeks post transplantation, compared to the absence of therapy with the antibody or antigen binding fragment thereof.

In some embodiments, in the methods disclosed herein, the recipient experiences a clinically meaningful low pathological changes, including chronic AMR, on biopsies during about the first 12 months post transplantation compared to the absence of therapy with the antibody or antigen binding fragment thereof.

In some embodiments, in the methods disclosed herein, the recipient has reduced need for plasmapheresis treatments during about the first 9 weeks post transplantation compared to the absence of therapy with the antibody or antigen binding fragment thereof.

In some embodiments, in the methods disclosed herein, the recipient has reduced need for plasmapheresis treatments during about the first 12 months post transplantation compared to the absence of therapy with the antibody or antigen binding fragment thereof.

In some embodiments, in the methods disclosed herein, the recipient experiences clinically meaningful reduced delayed graft function post transplantation compared to the absence of therapy with the antibody or antigen binding fragment thereof.

In some embodiments, in the methods disclosed herein, the recipient experiences clinically meaningful reduction in need for dialysis during about the first 9 weeks post transplantation, compared to the absence of therapy with the antibody or antigen binding fragment thereof.

In some embodiments, in the methods disclosed herein, the recipient experiences a clinically meaningful reduction in need of dialysis during about the first 12 months post transplantation, compared to the absence of therapy with the antibody or antigen binding fragment thereof.

In some embodiments, in the methods disclosed herein, the recipient experiences stable renal function during about the first 9 weeks post transplantation compared to the absence of therapy with the antibody or antigen binding fragment thereof.

In some embodiments, in the methods disclosed herein, the recipient experiences stable renal function during about the first 12 months post transplantation compared to the absence of therapy with the antibody or antigen binding fragment thereof.

In certain embodiments, the method of the disclosure results in the kidney allograft surviving for at least six months. In certain embodiments, the kidney allograft may survive for at least one year. In certain embodiments, the kidney allograft survives for at least three years. The kidney allograft may survive for at least five years. The method may result in the kidney allograft surviving for the remaining life-time of the recipient.

### Compositions and Formulations

This disclosure provides methods that comprise administering an anti-C5 antibody, or an antigen binding fragment thereof, to a patient (a kidney transplant recipient), wherein the anti-C5 antibody, or binding fragment is contained within a pharmaceutical composition. These pharmaceutical compositions are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical compositions, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Routes of administration may be any suitable route and may include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition disclosed herein can be delivered subcutaneously or intravenously with a standard needle and syringe. In addition, with respect to subcutaneous delivery, a pen delivery device readily has applications in delivering a pharmaceutical composition of the invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the invention. Examples include, but are not limited to, AUTOPEN™ (Owen Mumford, Inc., Woodstock, UK), DISETRONIC™ pen (Disetronic Medical Systems, Bergdorf, Switzerland), HUMALOG MIX75/25™ pen, HUIIVIALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present invention include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

For direct administration to the sinuses, the pharmaceutical compositions disclosed herein may be administered using, e.g., a microcatheter (e.g., an endoscope and microcatheter), an aerosolizer, a powder dispenser, a nebulizer or an inhaler. The methods include administration of an anti-C5 antibody, or an antigen binding fragment thereof, to a subject in need thereof, in an aerosolized formulation. For example, aerosolized anti-C5 antibody, or an antigen binding fragment thereof may be administered to treat asthma in a patient. Aerosolized antibodies can be prepared as described in, for example, U.S. Pat. No. 8,178,098, incorporated herein in its entirety.

In certain situations, the pharmaceutical composition can be delivered in a controlled release system. In one embodiment, a pump may be used (see Langer, 1990, Science 249:1527-1533; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

The pharmaceutical compositions for oral or parenteral use described above may be prepared into dosage forms in a unit dose suited to fit a dose of the active ingredients. Such dosage forms in a unit dose include, for example, tablets, pills, capsules, injections (ampoules), suppositories, etc.

The dose of antibody administered to a patient according to the methods disclosed herein may vary depending upon the age and the size of the patient, symptoms, conditions, route of administration, and the like. The preferred dose maybe typically calculated according to body weight or body surface area. Depending on the severity of the condition, the frequency and the duration of the treatment can be adjusted. Effective dosages and schedules for administering pharmaceutical compositions comprising anti-C5 antibody, or an antigen binding fragment thereof may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

The anti-C5 antibody, or an antigen binding fragment thereof, can be administered as a fixed dose, or in a milligram per kilogram (mg/kg) dose. While in no way intended to be limiting, exemplary dosage ranges include, e.g., 1-100 μg/kg, 0.5-50 μg/kg, 0.1-100 μg/kg, 0.5-25 μg/kg, 1-20 μg/kg, and 1-10 μg/kg, 1-100 mg/kg, 0.5-50 mg/kg, 0.1-100 mg/kg, 0.5-25 mg/kg, 1-20 mg/kg, and 1-10 mg/kg. Exemplary dosages of the anti-C5 antibody, or antigen-binding fragment thereof, include, without limitation, 0.1 μg/kg, 0.5 μg/kg, 1.0 μg/kg, 2.0 μg/kg, 4 μg/kg, and 8 μg/kg, 0.1 mg/kg, 0.5 mg/kg, 1.0 mg/kg, 2.0 mg/kg, 4 mg/kg, and 8 mg/kg.

The amount of anti-C5 antibody, or an antigen binding fragment thereof, administered to a subject according to the methods disclosed herein is, generally, a therapeutically effective amount. As used herein, the phrase “therapeutically effective amount” means an amount of anti-C5 antibody, or an antigen binding fragment thereof, that reduces the likelihood that the recipient will develop antibody mediated rejection, compared to the absence of therapy.

A therapeutically effective amount of the anti-C5 antibody, or an antigen binding fragment thereof, can be from about 100 mg to about 2500 mg, e.g., about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, and about 2000 mg. In certain embodiments, 600 mg, 900, 1200 or 1500 of the anti-C5 antibody, or an antigen binding fragment thereof antibody is administered.

In certain embodiments, the anti-C5 antibody or an antigen-binding fragment thereof is administered through intravenous infusion or subcutaneously.

In certain embodiments, the methods disclosed herein further comprise administering to the recipient one or more immunosuppressive drug selected from the group consisting of tacrolimus, mycophenolate mofetil, and prednisone.

### Kits

This disclosure also provides kits comprising the anti-C5 antibody, or antigen-binding fragment thereof, or compositions thereof (or unit dosages forms and/or articles of manufacture) and may further comprise instruction(s) on the methods of use disclosed herein. The kits described herein may further include other materials desirable from a commercial and user standpoint, including other buffers, diluents, filters, needles, syringes, and package inserts with instructions for performing any methods described herein.

### Examples

### Example 1: A Randomized, Open-Label, Multicenter Trial to Determine Safety and Efficacy of Eculizumab in the Prevention of AMR in Living Donor Kidney Transplant Recipients Requiring Desensitization Therapy

A randomized, multicenter, open-label, phase II, two-arm parallel study was conducted to demonstrate the safety and efficacy of eculizumab at reducing the risk of AMR in sensitized recipients of living donor kidney transplants requiring desensitization therapy prior to transplantation.

The primary objective of this study was to demonstrate the safety and efficacy of eculizumab to prevent AMR in sensitized recipients of living donor kidney transplants requiring desensitization therapy prior to transplantation. Secondary objectives include characterization of the overall safety and tolerability of eculizumab compared with placebo and characterization of the efficacy of eculizumab compared with placebo by additional efficacy measures, including graft function, subject and graft survival and biopsy proven acute rejection.

Subjects that were desensitized and cleared for transplantation were randomized to either the Treatment Arm or the SOC Control Arm. The Eculizumab Treatment Arm patients received eculizumab (study drug) for 9 weeks post transplantation. The SOC Control Arm patients received prophylactic therapy for AMR according to the SOC choice at each participating investigative site.

The following is a list of abbreviations that may be used in this example.

TABLE 4
List of Abbreviations

| Abbreviation | |
|---|---|
| or Specialist | |
| Term | Explanation |
| ABO | A, B and 0 Blood Glycoproteins (Blood Type) |
| ACR | Acute Cellular Rejection |
| AE | Adverse Event |
| aHUS | Atypical Hemolytic Uremic Syndrome |
| ALT | Alanine aminotransferase (SGPT) |
| AMR | Antibody-Mediated Rejection |
| AP | Alkaline Phosphatase |
| AST | Aspartate aminotransferase (SGOT) |
| BFXM | B-Cell Cytometric Flow Crossmatch |
| BE | Bioequivalence |
| BID | Twice Daily |
| BK | BK Virus |
| BUN | Blood Urea Nitrogen |
| ° C. | Degrees Celsius |
| CBC | Complete Blood Count |
| CI | Confidence Interval |
| Cₘₐₓ | Peak Concentration |
| CDC | Complement-Dependent Cytotoxicity |
| CDRs | Complementarity Determining Regions |
| CHR | Chronic Humoral Rejection |
| CMR | Cell Mediated Rejection |
| CMV | Cytomegalovirus |
| CMVIg | Anti-Cytomegalovirus Hyperimmune Globulin |
| CRF | Case Report Form |
| CRO | Clinical Research Organization |
| CsA | Cyclosporine |
| CyP | Cyclophosphamide |
| DD | Deceased Donor |
| DGF | Delayed Graft Function |
| DMC | Data Monitoring Committee |
| DSA | Donor Specific Antibody |
| ECG | Electrocardiogram |
| eGFR | Estimated Glomerular Filtration Rate |
| ELISA | Enzyme Linked Immunosorbent Assay |
| EMA | European Medicines Agency |
| ESRD | End-Stage Renal Disease |
| ° F. | Degrees Fahrenheit |
| FCXM | Flow Cytometric Crossmatch |
| FDA | Food and Drug Administration |
| FFP | Fresh Frozen Plasma |
| FSH | Follicle-Stimulating Hormone |
| GCP | Good Clinical Practice |
| GGT | Gamma-Glutamyltransferase |
| β-HCG | Beta-Human Chorionic Gonadotrophic Hormone |
| HBV | Hepatitis B Virus |
| HCT | Hematocrit |
| HCV | Hepatitis C Virus |
| HD | High Dose |
| HEENT | Head, Ears, Eyes, Nose, Throat |
| Hgb | Hemoglobin |
| HIV | Human Immunodefinacy Virus |
| HLA | Human Leukocyte Antigen |
| ICF | Informed Consent Form |
| ICH | International Conference on Harmonization |
| IEC | Independent Ethics Committee |
| IgG | Immunoglobulin G |
| INR | International Normalized Ratio |
| IRB | Institutional Review Board |
| IV | Intravenous |
| IVIg | Intravenous Immune Globulin |
| kDa | Kilodalton |
| LD | Live Donor |
| LDH | Lactate Dehydrogenase |
| mAb | Monoclonal Antibody |
| MAC | Membrane Attack Complex |
| MCH | Mean Corpuscular Hemoglobin |
| MCHC | Mean Corpuscular Hemoglobin Concentration |
| MCP-1 | Monocyte Chemotactic Protein 1 |
| MCS | Mean Channel Shift |
| MCV | Mean Corpuscular Volume |
| MDRD7 | Modification of Diet in Renal Disease 7 |
| MedDRA | Medical Dictionary for Regulatory Activities |
| MFI | Mean Fluorescence Intensity |
| MHC | Major Histocompatibility Complex |
| MMF | Mycophenolate Mofetil |
| PCP | Pneumocystis carininPneumocystisjiroveci Pneumonia |
| PCR | Polymerase Chain Reaction |
| PD | Pharmacodynamics |
| PE | Plasma Exchange |
| PK | Pharmacokinetics |
| Plts | Platelets |
| PP | Plasmapheresis |
| POD | Post-operative Day |
| PNH | Paroxysmal Nocturnal Hemoglobinuria |
| PRA | Percent Reactive Antibody |
| PT | Prothrombin Time |
| PTT/aPTT | Partial Thromboplastin Time/activated Partial |
| | Thromboplastin Time |
| RBC | Red Blood Cells |
| SAE | Serious Adverse Event |
| SAB | Single-bead Antigen |
| SCr | Serum Creatinine |
| SOC | Standard of Care |
| TAC | Tacrolimus |
| TEAE | Treatment Emergent Adverse Event |
| TFXM | T-Cell Cytometric Flow Crossmatch |
| XM | Crossmatch |
| WBC | White Blood Cells |

### Overall Study Design

After appropriately screened patients were enrolled in the study, it was anticipated that patients would undergo about two or more weeks of desensitization therapy prior to transplantation. The actual length of desensitization for an individual patient was based on the clinical judgment of their transplant team. Patients desensitized and cleared for transplantation by the Principal Investigator were randomized to either the Treatment Arm or the SOC Control Arm of the study. All patients received standard immunosuppression, prophylactic medications and post transplantation care. The diagnosis of AMR for the determination of the primary end point was based on “for cause” kidney biopsies. In addition, protocol biopsies were performed on all patients at predetermined time points. All patients were screened for standard laboratory values, DSA titers, TFXM, BFXM, complement-dependent cytotoxicity (CDC), estimated glomerular filtration rate (eGFR), and other clinical and laboratory parameters for evaluation of primary and secondary endpoints as well as safety. The primary analysis of the data occurred after all patients had reached Month 12 post transplantation. However, both study arms had additional follow up at Months 18, 24, and 36 post transplantation to assess patient and graft survival, kidney disease, and disease status.

### Study Period (Years)

This study was estimated to require approximately 5 years for completion. The following were the expected major timelines for this study: (a) estimated date first patient enrolled: 4th Q2011; (b) estimated date last patient, first visit: 4th Q2013; (c) estimated date last patient completed: 4th Q2014; and (d) estimated date of last patient completing 3 year follow up data collection: 4^{th} Q2016.

It was estimated that approximately 45 kidney Transplant Centers in North America, EU, and Australia would be required to fully enroll this study.

### Screening/Enrollment:

Patients considered candidates to receive a living donor kidney transplant by the investigative sites' selection criteria and sensitized to their living donor as defined below were considered for enrollment in this study. Candidates for enrollment signed an informed consent form (ICF) and underwent the baseline screening at the investigative site's Local Laboratory with duplicate specimens being sent to the Central Laboratory for confirmation. The Central Laboratory specimen values were utilized for verification that the candidates meet enrollment criteria for study entry and subsequent desensitization.

### Infectious Disease Screening:

A pre-transplant infectious disease evaluation was performed as part of the screening assessment for all patients. An evaluation to determine the epidemiological risks, past medical history, and/or ongoing evidence of infection in patients was performed in preparation for transplantation. Preferably this evaluation was done by an infectious diseases (ID) physician.

The evaluation included risk factors for Aspergillus. Pre-Screening tests for those patients with identified epidemiological risk factors as determined by past medical history included chest CT and microbiological tests including induced sputum culture and serum galactomannan testing as deemed necessary per findings and/or the recommendation of an ID specialist.

N. meningitidis Vaccination

If all inclusion criteria and none of the exclusion criteria were met, patients were vaccinated against N. meningitidis (if not already vaccinated within the time period of active coverage specified by the vaccine manufacturer). All patients were vaccinated as soon after signature on the informed consent as possible and then re-vaccinated 30 days later. If the patient was successfully desensitized prior to the 30 day booster vaccination, the trial center proceeded to transplantation provided that the patient received his/her initial vaccination at least 14 days prior to the first dose of eculizumab. A booster dose was administered 30 days after the initial dose. If a patient has previously been vaccinated, within the time period of active coverage by the vaccine manufacturer prior to enrollment in this trial, only a booster dose was required after the patient signed consent. If the 30 day booster vaccine fell within the first week post-transplant, the booster was postponed until the second week post-transplant. Tetravalent conjugated vaccines for N. meningitidis was required for this trial. Individual clinical trial centers had the option to provide patients with prophylactic antibiotics during eculizumab treatment. A sub-study to evaluate the immune response to meningococcal vaccination was performed on 20 patients at selected centers.

### Screening for Sensitization

Enrollment in this study was restricted to patients who were candidates for living donor kidney transplantation and who were sensitized to their living donors in accordance with the three baseline parameters delineated below. Patients meeting these criteria continued in the study and proceeded to desensitization.

Although duplicate samples were collected for local laboratory assessment of DSA, CDC, and flow crossmatching, the Central Laboratory specimens were used to select patients for study eligibility and desensitization.

Sensitizing events (determined by Investigator documented history of prior exposure to human leukocyte antigens [HLA]). For example (not an all-inclusive list): (a) prior solid organ or tissue allograft; (b) pregnancy; (c) blood transfusion; and (d) prior exposure to donor's HLA.

If the clinical history was consistent with donor specific antibody (DSA) exposure then: (a) DSA was identified by single antigen bead (SAB) assay (Luminex LabScreen assay), as described by the manufacturer's package insert and performed at the study's Central Laboratory.

If DSA by SAB was present then: patients had a positive complement-dependent cytotoxicity (CDC) cross match (current or historic) or a positive B-cell flow cross match (BFXM) and/or T-cell flow cross match (TFXM) according to the Central Laboratory.

### Randomization

All patients who were adequately desensitized and determined to be safe to transplant by the PI and the local center's standard practice be randomized 1:1 to either the treatment or SOC control arm. Eculizumab Treatment Arm: Patients received one dose of eculizumab approximately one hour prior to reperfusion of the allograft and were treated with eculizumab for 9 weeks post transplantation. SOC Control Arm: Patients were treated post transplantation with the Transplant Center's SOC for prophylaxis for AMR. In addition, the randomization was stratified by the pre-transplant desensitization protocol that was used: (a) plasmapheresis (PP) and intravenous immunoglobulin (IVIg); (b) PP alone; (c) IVIg alone.

Desensitization Protocol

Patients who met all of the inclusion and none of the exclusion criteria and who had been confirmed eligible from results obtained from the Central Laboratory were desensitized to their living donor using their Transplant Center's SOC protocol: plasmapheresis (PP) and/or intravenous immunoglobulin (IVIg).

Rituximab was prohibited in all patients as part of the pre-transplantation desensitization therapy due to a potential drug-drug interaction.

Each Transplant Center selected as a desensitization protocol as their SOC. The desensitization protocol selected must be used uniformly for all patients at that center throughout the study. Completion of the desensitization process was based on the clinical practice of the Transplant Center.

Following desensitization all patients were determined to be safe for transplant by the standard of practice at the transplanting Center. This evaluation included at least one of the following: CDC cross match, cell based flow cross match, or SAB (single antigen bead) testing.

Laboratory samples at the Local Laboratory were obtained following desensitization and prior to transplantation to assess desensitization and clearance for transplantation. Duplicate samples were obtained and sent to the Central Laboratory. The Local Laboratory specimens were used by the Principal Investigator to judge suitability for transplantation. The Luminex-based SAB assay results from both the central and local labs were collected and included in the study database.

### Primary Endpoint

The primary composite endpoint is the Week 9 post transplantation treatment failure rate defined as the occurrence of a) biopsy-proven AMR; b) graft loss; c) patient death; or d) loss to follow-up.

The diagnosis of AMR was based on kidney allograft dysfunction and biopsy performed “for cause.” The histological diagnosis was based on Banff 2007 criteria see Table 19) for AMR as determined by the Central Pathology Laboratory. For this study only level II and level III AMR were accepted as defined below: (a) Presence of circulating anti-donor specific antibodies, morphologic evidence of acute tissue injury, such as (Type/Grade); (b) Banff 2007 level II—Capillary and/or glomerular inflammation (ptc/g >0) and/or thromboses; (c) Banff 2007 level III—Arterial—v3; (d) Secondary Endpoints.

Secondary endpoints for this study included the following: Cumulative incidence of AMR that occurs between Week 9 and Month 12 post transplantation (AMR of any grade that meets Banff 2007 criteria); Treatment failure rate defined as the occurrence of 1) biopsy-proven AMR, 2) graft loss, 3) patient death, or 4) loss to follow-up at Month 12 post transplantation; Graft and patient survival at Months 6 and 12 post transplantation; histological evidence of AMR on protocol biopsies without other clinical findings at Day 14, and Months 3 and 12 post transplantation; overall pathological changes including chronic AMR, on protocol biopsies at Day 14, and Months 3 and 12 post transplantation; cumulative number of PP treatments at 12 months post transplantation; cumulative incidence of patients requiring splenectomy at 12 months post transplantation; incidence of delayed graft function (DGF) post transplantation (defined as the requirement for dialysis within the first post transplantation week for reasons other than post-operative hyperkalemia, acute pulmonary edema, or fluid overload due to comorbid conditions); cumulative incidence and duration of dialysis between 7 days and 12 months post transplantation; stable renal function between Week 4 and Month 12 post transplantation as measured by: estimated Glomerular Filtration Rate (calculated) by Modification of Diet in Renal Disease 7 (MDRD7); serum creatinine defined as the value on at least 3 consecutive measurements taken at least 2 days apart while not on PP or dialysis that vary ≤20%.

### Number of Patients:

An estimated 90 patients were enrolled and randomized to the study with approximately 45 patients enrolled per arm. This was based on a two-arm binomial proportion study for the primary efficacy endpoint variable. To attain 90 transplanted patients, it was estimated that the study would identify 130 patients eligible to enroll to randomize 90 patients. See Statistics and Data Analysis section for additional details.

### Treatment Assignment and Duration of Treatment

Patients underwent desensitization therapy according to the practice of the Local Transplant Center prior to transplantation. Following desensitization all patients were determined to be safe to transplant by the standard of practice at the Transplant Center. This evaluation included at least one of the following: CDC cross match, cell based flow cross match, or SAB (single antigen bead) testing.

Patients were followed for primary and secondary endpoints to Month 12 post transplantation, and for DSA, kidney function and patient and graft survival up to Month 36 post transplantation.

Patients diagnosed with clinically significant biopsy-proven AMR during the first 9 weeks of treatment were considered treatment failures. Investigators were allowed to treat the AMR with eculizumab in addition to other agents. See dosing instructions for both eculizumab and SOC study arms for AMR occurring during the Week 9 treatment period. In addition, for AMR that is diagnosed after 9 weeks, Investigators may also use eculizumab as part of the AMR treatment regimen. See dosing instructions for both eculizumab and SOC study arms for AMR occurring after the Week 9 treatment period.

### Eculizumab Treatment Arm

All doses of eculizumab were administered IV as a continuous infusion over 35-45 minutes. Treatment started during the transplantation procedure and continued as follows: Eculizumab 1200 mg (4 vials) administered in the operating room approximately 1 hour prior to kidney allograft reperfusion (day 0); Eculizumab 900 mg (3 vials) on the following post transplantation days: day 1; day 7; day 14 (+/−2 days); day 21 (+/−2 days); and day 28 (+/−2 days). Eculizumab 1200 mg (4 vials) given on the following post transplantation weeks: week 5 (+/−2 days); week 7 (+/−2 days); week 9 (+/−2 days). PP and/or IVIg were used to treat diagnosed AMR that occurred at any time, in the eculizumab treatment arm. In this setting the study drug should continue to be administered per the guidelines below.

### SOC Control Arm

Patients who were randomized to the SOC control arm received prophylactic therapy for AMR post transplantation according to the Local Transplant Center protocol and included in any combination: PP and/or IVIg. Rituximab was prohibited in all patients except for the treatment of rejection at the discretion of the PI. SOC treatments was used uniformly for all patients at a given center on a center-specific basis. If patients in the SOC arm were diagnosed with AMR, they were treated with first line therapy (PP and/or IVIg). They were treated with eculizumab in conjunction with other therapies.

### Dose Adjustment Criteria

Eculizumab was administered intravenously as a fixed dose depending upon the time relative to the transplant as listed above.

### Safety Criteria for Adjustment or Stopping Doses

If an adverse reaction occurred during the administration of eculizumab, the infusion was slowed or stopped at the discretion of the Principal Investigator. If the infusion was slowed, the total infusion time did not exceed two hours. The adverse reaction was recorded on the AE page of the CRF.

### Infusion Reactions

As with all protein products, administration of eculizumab may have resulted in infusion reactions, including anaphylaxis or other hypersensitivity reactions. Patients were monitored of for at least one hour following completion of the infusion for signs or symptoms of an infusion reaction. Eculizumab administration was interrupted in all patients experiencing severe infusion reactions and appropriate medical therapy administered. The infusion reaction was recorded on the AE page of the CRF.

### Criteria for Study Termination

The Data Monitoring Committee (DMC) was in charge of monitoring the risk-benefit ratio for the patients and could make the following recommendation to the Sponsor: (a) continued enrollment and dosing of the Eculizumab Treatment Arm; (b) enroll at a reduced dose in Eculizumab Treatment Arm; (c) Increase monitoring of patients in Eculizumab Treatment Arm; (d) Recommend stopping dosing in Eculizumab Treatment Arm.

### Study Procedures

### General Information

Transplant recipients were cared for according to the investigative site's SOC protocols employed for post transplantation follow-up. The Principal Investigator at each site was directly responsible for supervising the care of these recipients during the length of the study.

### Laboratory Information

Sites utilized Local Laboratories for the following tests: Hematology Panel; Chemistry Panel; Urinalysis' Spot urine for urine protein/creatinine ratio; Tacrolimus Trough; Activated Partial Prothromplastin Time (aPTT), PT (Prothrombin Time) and International Normalized Ratio (INR); Fibrinogen/Fibrinogen Split Products (should be collected on CRFs as part of routine post plasmapheresis therapy labs); eGFR (MDRD 7); Serum Pregnancy Test for Women of Childbearing Potential (See Section 0 for exemptions); BFXM and/or TFXM for routine management (Local [if available] and Central Laboratories [mandatory]); CDC (Local [if available] and Central Laboratories); The DSA by Luminex LabScreen (Local and Central Laboratories).

### Central Laboratory Information

A Central Laboratory was responsible for blinded analysis of BFXM, TFXM, CDC, and DSA by Luminex LabScreen taken at predetermined times (See Schedule of Assessments in Tables 5-8). PK/PD samples were forwarded by the sites to ACM Laboratories for accessioning and storage until the end of the study.

### Central Pathology Information

All protocol and “for cause” kidney biopsies were processed and analyzed by the site's Local Pathology Laboratory. Processed slides and two paraffin embedded unstained slides for immunohistochemical studies were forwarded to the Central Pathology imaging center for blinded review by a panel of independent pathologists. Additional details about processing and review of the slides for the Central Pathology Laboratory were found in the Pathology Laboratory Manual.

### Clinical Assessments

Clinical assessments were conducted routinely during the post-operative period according to the transplant site protocol and also at various time points throughout the study. These assessments included an assessment of the patient's health status, renal function and new diagnoses.

### Female Patients of Child-Bearing Potential

Female candidates who were of child-bearing potential had a negative pregnancy test (serum beta-hCG) and practice a medically approved contraceptive regimen during the post transplantation period for at least 5 months following discontinuation of eculizumab. Female patients were exempt from contraception requirement if they were post-menopausal for at least 1 year before dosing or were surgically sterile (i.e., no uterus or no ovaries). Females who had their fallopian tubes banded, tied, or cut were not considered surgically sterile without FSH level confirmation. Note: although females of child bearing age with end stage renal disease (ESRD) can be amenorrheic, they must still meet all standards for contraception or surgical sterility prior to transplantation.

### Timing of Visits and Missed Visits

The schedule for clinical assessments during the Pre-Transplant, Immediate Post transplant, Extended Post transplant, and Long Term Outcome Phases are located in Tables 8, 9, 10, and 11. For practical logistical reasons the assigned visit windows were designed to allow more flexibility after the initial 9 weeks of the study. In all cases, if a study visit was missed or outside of the assigned visit window it was expected that a protocol deviation was documented on the appropriate forms.

### Standard of Care Patients AMR Prophylaxis Study Visits

Patients randomized to the SOC control arm received prophylactic therapy for AMR post transplantation according to the investigative site's protocol. This therapy included PP and IVIg in any combination but avoided prohibited medications. The patients were required to complete all study visits as per the Schedule of Assessments but received their prophylaxis treatments at the time points determined by the investigative site's SOC protocol. The time points for the SOC prophylaxis therapy may or may not correspond with the Schedule of Assessments visit schedule. Therefore all the information on the SOC prophylaxis therapy was captured on the designated CRF's to include the type of therapy, the time and date of administration, and the duration of the therapy. All other information for the study visit for SOC patients was completed on the days identified in the Schedule of Assessments.

### Pre-Treatment Phase

The following procedures were performed during the Screening period:

### Pre-Transplant Week −8 to −3

Informed consent; Demographics; Medical history; Complete physical exam including vital signs, height and weight; Determination of eligibility based on inclusion/exclusion criteria; Infectious disease screening 12-lead electrocardiogram (ECG); Hematology panel; Chemistry panel; Urinalysis; aPTT, PT, and INR; Serum pregnancy test for women of childbearing potential for exemptions); Vaccination against N. meningitides; BFXM and/or TFXM (samples to Local [if possible] and Central Laboratories [mandatory]); Collect donor blood for local and Central Laboratory testing; CDC (samples to Local [if available] and Central Laboratories); DSA by Luminex LabScreen (samples to Local and Central Laboratories); Record concomitant medications; Assessment of AEs.

Entry criteria for the study was determined by Central Laboratory data for DSA, CDC, BFXM, and TFXM at Screening.

### Pre-Transplant Week −3 to Week −1 (Days −21, −14, −7 Prior to Transplant)

The length of time over which a patient was desensitized was dependent upon when DSA levels that would allow transplantation were achieved, in the opinion of the Principal Investigator. If the patient achieved these DSA levels by Week −2, the Week −1 visit was omitted providing all labs below were drawn. Patients then generally proceeded to the Week 0 (Day −1) visit. The Local Laboratory specimen data was used for patient management purposes.

All patients were vaccinated as soon after signature on the informed consent as possible and then re-vaccinated 30 days later according to current medical guidelines for vaccination use. If the patient was successfully desensitized prior to the 30 day booster vaccination, the trial center ensured that the patient has received the initial vaccination at least 14 days prior to the first dose of eculizumab and then the booster dose was administered at the 30 days after the initial dose. If a patient had previously been vaccinated prior to enrollment in this trial, only a booster dose was required after the patient signed consent. If the 30 day booster vaccine fell within the first week post-transplant, the booster was postponed until the second week post-transplant. Tetravalent conjugated vaccines for N. meningitidis were required for this trial. Individual clinical trial centers had the option to provide patients with prophylactic antibiotics at any time during eculizumab treatment.

Pre-Transplant PP and IVIg began at least 48 hours after the patient received the vaccination against N. meningitidis. Dates, dosage, time duration and laboratory values were collected in the CRFs including fibrinogen/fibrinogen split products.

Abbreviated physical exam included vital signs and weight; determination that the patient met the criteria for ‘sensitized’; DSA by Luminex LabScreen (samples to Local and Central Laboratories); recorded concomitant medications; and assessment of AEs. All the information on administration of the desensitization therapy was captured on designated case report forms (CRFs) to include the type of therapy, the time and date of administration, and the duration of the therapy.

The following visits and procedures were applicable only for patients who meet the criteria for transplantation after desensitization per the SOC at each respective Transplant Center.

### Pre-Transplant Week 0 (Day −1 Prior to Transplant)

Once the patient had been desensitized and cleared for transplantation by the Principal Investigator, the following procedures was completed 1 day prior to transplantation: abbreviated physical exam including vital signs and weight; assessment of inclusion/exclusion criteria conformity; BFXM and/or TFXM (samples to Local [if available] and Central Laboratories [mandatory]); CDC (samples to Local [if available] and Central Laboratories); DSA by Luminex LabScreen (samples to Local and Central Laboratories); randomization to study arm (Eculizumab Treatment Arm or SOC Control Arm); record concomitant medications; assessment of AEs; instruct the patient on the signs and symptoms of N. meningitides; provide Patient Identification and Safety Card to the patient explaining that the patient is participating in a clinical trial with a description of the Investigational Product, emergency contact information, and the signs and symptoms of N. meningitidis.

### Immediate Post Transplant Phase

The Local Laboratory specimen data for BFXM, TFXM (if possible), and DSA will used for patient management.

During the study, patients carried a detailed card describing the “alert” symptoms for Neisseria meningitides at all times. The triggers for seeking immediate medical attention were any of the following symptoms: headache with nausea or vomiting; headache with fever; headache with a stiff neck or back; fever of 103° F. (39.4° C.) or higher; fever and a rash; confusion; severe myalgia with flu-like symptoms and, sensitivity to light.

### Transplant Day 0/Week 0

For all patients, the following were completed on the day of the transplant: kidney transplant procedure; complete physical exam including vital signs and weight; 12-lead ECG; hematology panel; complete chemistry panel; urinalysis; aPTT, PT, and INR; BFXM and/or TFXM (samples to Local [if available] and Central Laboratories [mandatory]); DSA by Luminex LabScreen (samples to Local and Central Laboratories); assess renal function/need for dialysis; kidney allograft biopsy (post-reperfusion; the slides that were read locally are to be sent to Central Pathology for digitization and independent pathology read); record concomitant medications; record immunosuppressive medications; and assessment of AEs.

For Treatment Arm Patients only, the following are completed: administer eculizumab, 1200 mg (4 vials), approximately one hour prior to reperfusion of kidney allograft; and baseline and peak PK and PD collection (baseline samples should be taken 5-90 minutes prior to study drug infusion; peak samples are to be taken 60 minutes after the completion of the study drug infusion).

For SOC Control Arm Patients only, the following are performed: prophylactic therapy for AMR post transplantation according to the investigative sites SOC protocol. It may have include PP and IVIg for site specific durations and may not have correlated precisely with study visit days; Recorded fibrinogen/fibrinogen split products as part of routine post PP labs in CRFs.

### Post-Transplant Day 1/Week 0

For all patients, the following were completed one day post transplantation: abbreviated physical exam including vital signs and weight; clinical assessment including evaluation for rejection (assessment to be from local laboratory results and performed by Principal Investigator or appropriately appointed designee at the Transplant Center); hematology panel; complete chemistry panel; aPTT, PT, and INR; tacrolimus trough; BFXM and/or TFXM (samples to Local [if available] and Central Laboratories [mandatory]); DSA by Luminex LabScreen (samples to Local and Central Laboratories); assess renal function/need for dialysis; record concomitant medications; record immunosuppressive medications; assessment of AEs.

For Treatment Arm Patients only, the following were completed: administer eculizumab, 900 mg (3 vials); trough and peak PK and PD collection (trough samples were taken 5-90 minutes prior to study drug infusion; peak samples were to be taken 60 minutes after the completion of the study drug infusion).

For SOC Control Arm Patients only, the following: prophylactic therapy for AMR post transplantation according to the investigative sites SOC protocol. It included PP and IVIg for site specific durations and may not have correlated precisely with study visit days. Record fibrinogen/fibrinogen split products as part of routine post PP labs in CRFs.

### Post-Transplant Days 2-6/Week 0

For all patients the following were completed: abbreviated physical exam including vital signs and weight; clinical assessment including evaluation for rejection; hematology panel; abbreviated chemistry panel; aPTT, PT, and INR (Day 2 and 3 only); tacrolimus trough; assess renal function/need for dialysis; record concomitant medications; record immunosuppressive medications; and assessment of AEs.

For SOC Control Arm Patients only, the following were performed: prophylactic therapy for AMR post transplantation according to the investigative sites SOC protocol. It may have included PP and IVIg for site specific durations and may not have correlated precisely with study visit days; and recorded fibrinogen/fibrinogen split products as part of routine post PP labs in CRFs.

### Post-Transplant Day 7/Week 1

For all patients the following were completed: abbreviated physical exam including vital signs and weight; clinical assessment including evaluation for rejection; hematology panel; complete chemistry panel; urinalysis; spot urine for urine protein/creatinine ratio; tacrolimus trough; BFXM and/or TFXM (samples to Local [if available] and Central Laboratories [mandatory]); DSA by Luminex LabScreen (samples to Local and Central Laboratories); assess renal function/needed for dialysis; recorded concomitant medications; recorded immunosuppressive medications; assessment of AEs.

For Treatment Arm Patients only, the following were completed: administered eculizumab, 900 mg (3 vials). Trough and peak PK and PD collection (trough samples were taken 5-90 minutes prior to study drug infusion; peak samples were to be taken 60 minutes after the completion of the study drug infusion).

For SOC Control Arm Patients only, the following: prophylactic therapy for AMR post transplantation according to the investigative sites SOC protocol. It may have included PP and IVIg for site specific durations and may not have correlated precisely with study visit days; recorded fibrinogen/fibrinogen split products as part of routine post PP labs in CRFs.

### Extended Post Transplant Phase

All patients continued to be seen for study visits at regular intervals Post transplant Day 14 through Month 12 (primary efficacy analysis). The Local Laboratory specimen data for BFXM, TFXM (if possible), and DSA were used for patient management.

### Post-Transplant Day 14/Week 2

For all patients, the following were completed: abbreviated physical exam including vital signs and weight; clinical assessment including evaluation for rejection; hematology panel; abbreviated chemistry panel; aPTT, PT, and INR; tacrolimus trough; BFXM and/or TFXM (samples to Local [if available] and Central Laboratories [mandatory]); DSA by Luminex LabScreen (samples to Local and Central Laboratories); assess renal function/need for dialysis; kidney allograft biopsy (send the locally read slides to Central Pathology); record concomitant medications; record immunosuppressive medications; and assessment of AEs.

For Treatment Arm Patients only, the following were completed (+/−2 days): Administer eculizumab, 900 mg (3 vials); trough and peak PK and PD collection (trough samples should be taken 5-90 minutes prior to study drug infusion; peak samples were taken 60 minutes after the completion of the study drug infusion).

For SOC Control Arm Patients only, the following were performed: prophylactic therapy for AMR post transplantation according to the investigative sites SOC protocol. It may have included PP and IVIg for site specific durations and may not have correlated precisely with study visit days; recorded fibrinogen/fibrinogen split products as part of routine post PP labs in CRFs.

### Post-Transplant Day 21/Week 3

For all patients, the following were completed: abbreviated physical exam including vital signs and weight; clinical assessment including evaluation for rejection; hematology panel; abbreviated chemistry panel; aPTT, PT, and INR; tacrolimus trough; BFXM and/or TFXM (samples to Local [if available] and Central Laboratories [mandatory]); DSA by Luminex LabScreen (samples to Local and Central Laboratories); Assess renal function/need for dialysis; record concomitant medications; record immunosuppressive medications; and assessment of AEs.

For Treatment Arm Patients only, the following were completed (+/−2 days): Administer eculizumab, 900 mg (3 vials). No PK/PD assessments required for this dose.

For SOC Control Arm Patients only, the following were performed: prophylactic therapy for AMR post transplantation according to the investigative sites SOC protocol. It may have included PP and IVIg for site specific durations and may not have correlated precisely with study visit days; recorded fibrinogen/fibrinogen split products as part of routine post PP labs in CRFs.

### Post-Transplant Day 28/Week 4

For all patients, the following were completed: abbreviated physical exam including vital signs and weight; clinical assessment including evaluation for rejection; hematology panel; abbreviated chemistry panel; urinalysis; spot urine for urine protein/creatinine ratio; aPTT, PT, and INR; tacrolimus trough; BFXM and/or TFXM (samples to Local [if available] and Central Laboratories [mandatory]); DSA by Luminex LabScreen (samples to Local and Central Laboratories); assess renal function/need for dialysis; eGFR (MDRD 7); record concomitant medications; record immunosuppressive medications; and assessment of AEs.

For Treatment Arm Patients only, the following were completed (+/−2 days): administer eculizumab, 900 mg (3 vials); trough and peak PK and PD collection (trough samples should be taken 5-90 minutes prior to study drug infusion; peak samples were taken 60 minutes after the completion of the study drug infusion).

For SOC Control Arm Patients only, the following were performed: prophylactic therapy for AMR post transplantation according to the investigative sites SOC protocol. It may have included PP and IVIg for site specific durations and may not have correlated precisely with study visit days; and recorded fibrinogen/fibrinogen split products as part of routine post PP labs in CRFs.

### Post-Transplant Days 35 and 49/Weeks 5 and 7

For all patients, the following were completed: abbreviated physical exam including vital signs and weight; clinical assessment including evaluation for rejection; SCr and BUN; tacrolimus trough; assess renal function/need for dialysis; record concomitant medications; record immunosuppressive medications; and assessment of AEs.

For Treatment Arm Patients only, the following were completed (+/−2 days): Administer eculizumab, 1200 mg (4 vials); Trough and peak PK and PD collection (trough samples should be taken 5-90 minutes prior to study drug infusion; peak samples were taken 60 minutes after the completion of the study drug infusion).

For SOC Control Arm Patients only, the following: prophylactic therapy for AMR post transplantation according to the investigative sites SOC protocol. It may have included PP and IVIg for site specific durations and may not have correlated precisely with study visit days; and recorded fibrinogen/fibrinogen split products as part of routine post PP labs in CRFs.

### Post-Transplant Day 56/Week 8

For all patients, the following were completed: abbreviated physical exam including vital signs and weight (optional per standard of care for this investigative site); clinical assessment including evaluation for rejection; hematology panel; abbreviated chemistry panel; tacrolimus trough; assess renal function/need for dialysis; eGFR (MDRD 7); other required information (will be obtained from the patient via telephone by the Principal Investigator or the appropriate designee at the Transplant Center); record concomitant medications; record immunosuppressive medications; and assessment of AEs.

For SOC Control Arm Patients only, the following were performed: prophylactic therapy for AMR post transplantation according to the investigative sites SOC protocol. It may have included PP and IVIg for site specific durations and may not have correlated precisely with study visit days; and recorded fibrinogen/fibrinogen split products as part of routine post PP labs in CRFs.

### Post-Transplant Day 63/Week 9

For all patients, the following were completed: abbreviated physical exam including vital signs and weight; clinical assessment including evaluation for rejection; hematology panel; abbreviated chemistry panel; urinalysis; spot urine for urine protein/creatinine ratio; tacrolimus trough; BFXM and/or TFXM (samples to Local [if available] and Central Laboratories [mandatory]); DSA by Luminex LabScreen (samples to Local and Central Laboratories); assess renal function/need for dialysis; eGFR (MDRD 7); record concomitant medications; record immunosuppressive medications; and assessment of AEs.

For Treatment Arm Patients only, the following were completed (+/−2 days): administer eculizumab, 1200 mg (4 vials); and trough and peak PK and PD collection (trough samples were taken 5-90 minutes prior to study drug infusion; peak samples were taken 60 minutes after the completion of the study drug infusion).

For SOC Control Arm Patients only, the following were performed: prophylactic therapy for AMR post transplantation according to the investigative sites SOC protocol. It may have included PP and IVIg for site specific durations and may not have correlated precisely with study visit days. Record fibrinogen/fibrinogen split products as part of routine post PP labs in CRFs.

### Post-Transplant Week 12/Month 3

For all patients, the following were completed: complete physical exam including vital signs and weight; clinical assessment including evaluation for rejection; hematology panel; abbreviated chemistry panel; Urinalysis; Spot urine for urine protein/creatinine ratio; Tacrolimus trough; BFXM and/or TFXM (samples to Local [if available] and Central Laboratories [mandatory]); DSA by Luminex LabScreen (samples to Local and Central Laboratories); assess renal function/need for dialysis; eGFR (MDRD 7); kidney allograft biopsy (the slides that were read locally are to be sent to Central Pathology for digitization and independent pathology read); record concomitant medications; record immunosuppressive medications; and assessment of AEs.

For SOC Control Arm Patients only, the following were performed: prophylactic therapy for AMR post transplantation according to the investigative sites SOC protocol. It may include PP and IVIg for site specific durations and may not have correlated precisely with study visit days; and recorded fibrinogen/fibrinogen split products as part of routine post PP labs in CRFs.

### Post-Transplant Weeks 17 & 21/Months 4 & 5

For all patients the following were completed: abbreviated physical exam including vital signs and weight (optional per standard of care for this investigative site); clinical assessment including evaluation for rejection (assessment to be from local laboratory results and performed by Principal Investigator or appropriately appointed designee at the Transplant Center); SCr and BUN; tacrolimus trough; assess renal function/need for dialysis; other required information (will be obtained from the patient via telephone by the Principal Investigator or the appropriate designee at the Transplant Center); record concomitant medications; record immunosuppressive medications; and assessment of AEs.

For SOC Control Arm Patients only, the following were performed: prophylactic therapy for AMR post transplantation according to the investigative sites SOC protocol. It may have included PP and IVIg for site specific durations and may not have correlated precisely with study visit days; and recorded fibrinogen/fibrinogen split products as part of routine post PP labs in CRFs

### Post-Transplant Week 26/Month 6

For all patients, the following were completed: complete physical exam including vital signs and weight; clinical assessment including evaluation for rejection; hematology panel; abbreviated chemistry panel; urinalysis; spot urine for urine protein/creatinine ratio; aPTT, PT, and INR; tacrolimus trough; BFXM and/or TFXM (samples to Local [if available] and Central Laboratories [mandatory]); DSA by Luminex LabScreen (samples to Local and Central Laboratories); assess renal function/need for dialysis; eGFR (MDRD 7); record concomitant medications; record immunosuppressive medications; and assessment of AEs;

For SOC Control Arm Patients only, the following were performed: prophylactic therapy for AMR post transplantation according to the investigative sites SOC protocol. It may have included PP and IVIg for site specific durations and may not have correlated precisely with study visit days; and record fibrinogen/fibrinogen split products as part of routine post PP labs in CRFs

### Post-Transplant Weeks 30 & 34/Months 7 & 8

For all patients, the following were completed: abbreviated physical exam including vital signs and weight (optional per standard of care for this investigative site); clinical assessment including evaluation for rejection (assessment to be from local laboratory results and performed by Principal Investigator or appropriately appointed designee at the Transplant Center); SCr and BUN; tacrolimus trough; and assess renal function/need for dialysis; other required information (will be obtained from the patient via telephone by the Principal Investigator or the appropriate designee at the Transplant Center); record concomitant medications; record immunosuppressive medications; and assessment of AEs.

For SOC Control Arm Patients only, the following were performed: prophylactic therapy for AMR post transplantation according to the investigative sites SOC protocol. It may have included PP and IVIg for site specific durations and may not have correlated precisely with study visit days. Record fibrinogen/fibrinogen split products as part of routine post PP labs in CRFs

### Post-Transplant Week 38/Month 9

For all patients, the following were completed: abbreviated physical exam including vital signs and weight (optional per standard of care for this investigative site); clinical assessment including evaluation for rejection (assessment to be from local laboratory results and performed by Principal Investigator or appropriately appointed designee at the Transplant Center); hematology panel; abbreviated Chemistry panel; tacrolimus trough; assess renal function/need for dialysis; eGFR (MDRD 7). Other required information (will be obtained from the patient via telephone by the Principal Investigator or the appropriate designee at the Transplant Center); record concomitant medications; record immunosuppressive medications; and assessment of AEs.

For SOC Control Arm Patients only, the following were performed: prophylactic therapy for AMR post transplantation according to the investigative sites SOC protocol. It may have included PP and IVIg for site specific durations and may not have correlated precisely with study visit days. Record fibrinogen/fibrinogen split products as part of routine post PP labs in CRFs.

### Post-Transplant Weeks 44 & 48/Months 10 & 11

For all patients, the following were completed: abbreviated physical exam including vital signs and weight (optional per standard of care for this investigative site); clinical assessment including evaluation for rejection (assessment to be from local laboratory results and performed by Principal Investigator or appropriately appointed designee at the Transplant Center); SCr and BUN; tacrolimus trough; assess renal function/need for dialysis; other required information (will be obtained from the patient via telephone by the Principal Investigator or the appropriate designee at the Transplant Center); record concomitant medications; record immunosuppressive medications; and assessment of AEs.

For SOC Control Arm Patients only, the following were performed: prophylactic therapy for AMR post transplantation according to the investigative sites SOC protocol. It may include PP and IVIg for site specific durations and may not have correlated precisely with study visit days; and record fibrinogen/fibrinogen split products as part of routine post PP labs in CRFs

### Post-Transplant Week 52/Month 12—Study Primary Analysis Time Point

For all patients, the following were completed: complete physical exam including vital signs and weight; clinical assessment including evaluation for rejection; hematology panel; abbreviated chemistry panel; urinalysis; spot urine for urine protein/creatinine ratio; aPTT, PT, and INR; tacrolimus trough; BFXM and/or TFXM (sampled to Local [if available] and Central Laboratories [mandatory]); DSA by Luminex LabScreen (samples to Local and Central Laboratories); assess renal function/need for dialysis; eGFR (MDRD 7); kidney allograft biopsy (the slides that were read locally are to be sent to Central Pathology for digitization and independent pathology read); record concomitant medications; record immunosuppressive medications; and assessment of AEs.

For SOC Control Arm Patients only, the following were performed: prophylactic therapy for AMR post transplantation according to the investigative sites SOC protocol. It may include PP and IVIg for site specific durations and may not correlate precisely with study visit days; and, record fibrinogen/fibrinogen split products as part of routine post PP labs in CRFs.

### Long Term Outcomes Phase

Additional study visits occurred at Months 18, 24, and 36 for long term follow up data. This data was not used for purposes of the primary efficacy analysis.

### Post-Transplant Months 18 and 24

For all patients, the following were completed: assessment of rejection episodes in interim from last visit, patient survival, graft loss and kidney disease and disease status; chemistry panel; tacrolimus trough; other immunosuppressant levels; assessment of AEs.

### Post-Transplant Month 36

For all patients, the following were completed: assessment of rejection episodes in interim from last visit, patient survival, graft survival; kidney disease, and disease status; chemistry panel; tacrolimus trough; other immunosuppressant levels; BFXM and/or TFXM (samples to Central Laboratory only); DSA by Luminex LabScreen (sample to Central Laboratory only); kidney allograft biopsy (locally read slides to Central Pathology Laboratory); and assessment of AEs

### Definition of the End of the Trial:

The end of the trial was defined as the Last Visit Last Patient.

### Treatment of Persistent DSA Levels

DSA was analyzed both by central and local laboratory in both arms of the study during treatment, at the end of the treatment period (Week 9), and at Months 3, 6, 12 and 36. Central Laboratory results from week 9 only will be provided to the local centers. If the recipient maintains a positive DSA and/or a positive BFXM and/or TFXM as measured by the Central Laboratory and/or Local Laboratory testing (week 9 result) then PP and/or IVIg may be used to lower the DSA as follows:

SOC arm: PP and/or IVIg will be administered per the clinical judgment of the Principal Investigator. Supplementary medications (eculizumab, rituximab, bortezomib) are not allowed to treat persistent DSA.

Eculizumab arm: PP and/or IVIg were administered per the clinical judgment of the Principal Investigator. Supplementary eculizumab as a booster following PP may have been administered during weeks 9-10 only. In this case eculizumab (600 mg) was administered within 1 hour following each treatment of PP. Other medications such as rituximab and bortezomib were not allowed to treat persistent DSA.

Note that Eculizumab supplementation was not allowed for treatment of persistent DSA that extends beyond the 10^{th} postoperative week. Prior to Week 9, patients in the eculizumab arm were treated according to treatment assignment guidelines in the Sections entitled Investigational Product, Dosage and Mode of Administration and Reference Therapy, Dosage and Mode of Administration.

Treatment with Fresh Frozen Plasma

If a patient received FFP not associated with PP, then patients receiving eculizumab would also receive a supplemental dose of eculizumab (600 mg) 1 hour prior to FFP administration.

### Screen Failure

Patients who did not meet the study criteria during the Screening phase and/or could not proceed with the desensitization protocol were considered screen failures. These patients were discontinued from the study without follow-up.

### Medical-Social Failure or Desensitization Failure

### Failure to Receive a Transplant:

Screened patients who developed other medical or social reasons that prevented them from subsequently undergoing kidney transplantation, were discontinued from the study. A discontinuation CRF form that documented the reason for the Screening failure was completed.

### Desensitization Failures:

The protocol did require a specific reduction in DSA prior to entry into a treatment arm of the study. The decision to proceed with entering a patient into a treatment arm of the study after the completion of desensitization was at the discretion of the Principal Investigator. If the Investigator determined that a patient should not continue in the study after completion of desensitization, a discontinuation CRF form that documented the reason for the decision to withdraw the patient was completed.

Other patients who were considered a desensitization failure were replaced as required for the study to meet its objectives.

### Premature Discontinuations and Withdrawals

### Early Termination Withdrawals or Discontinuations:

Reasons for early discontinuation or withdrawal were documented completely in the appropriate CRF.

If a patient discontinued the eculizumab study drug at any time during the study, the patient had additional study visits to ensure safety follow-up every 2 weeks for 2 months (maximum of 4 visits) following the final dose. These visits may coincided with routine follow-up visits for maintenance of their kidney transplant per the Transplant Center. The last visit included all assessments listed for the Month 12 visit in the Schedule of Assessments.

### Schedules of Assessments

TABLE 5
Schedule of Assessment - Pre-Transplant Phase

| | Pre-Transplant Study Visit | | | | | |
|---|---|---|---|---|---|---|
| | | Pre- | Pre- | Pre- | Pre- | Pre- |
| | Informed¹ | Transplant² | Transplant² | Transplant² | Transplant² | Transplant |
| | Consent | Day −56¹ | Day −21 | Day −14 | Day −7 | Day −1 |
| | Study Week | | | | | |
| | | Week −8 | | | | |
| | | to −3 | Week −3 | Week −2 | Week −1 | Week 0 |
| | Visit Window | | | | | |
| | 3 months | 56 to 21 | | | | |
| | prior to | days prior | | +/−1 | +/−1 | +/−1 |
| Procedure | desensitization | to transplant | 0 | day | day | day |
| Informed Consent | X | | | | | |
| (Recipient & Donor) | | | | | | |
| Demographics | X | | | | | |
| Medical History | X | | | | | |
| Physical Exam including | X | | | | | |
| Vital Signs, Height and | | | | | | |
| Weight | | | | | | |
| Infectious Disease | X | | | | | |
| Assessment | | | | | | |
| Abbreviated Physical | | | X | X | X⁴ | X |
| Exam including Vital | | | | | | |
| Signs and Weight³ | | | | | | |
| Assessment of | X | | | | | X |
| Inclusion/Exclusion | | | | | | |
| Conformity | | | | | | |
| 12-lead ECG | X | | | | | |
| Vaccination against | X | | | | | X |
| N. meningitidis⁵ | | | | | | |
| Pre-Transplant PP, IVIg⁶ | | X | X | X | X⁴ | |
| and post-therapy | | | | | | |
| fibrinogen/fibrinogen split | | | | | | |
| products | | | | | | |
| Chemistry Panel including | X | | | | | |
| SCr and BUN | | | | | | |
| Hematology Panel | X | | | | | |
| including WBC diff., Plts, | | | | | | |
| Hgb | | | | | | |
| Urinalysis | X | | | | | |
| aPTT, PT, and INR | X | | | | | |
| Serum Pregnancy Test for | X | | | | | |
| Women of Childbearing | | | | | | |
| Potential | | | | | | |
| BFXM and TFXM | X⁷ | | | | | X⁷ |
| CDC | X⁷ | | | | | X⁷ |
| DSA by Luminex LabScreen | X⁷ | X⁷ | X⁷ | X⁷ | X⁴ | X⁷ |
| Randomization/Stratification | | | | | | X |
| Provide Patient | | | | | | X |
| Identification and Safety | | | | | | |
| Card | | | | | | |
| Concomitant Medications | X | X | X | X | X⁴ | X |
| Adverse Event Assessment | | X | X | X | X⁴ | X |

¹If a patient is initially screened greater than 3 months prior to desensitization, the patient must undergo the assessments indicated for Pre-Transplant Day 56 within the specified window timeframe. If a patient is screened within 56 days of desensitization, assessment for the Pre-Transplant Day 56 are not indicated.
²It is anticipated that patients will undergo 2 or more weeks of desensitization prior to transplant. The actual length of desensitization for an individual patient will be based on the clinical judgment of their transplant team.
³Abbreviated physical examination consists of a body system relevant examination based upon Investigator judgment and patient symptoms.
⁴If the DSA level that clears a patient for transplantation is attained (per Investigator's discretion) at Week −2 and the transplant can be performed, the Week −1 visit may be omitted. Complete the labs and assessments for the Day −1 Pre-transplant visit and randomization can occur.
⁵All patients must be vaccinated as soon after signing the informed consent as possible and then re-vaccinated 30 days later according to current medical guidelines for vaccination use. If tire patient is successfully desensitized prior to the 30 day booster vaccination, the trial center must ensure that the patient has received the initial vaccination at least 14 days prior to the first dose of eculizumab and then the booster dose is to be administered at the 30 day period. If a patient has previously been vaccinated prior to enrollment in this trial, only a booster dose is required after the patient signs consent.
⁶Information on desensitization therapy, including type of therapy, time & date of administration, duration of therapy, and fibrinogen/fibrinogen split products will be captured on designated CRFs.
⁷Samples for BFXM, TFXM, CDC and DSA levels must be sent to the Central Laboratory for the Screening and Day −1. Samples should also be sent to the Local Laboratory when possible for BFXM, TFXM, CDC and DSA levels according to the site's capabilities. The Central Laboratory specimen data will be used for confirmation of study entry criteria at Screening. At all other interim time points during Screening selected by the Investigative Site for patient management, the Local Laboratory will be used for processing of specimens. These interim samples do not need to be sent to the Central Laboratory. See Study Manual for sample processing information.

TABLE 6
Schedule of Assessment - Immediate Post Transplant Phase

| | Transplant Study Visit | | | | | | | |
|---|---|---|---|---|---|---|---|---|
| | Transplant, | | | | | | | |
| | Day 0 | Day 1 | Day 2 | Day 3 | Day 4 | Day 5 | Day 6 | Day 7 |
| | Study Week | | | | | | | |
| | Week 0 | Week 0 | | | | | | Week 1 |
| | Visit Window | | | | | | | |
| Procedure | 0 | 0 | 0 | 0 | 0 | 0 | 0 | 0 |
| Kidney | X | | | | | | | |
| Transplantation | | | | | | | | |
| Physical Exam | X | | | | | | | |
| including Vital Signs | | | | | | | | |
| and Weight | | | | | | | | |
| Abbreviated Physical | | X | X | X | X | X | X | X |
| Exam including Vital | | | | | | | | |
| Signs and Weight¹ | | | | | | | | |
| 12-lead ECG | X | | | | | | | |
| Clinical Assessment | | X | X | X | X | X | X | X |
| including Evaluation | | | | | | | | |
| for Rejection | | | | | | | | |
| Post-Transplant, | X³ | X⁴ | | | | | | X⁴ |
| Treatment Arm Only: | | | | | | | | |
| Administer | | | | | | | | |
| Eculizumab² | | | | | | | | |
| Post-Transplant, | X⁵ | X⁵ | X⁵ | X⁵ | X⁵ | X⁵ | X⁵ | X⁵ |
| Standard of Care | | | | | | | | |
| Arm Only: PP and | | | | | | | | |
| IVIg⁵ (capture | | | | | | | | |
| fibrinogen/ | | | | | | | | |
| fibrinogen split | | | | | | | | |
| products post- | | | | | | | | |
| therapy) | | | | | | | | |
| Hematology Panel | X | X | X | X | X | X | X | X |
| including WBC diff., | | | | | | | | |
| Plts, Hgb | | | | | | | | |
| Abbreviated | | | X | X | X | X | X | |
| Chemistry Panel⁶ | | | | | | | | |
| Complete Chemistry | X | X | | | | | | X |
| Panel⁷ | | | | | | | | |
| PK and PD⁸ | B/P | T/P | | | | | | T/P |
| Urinalysis | X | | | | | | | X |
| Spot Urine for Urine | | | | | | | | X |
| Protein/Creatinine | | | | | | | | |
| Ratio | | | | | | | | |
| aPTT, PT, and INR | X | X | X | X | | | | |
| Tacrolimus trough | | X | X | X | X | X | X | X |
| BFXM and TFXM⁹ | X | X | | | | | | X |
| DSA by Luminex | X | X | | | | | | X |
| LabScreen⁹ | | | | | | | | |
| Assess renal function/ | X | X | X | X | X | X | X | X |
| need for dialysis | | | | | | | | |
| Kidney Allograft | X | | | | | | | |
| Biopsy (post | | | | | | | | |
| reperfusion)¹⁰ | | | | | | | | |
| Concomitant | X | X | X | X | X | X | X | X |
| Medications | | | | | | | | |
| Immunosuppressive | X | X | X | X | X | X | X | X |
| Medications | | | | | | | | |
| Adverse Event | X | X | X | X | X | X | X | X |
| Assessment | | | | | | | | |

¹Abbreviated physical examination consists of a body system relevant examination based upon Investigator judgment and patient symptoms.
²No PP or IVIg may be administered in the eculizumab Treatment Arm during first 9 weeks unless biopsy-proven AMR.
³Administer eculizumab 1200 mg (4 vials) IV over 35-45 minutes one horn prior to re-perfusion of kidney.
⁴Administer eculizumab 900 mg (3 vials) IV on Days 1 and 7 post-transplantation over 35-45 minutes.
⁵Standard of care for investigative site; patients receive prophylaxis therapy for AMR post-transplant post transplantation according to the Local Transplant Center protocol and may include PP and IVIg in any combination for site specific durations. Treatments may not precisely correlate with study visits, but dates, times, dosages and lab values (including fibrinogen/fibrinogen split products) must be captured on appropriate CRF's. All other visit assessments should be completed on the designated dates.
⁶Abbreviated chemistry panel consists of sodium, potassium, chloride. BUN, creatinine, and glucose.
⁷Completed chemistry panel consists of all laboratory parameters included in the abbreviated chemistry panel with the addition of carbon dioxide, total cholesterol. AST, potassium, albumin, total protein, ALT, alkaline phosphatase, calcium, magnesium, phosphorus, uric acid, LDH, GGT, and total and direct bilirubin.
⁸Only required for Active Treatment Arm of Study. B = Baseline sample; T = Trough sample; P = Peak sample. Baseline and trough samples for PK/PD are to be taken 5-90 minutes before the study drug infusion. Peak samples for PK/PD testing are to be taken 60 minutes after the completion of the study drug infusion. See Study Manual for sample processing information.
⁹BFXM, TFXM and DSA levels are to be drawn on Days 0, 1, and 7 and run at the Local Laboratory if available. Duplicate samples are to be sent to the Central Laboratory. At all other interim time points selected by the investigative site for patient management, the Local Laboratory will be used for processing of specimens. These interim samples do not need to be sent to the Central Laboratory. See Study Manual for sample processing information. Local Laboratory specimen data will be used for all patient management. See Study Manual for sample processing information.
¹⁰The slides that were read locally are to be sent to Central Pathology for digitization and independent pathology read.

TABLE 7
Schedule of Assessment - Extended Post Transplant Phase

| | Transplant Study Visit | | | | | | | |
|---|---|---|---|---|---|---|---|---|
| | Day 14 | Day 21 | Day 28 | Days 35 & 49 | Day 56 | Day 63 | Mo. 3 | Mo. 4 & 5 |
| | Study Week | | | | | | | |
| | Week 2 | Week 3 | Week 4 | Week 5 & 7 | Week 8 | Week 9 | Week 12 | Week 17 & 21 |
| | Visit Window | | | | | | | |
| Procedure | +/−2 days | +/−2 days | +/−2 days | +/−2 days | +/−2 days | +/−2 days | +/−7 days | +/−7 days |
| Physical Exam Including | | | | | | | X | |
| Vital Signs and Weight | | | | | | | | |
| Abbreviated Physical Exam | X | X | X | X | X² | X | | X² |
| Including Vital Signs and | | | | | | | | |
| Weight¹ | | | | | | | | |
| Clinical Assessment | X | X | X | X | X | X | X | X |
| including Evaluation for | | | | | | | | |
| Rejection | | | | | | | | |
| Post-Transplant, Treatment | X⁴ | X⁴ | X⁴ | X⁵ | | X⁵ | | |
| Arm Only: Administer | | | | | | | | |
| Eculizumab³ | | | | | | | | |
| Post-Transplant, Standard | X⁶ | X⁶ | X⁶ | X⁶ | X⁶ | X⁶ | X⁶ | X⁶ |
| of Care Arm Only: PP and | | | | | | | | |
| IVIg⁶ (capture fibrinogen/ | | | | | | | | |
| fibrinogen split products | | | | | | | | |
| post-therapy) | | | | | | | | |
| Hematology Panel including | X | X | X | | X | X | X | |
| WBC diff, Plts, Hgb | | | | | | | | |
| Abbreviated Chemistry Panel | X | X | X | X⁷ | X | X | X | X⁷ |
| including SCr and BUN | | | | | | | | |
| PK and PD | T/P⁸ | | T/P⁸ | T/P⁸ | | T/P⁸ | | |
| Urinalysis | | | X | | | X | X | |
| Spot Urine for Urine | | | X | | | X | X | |
| Protein/Creatinine Ratio | | | | | | | | |
| aPTT, PT, and INR | X | X | X | | | | | |
| Tacrolimus trough | X | X | X | X | X | X | X | X |
| BFXM and TFXM⁹ | X | X | X | | | X | X | |
| DSA by Luminex LabScreen⁹ | X | X | X | | | X | X | |
| Assess Renal Function/need | X | X | X | X | X | X | X | X |
| for dialysis | | | | | | | | |
| eGFR (MDRD 7) | | | X | | X | X | X | |
| Kidney Allograft Biopsy | X | | | | | | X | |
| Concomitant Medications | X | X | X | X | X¹⁰ | X | X | X¹⁰ |
| Immunosuppressive | X | X | X | X | X¹⁰ | X | X | X¹⁰ |
| Medications | | | | | | | | |
| Adverse Event Assessment | X | X | X | X | X¹⁰ | X | X | X¹⁰ |

| | Transplant Study Visit | | | | |
|---|---|---|---|---|---|
| | Mo. 6 | Mo. 7 & 8 | Mo. 9 | Mo. 10 & 11 | Mo. 12 |
| | Study Week | | | | |
| | Week 26 | Week 30 & 34 | Week 38 | Week 44 & 48 | Week 52 |
| | Visit Window | | | | |
| Procedure | +/−7 days | +/−7 days | +/−7 days | +/−7 days | +/−7 days |
| Physical Exam Including | X | | | | X |
| Vital Signs and Weight | | | | | |
| Abbreviated Physical Exam | | X² | X² | X² | |
| Including Vital Signs and | | | | | |
| Weight¹ | | | | | |
| Clinical Assessment | X | X | X | X | X |
| including Evaluation for | | | | | |
| Rejection | | | | | |
| Post-Transplant, Treatment | | | | | |
| Arm Only: Administer | | | | | |
| Eculizumab³ | | | | | |
| Post-Transplant, Standard | X⁶ | X⁶ | X⁶ | X⁶ | X⁶ |
| of Care Arm Only: PP and | | | | | |
| IVIg⁶ (capture fibrinogen/ | | | | | |
| fibrinogen split products | | | | | |
| post-therapy) | | | | | |
| Hematology Panel including | X | | X | | X |
| WBC diff, Plts, Hgb | | | | | |
| Abbreviated Chemistry Panel | X | X⁷ | X | X⁷ | X |
| including SCr and BUN | | | | | |
| PK and PD | | | | | |
| Urinalysis | X | | | | X |
| Spot Urine for Urine | X | | | | X |
| Protein/Creatinine Ratio | | | | | |
| aPTT, PT, and INR | X | | | | X |
| Tacrolimus trough | X | X | X | X | X |
| BFXM and TFXM⁹ | X | | | | X |
| DSA by Luminex LabScreen⁹ | X | | | | X |
| Assess Renal Function/need | X | X | X | X | X |
| for dialysis | | | | | |
| eGFR (MDRD 7) | X | | X | | X |
| Kidney Allograft Biopsy | | | | | X |
| Concomitant Medications | X | X¹⁰ | X¹⁰ | X¹⁰ | X |
| Immunosuppressive | X | X¹⁰ | X¹⁰ | X¹⁰ | X |
| Medications | | | | | |
| Adverse Event Assessment | X | X¹⁰ | X¹⁰ | X¹⁰ | X |

¹Abbreviated physical examination consists of a body system relevant examination based upon Investigator judgment and patient symptoms.
²Abbreviated physical exam at Day 56 (Week 8), and Months 4, 5, 7, 8, 9, 10, and 11 are optional per standard of care for investigative site.
³No prophylactic PP or IVIg may be administered in the Treatment Arm during first 9 weeks unless biopsy-proven AMR.
⁴Administer eculizumab 900 mg (3 vials) IV on Days 14, 21, 28 over 35-45 minutes.
⁵Administer eculizumab 1200 mg (4 vials) IV at Weeks 5, 7, 9 over 35-45 minutes.
⁶Standard of care for investigative site; patients receive prophylaxis therapy for AMR post transplantation according to the Local Transplant Center protocol and may include PP and IVIg in any combination for site-specific durations. Treatments may not precisely correlate with study visits, but dates, times, dosages, and lab values (including fibrinogen/fibrinogen split product post-therapy) must be captured on appropriate CRF's. All other visit assessments should be completed on the designated dates.
⁷SCr and BUN only.
⁸T = Trough sample; P = Peak sample. Trough samples for PK/PD are to be taken 5-90 minutes before the study drug infusion. Peak samples for PK/PD testing are to be taken 60 minutes after the completion of the study drug infusion. See Study Manual for sample processing information.
⁹BFXM, TFXM, and DSA levels are monitored on Days 14, 21, 28, Week 9 and Months 3, 6, and 12 at the Local Laboratory. Duplicate samples are to be sent to Central Laboratory. At all other interim time points selected by the investigative site for patient management, the Local Laboratory will be used for processing of specimens. These interim samples do not need to be sent to the Central Laboratory. See Study Manual for sample processing information. Local Laboratory specimen data will be used for all patient management. See Study Manual for sample processing information.
¹⁰Will be obtained from the patient via telephone by the Principal Investigator or the appropriate designee at the Transplant Center

TABLE 8
Schedule of Assessment - Long Term Outcome Data Collection

| | Post-Transplant Study Visit | | |
|---|---|---|---|
| | Month 18 | Month 24 | Month 36 |
| | Study Week | | |
| | Week 78 | Week 104 | Week 156 |
| | Visit Window | | |
| Procedure | +/−14 days | +/−14 days | +/−14 days |
| Medical Assessments for | X | X | X |
| Interim Rejection Episodes, | | | |
| Graft Loss, Patient Survival, | | | |
| Kidney Disease, and Disease | | | |
| Status¹ | | | |
| Chemistry Panel including | X | X | X |
| SCr and BUN | | | |
| Tacrolimus Trough | X | X | X |
| Other Immunosuppressant | X | X | X |
| Levels | | | |
| BFXM and TFXM² | | | X |
| DSA by Luminex LabScreen² | | | X |
| Kidney Allograft Biopsy³ | | | X |
| Adverse Event Assessment | X | X | X |

¹Interim rejection episodes will be recorded from previous visit through subsequent visit.
²BFXM, TFXM, and DSA specimens to be sent to the Central Laboratory only. See Study Manual for sample processing information.
³Slides that are read locally will be sent to Central Pathology Laboratory.

### Selection and Withdrawal of Patients:

All patients adhered to the following inclusion/exclusion criteria.

Patient Inclusion Criteria:

Male or female patients ≥18 years old; Patients with Stage IV or Stage V chronic kidney disease who would receive a kidney transplant from a living donor to whom they are sensitized and require desensitization prior to transplantation; History of prior exposure to HLA. For example (not an all-inclusive list): a) Prior solid organ or tissue allograft; b. Pregnancy; c. Blood transfusion; d. Prior exposure to specific donor's HLA; The presence of DSA by the SAB assay (Luminex LabScreen assay), as described by the manufacturer's package insert and performed at the study's Central Laboratory; Positive CDC cross match (current or historic) OR have a negative CDC cross match and a positive BFXM and/or TFXM according to the Central Laboratory; Able to understand the informed consent form and willing to comply with study procedures; Female patients of child-bearing potential must have a negative pregnancy test (serum beta-hCG) and must be practicing an effective, reliable, and medically approved contraceptive regimen while on eculizumab treatment and for up to 5 months following discontinuation of treatment.

Patient Exclusion Criteria:

Has received treatment with eculizumab at any time prior to enrolling in this study; ABO incompatible with living donor; History of severe cardiac disease (e.g., New York Heart Association [NYHA] Functional Class III or IV, myocardial infarction ≤6 months of randomization, ventricular tachyarrhythmias requiring ongoing treatment, unstable angina, or other significant cardiovascular diseases); Prior splenectomy; Has a known bleeding disorder; Has any active bacterial or other infection which is clinically significant in the opinion of the Investigator and is a contraindication to transplantation; Has participated in any other investigational drug study or was exposed to an investigational drug or device within 30 days of Screening; Has received rituximab (Rituxan®)≤3 months prior to Screening; Has received bortezomib (Velcade®)≤3 months prior to Screening; Has received alemtuzumab (Campath®)≤6 months prior to Screening; Hypersensitivity to murine proteins or to one of the product excipients; History of drug or alcohol abuse within the previous year; Unresolved meningococcal disease; Unresolved bacterial or fungal infection; Active infection with hepatitis B (HBV), hepatitis C (HCV) or human immunodeficiency virus (HIV); Pregnancy or lactation; Current cancer or a history of cancer within the 5 years prior to Screening with the exception of patients who have successfully treated nonmetastatic basal or squamous cell carcinoma of the skin; carcinoma in situ of the cervix; breast carcinoma in situ or other in situ lesion determined to be cured by removal; Any medical condition that, in the opinion of the Investigator, might interfere with the patient's participation in the study, poses an added risk for the patient, or confounds the assessment of the patient.

### Patient Withdrawal Criteria

Patients were informed that they had the right to withdraw from the study at any time for any reason without prejudice to their medical care. Patients were withdrawn from the study for any of the following reasons: patient request; patient was unwilling or unable to comply with the protocol; medical reason, at the discretion of the Investigator and/or the Medical Monitor. The reasons for patient study drug and/or patient withdrawal was recorded in the patient's CRF and in the source records. Patients who withdrew from the study after transplant completed the tests and evaluations scheduled for the final visit of the study. When a patient discontinued due to an adverse event (AE), the event was followed until it was resolved or in the opinion of the Principal Investigator the patient was determined to be medically stable. Every effort was made to undertake protocol-specified safety follow-up procedures. Patients who failed to return for final assessments were contacted by the site study staff in an attempt to have them comply with the protocol. As it was vital to obtain follow-up data on any patient withdrawn because of an AE or SAE, follow-up due diligence documentation will consisted of 2 phone calls followed by 1 registered letter to the patient's last known address. In any case, every effort was made to undertake protocol-specified safety follow-up procedures. Patients who discontinued participation in the study for reasons unrelated to the study or study drug (e.g., withdraw consent, lost during follow-up) were replaced as required for the study to meet its objectives. Replacement patients were assigned a unique identification number.

### Treatment of Patients

### Post-Transplant Immunosuppression and Concomitant Medications

Patients who underwent randomization and received their kidney transplants were required to take immunosuppressive and prophylactic medications to maintain allograft function and protect them from infection. In addition, medications were used to manage co-morbid conditions such as hypertension, hyperlipidemia, diabetes, and pain. These conditions were managed according to the SOC practices at the individual investigative sites.

Among the medications that were given to transplant recipients were:

### Induction Therapy:

Thymoglobulin (1.5 mg/kg×4 doses [6 mg/kg recommended, may use up to 7.5 mg/kg])

### Maintenance Immunosuppression:

Tacrolimus; Maintain trough levels at 4 to 11 ng/mL for Months 1 through 12. No calcineurin inhibitor avoidance or withdrawal protocols were allowed. Mycophenolate mofetil (MMF; Cellcept)/Enteric-coated mycophenolic acid (EC-MPA; Myfortic); MMF: 1 gram BID (may titrate down or alter dosing schedule for patient intolerance); EC-MPA: 720 mg BID (was titrated down or altered dosing schedule for patient intolerance); generic formulations of the above were acceptable for purposes of the study; prednisone per Transplant Center SOC but tapered to 5 mg daily by 3 months post transplantation; no steroid avoidance or withdrawal protocols was allowed.

Induction and Maintenance Immunosuppressive Therapies, should be Used Uniformly Across all Centers and in Both Arms of the Study.

### Infectious Disease:

The following were recommended: pneumococcal vaccine unless contraindicated, peritransplant prophylaxis against PCP, CMV, and fungus, and ID medicine advice regarding peritransplant antibiotic coverage. Whichever routine infectious disease prophylactic therapies was adopted by a Center was used uniformly within that Center throughout the course of the study and recorded in the CRF.

### Restricted Medications/Treatments:

The following medications/treatments were restricted as their use may have compromised the findings or interact with eculizumab except as outlined below: use of alemtuzumab (Campath®)≤6 months prior to Screening and post transplantation for both arms of the study; use of basiliximab (Simulect®) induction therapy for both arms of the study; use of bortezomib (Velcade®)≤3 months prior to Screening and post transplantation for both arms of the study. Bortezomib may have been used at the discretion of the Principal Investigator for salvage therapy of AMR not responsive to first line therapy; use of rituximab (Rituxan®)≤3 months prior to Screening and post transplantation for both arms of the study; Rituximab may have been used at the discretion of the Principal Investigator for salvage therapy of AMR not responsive to first line therapy; use of immunoadsorption at any time (in place of plasmapheresis); use of prophylactic PP or IVIg during the first 9 weeks post transplantation in the eculizumab treatment arm

### DSA and Cell-Based Crossmatch Evaluations

Patients underwent routine post transplantation monitoring for circulating DSA and cell-based cross match (XM) evaluations as follows: per protocol monitoring of DSA (Luminex LabScreen) and cell-based cross matches which include BFXM and/or TFXM were performed by the Central Laboratory at Days 0, 1, 7, 14, 21, 28, Week 9, and Months 3, 6, and 12. The Central Laboratories were blinded to the patients' treatment; DSA, BFXM, and TFXM tests were also collected at Month 36, but were not included in the primary efficacy analysis. They were sent to the Central Laboratory and used for purposes of long term follow up only; duplicate samples were sent to the Transplant Center's Local Laboratory for DSA and/or cell-based XMs (if available) to facilitate patient management. The Central Laboratory data was not used for patient management. Interim samples for patient management were analyzed at the Transplant Center's HLA Local Laboratory and may include any of the following tests: DSA, CDC, BFXM, and TFXM (if available). Duplicate samples were not required for the Central Laboratory.

### Treatment Compliance

Patients in the eculizumab treatment arm were administered eculizumab IV in a controlled setting such as a hospital, outpatient clinic or short-stay care unit, thereby ensuring compliance with study drug administration under the supervision of the Investigator. Study coordinators at the investigative sites ensured that all study participants were adequately informed on the specific treatment regimens required for compliance with the study protocol.

### Randomization and Blinding

Patients who met the inclusion/exclusion criteria, underwent desensitization therapy, and were cleared for transplantation by the Principal Investigator, were randomized to either eculizumab treatment or SOC prior to kidney transplantation. The randomization scheme ensured that patients were equally assigned on a 1:1 basis to the eculizumab treatment and SOC arms of the study. In addition, patients were stratified by the pre-transplant desensitization protocol that was used: PP and IVIg; PP alone; and IVIg alone.

This was an open label study and therefore, treatment assignment was not blinded to the Investigators. However, the central pathologists were blinded to patient identifiers and their treatment regimen.

### Study Drug Materials and Management

Study Drug:

Eculizumab was supplied in 30 mL vials with a solution concentration of 10 mg/mL. Each single entry 30 mL vial contained a solution concentration of 10 mg/mL and had enough solution to withdraw the indicated 30 mL.

Study Drug Packaging and Labeling:

The study drug eculizumab was released to the site upon receipt of all required essential documents based upon federal, state, and local regulations. Each kit had a single panel label describing the contents and a place for the pharmacist to record the patient number and initials. The pharmacy should immediately notify the distributor if vials were damaged. Eculizumab was stored in a secure, limited-access storage area.

Study Drug Storage:

The study drug (eculizumab) vials were stored in the original carton until time of use under refrigerated conditions at 2-8° C. (36-46° F.) and protected from light. The vials were not used after the expiration date stamped on the carton.

Study Drug Preparation:

Infusions of the study drug was prepared using aseptic technique. Each vial of eculizumab contained 300 mg of active ingredient in 30 mL of product solution. Eculizumab was diluted to a final admixture concentration of 5 mg/mL using the following steps: withdrew the required amount of eculizumab from the vial into a sterile syringe; transferred the recommended dose to an infusion bag; diluted eculizumab to a final concentration of 5 mg/mL by adding the appropriate amount (equal volume of diluent to drug volume) of 0.9% Sodium Chloride Injection, USP; 0.45% Sodium Chloride Injection, USP; 5% Dextrose in Water Injection, USP; or Ringer's Injection, USP to the infusion bag. The final admixed eculizumab 5 mg/mL infusion volume was 120 mL for 600 mg doses, 180 mL for 900 mg doses or 240 mL for 1200 mg doses

TABLE 9
Eculizumab and Diluent Volumes

| Eculizumab | Volume of | | Total Volume of |
|---|---|---|---|
| Dose | Eculizumab | Volume of Diluent¹ | Administration |
| 600 mg | 60 mL (2 vials) | 60 mL | 120 mL |
| 900 mg | 90 mL (3 vials) | 90 mL | 180 mL |
| 1200 mg | 120 mL (4 vials) | 120 mL | 240 mL |

¹One of the following diluents: a) 0.9% sodium chloride; b) 0.45% sodium chloride; c) 5% dextrose in water; d) Ringer's injection.

The infusion bag containing the diluted eculizumab solution was gently inverted to ensure thorough mixing of the product and diluent. Empty vials and vials with residual materials were kept for inspection by the study monitor prior to their destruction, or handled per local site pharmacy standard operating procedures for clinical study drugs.

Prior to administration, the admixture was allowed to adjust to room temperature (18-25° C., 64-77° F.). The admixture was not heated in a microwave or with any heat source other than ambient air temperature. The eculizumab admixture was inspected visually for particulate matter and discoloration prior to administration.

Administration and Stability of Solution

The eculizumab admixture was administered by IV infusion over 35 minutes (range 35-45 minutes). It was not necessary to protect the infusion bags from light while study drug was being administered to the patient. At the site's discretion, the diluted study drug was administered via gravity feed, a syringe-type pump, or an infusion pump. The patients were monitored for 1 hour following infusion.

Admixed solutions of eculizumab are stable for 24 hours at 2-8° C. (36-46° F.) and at room temperature. If the eculizumab was prepared more than 4 hours in advance of a patient's visit, the diluted material was stored at 2° C. to 8° C.

If an AE occurs during the administration of the study drug, the infusion was slowed or stopped at the discretion of the Investigator, depending upon the nature and severity of the event. The adverse event was captured in the patient's source document and CRF.

### Study Drug Accountability

The current International Conference on Harmonization (ICH) Good Clinical Practice (GCP) Guidelines required the Principal Investigator to ensure that study drug deliveries from the Sponsor were received by a responsible person (e.g. pharmacist). In addition, the following guidelines were also adhered to: study drug deliveries were recorded; study drug was handled and stored safely and properly; study drug was only dispensed to patients in accordance with the protocol; unused study drug was returned to the Sponsor or standard procedures for the alternative disposition of unused study drug were followed.

When a study drug shipment was received at the site, the pharmacist verified the contents, signed the packing invoice provided with the shipment, and maintained the original copy for review by the study monitor. A signed copy was faxed to the contact provided on the packing list and the duplicate copy kept in the pharmacy binder.

Accountability logs and Inventory logs were provided to assist the pharmacist in maintaining current and accurate inventory records covering receipt, dispensing, and disposition of the study drug. During the study, the following information was noted in the accountability log: the patient number(s), initials of patient(s) to whom drug was dispensed, kit number, the date(s) and time that the study drug was prepared and dispensed, and the initials of the pharmacist or designee who prepared the study drug. Sites were kept a running total of their drug supply. Empty vials and vials with residual materials were kept for inspection by the study monitor prior to their destruction, or handled per local site pharmacy standard operating procedures for clinical study drugs.

The study monitor examined the inventory during the study. Additionally, the inventory records were readily available to regulatory authorities, the local regulatory agency, or an independent auditor's inspection at any time.

### Study Drug Handling and Disposal

Drug inventory and accountability records for the study drug were kept by the Investigator/Pharmacist. Study drug accountability throughout the study was documented. The following guidelines were followed: the Investigator agreed not to supply study drugs to any person except the patients of the study; the Investigator/Pharmacist kept the study drug in a pharmacy or other locked and secure storage facility under controlled storage conditions, accessible only to those authorized by the Investigator or designee to dispense the investigative drug; a study drug inventory was maintained by the Investigator/Pharmacist; the inventory included details of material received and a clear record of when they were dispensed and to which patient.

At the conclusion or termination of the study, the Investigator/Pharmacist conducted a final drug supply inventory and to recorded the results of the inventory on the drug accountability record. Delivery records and records of used or returned study drug were reconciled. Appropriate forms of deliveries and returns were signed by the person responsible at the investigative site.

Used or unused study drug was destroyed at the study center according to standard institutional procedures after drug accountability had been conducted by the Sponsor or designee. A copy of the standard institutional procedure for destroying investigational drugs was provided to the Sponsor or designee upon request; unused study drug not destroyed at the site was returned to the Sponsor or designee at the end of the study or upon expiration.

### Assessment of Efficacy

### Kidney Allograft Biopsy Evaluations

All protocol and “for cause” kidney biopsies were processed and analyzed by the site's Local Pathology Laboratory. Processed slides and two paraffin embedded unstained slides were also forwarded to the Central Pathology imaging center for review by independent, blinded, pathologists.

Kidney biopsies were obtained under the following scenarios: for-cause allograft biopsy: biopsy was performed if there were clinical signs of allograft dysfunction based upon at least one of the following criteria, with or without elevation of DSA from baseline (day of transplant); a decrease in serum creatinine less than 10% per day in three consecutive days in the first week post transplantation compared to the Day 0 immediate post transplantation creatinine; an increase in serum creatinine of ≥30% from nadir. Nadir was defined as the lowest serum creatinine within the first week post transplantation; oliguria; clinical suspicion of AMR; protocol biopsy: mandated biopsies were performed at times as follow unless medically contraindicated: post reperfusion (intra-operative); day 14 post transplantation; month 3 post transplantation; month 12 post transplantation; month 36 post transplantation (for long term follow up only; will not be included in primary efficacy analysis); Protocol kidney biopsies were used to evaluate other secondary endpoints and also for evaluation of subclinical cases of AMR that were only evident on a histological basis. Protocol biopsies were read at the Transplant Center and used for clinical management. Slides that were read locally were sent to the Central Pathology Laboratory.

### Treatment of Antibody Mediated Rejection Episodes

The cumulative incidences of AMR at Week 9 and through Month 12 of the study were the primary and secondary endpoints respectively. The following guidelines were used in the treatment of AMR.

### For AMR Occurring During the Treatment Period Post Transplantation

Eculizumab Treatment Arm:

If the patient had a biopsy-proven (by local pathology) diagnosis of clinically significant (elevated creatinine by Local Laboratory) AMR during the first 9 weeks post transplantation, the patient was considered a treatment failure (See Criteria for Evaluation Section below for biopsy criteria). The patient received at least 3 treatment sessions of PP and/or IVIg for the treatment of AMR before it was determined by the Principal Investigator that the patient would remain on eculizumab, then supplemental doses of eculizumab were used as follows:

Eculizumab 600 mg (2 vials) was administered within 1 hour (Doses were given IV over 35-45 minutes) of completing each PP session and at least 1 hour before fresh frozen plasma (FFP) infusion or other protein replacement therapies. This was in order to maintain levels between 50 and 100 μg/mL of eculizumab, as had been predicted based on empirical experience and PK-PD modeling calculations for eculizumab under conditions of PP.

AMR was treated with eculizumab for at least 5 weeks or until the serum creatinine returns to within 10% of their pre-rejection baseline creatinine or until they achieve a new stable baseline serum creatinine defined as less than a 20% variation on three successive tests taken at least 24 hours apart. The maximum number of weeks that the patient was treated with eculizumab for acute AMR was 9.

SOC Control Arm:

If the patient had a biopsy-proven diagnosis (by local pathology) of clinically significant (elevated creatinine by Local Laboratory) AMR during the first 9 weeks post transplantation, the patient was considered a treatment failure. Patients diagnosed with AMR initially received PP and/or IVIg. Additional therapy (treatment of AMR after PP/IVIg therapy failure) were at the discretion of the Principal Investigator and may have included eculizumab. If eculizumab was used then it should be administered per the guidelines below (weeks were calculated from the day of first dose of eculizumab after AMR diagnosis): initial dose 1200 mg (Day 1), then; 900 mg weekly for 4 doses (Week 1), then; 900 mg weekly from 4 doses (Weeks 2, 3, and 4; +/−2 days), then; 1200 mg every other week beginning on Week 5 for Weeks 5, 7, and 9 (+/−2 days). Doses were given IV over 35-45 minutes.

If the patient continued to be treated with PP while receiving eculizumab then Eculizumab 600 mg (2 vials) was administered within 1 hour of completing each PP session and at least 1 hour before fresh frozen plasma (FFP) infusion or other protein replacement therapies. This was in order to maintain levels between 50 and 100 μg/mL of eculizumab, as had been predicted based on empirical experience and PK-PD modeling calculations for eculizumab under conditions of PP.

AMR was treated with eculizumab for at least 5 weeks or until the serum creatinine returns to within 10% of its pre-rejection baseline creatinine or until they achieved a new stable baseline serum creatinine defined as less than a 20% variation on three successive tests taken at least 24 hours apart. The maximum number of weeks that the patient was treated with eculizumab for acute AMR was 9.

Supplemental doses of eculizumab after PP was used as follows: Eculizumab 600 mg (2 vials) was administered within 1 hour of completing each PP session and at least 1 hour before fresh frozen plasma (FFP) infusion or other protein replacement therapies. This was in order to maintain levels between 50 and 100 μg/mL of eculizumab, had been predicted based on empirical experience and PK-PD modeling calculations for eculizumab under conditions of PP. Doses were given IV over 35-45 minutes.

For AMR Occurring after the Week 9 Treatment Period

AMR episodes occurring in either study arm after Week 9 were treated according to local SOC protocols and at the Principal Investigators' discretion (with the exception of prohibited medications). Eculizumab was used to treat AMR in either arm.

For the eculizumab treatment arm dosing was (weeks are calculated from the day of first dose of eculizumab after AMR diagnosis): initial dose 900 mg (Day 1), if dosed within 7 days of last dose of eculizumab; initial dose 1200 mg (Day 1), if dosed after 7 days of last dose of eculizumab; 900 mg weekly for 4 doses (Weeks 1), then; 900 mg weekly for 4 doses (Weeks 2, 3, and 4; +/−2 days), then; 1200 mg every other week beginning on Week 5 for Weeks 5, 7, and 9 (+/−2 days).

For the SOC control arm dosing was (weeks were calculated from the day of first dose of eculizumab after AMR diagnosis): initial dose 1200 mg (Day 1), then; 900 mg weekly for 4 doses (Week 1), then; 900 mg weekly for 4 doses (Weeks 2, 3 and 4; +/−2 days), then; 1200 mg every other week beginning on Week 5 for Weeks 5, 7, and 9 (+/−2 days).

### Assessment of Safety

### Data Monitoring Committee

An independent DMC was comprised of at least 3 clinicians experienced in high risk kidney transplantation. Since its primary function was to ensure patient safety, the DMC had access to all safety data and a data management expert was part of the DMC to ensure timely delivery of all required data. The DMC also had access to a statistician and/or an epidemiologist when necessary.

The broad remit of the DMC was to monitor safety and efficacy data as it was accumulated and to make decisions on study conduct and dose regimen to ensure patients' safety. The operational details and responsibilities of the DMC was specified in a charter.

### Safety Parameters

### Demographic/Medical History

The demographic information to be collected included date of birth, gender, race and/or ethnicity.

Medical history information to be collected included all ongoing conditions and relevant/significant medical history (including all major hospitalizations and surgeries). Symptoms related to renal transplantation and/or the underlying etiology of the disease was listed on the medical history form. Worsening of any of these signs or symptoms during the course of this study was captured as an AE.

The following vital signs were collected: body temperature (° C.), heart rate (beats/min), respiratory rate (breaths/min), and blood pressure (mmHg).Height (cm) and weight (kg) were collected at Screening. Post-Screening visits included weight collection only.

A complete physical exam consisting of an examination of the following: General Appearance, Skin, Head, Ears, Eyes, Nose and Throat (HEENT), Cardiovascular, Pulmonary, Abdomen/Gastrointestinal, Neurological, Lymph Nodes, Spine, Extremities, and Musculoskeletal. A genitourinary examination was performed unless a separate examination had been performed within 1 year by another physician and was documented in the patient record.

Abbreviated physical exams were completed at the time points specified on the Schedule of Assessments. The body systems included in these exams were based on Investigator judgment and/or patient symptoms.

A 12-lead ECG was performed. The data collected included heart rate, PR, QRS and QT intervals (corrected and uncorrected) and any abnormalities.

### Laboratory Assessments

### Hematology

The hematology panel included complete blood count (CBC), with differential and platelet counts. CBC includes red blood cells (RBCs), white blood cells (WBCs), hemoglobin, hematocrit, mean corpuscular volume (MCV), mean corpuscular hemoglobin (MCH) and mean corpuscular hemoglobin concentration (MCHC).

### Blood Chemistry Panel

The blood chemistries included: sodium, potassium, carbon dioxide, chloride, blood urea nitrogen, creatinine, glucose, calcium, magnesium, phosphorus, alkaline phosphatase, alanine aminotransferase (ALT), aspartate aminotransferase (AST), gamma-glutamyl transferase (GGT), lactic dehydrogenase (LDH), total and direct bilirubin, total protein, albumin, uric acid, and total cholesterol.

### Coagulation

The coagulation testing included an activated partial thromboplastin time (aPTT), Prothrombin Time (PT), international normalized ratio (INR), and fibrinogen and/or fibrinogen split products.

### Urinalysis

Urinalysis testing included protein, glucose, ketones, occult blood, and WBCs by dipstick, with microscopic examination and spot urine for urine protein/creatinine ratio.

### Pregnancy Screen

At Screening, a pregnancy test (serum beta-hCG) was completed for all females of child bearing potential.

### Adverse and Serious Adverse Events

### Adverse Event

An AE was defined as any untoward medical occurrence in a patient enrolled into this study regardless of its causal relationship to study treatment. Patients were instructed to contact the Principal Investigator or Sub-Investigator if any symptoms developed at any time after the informed consent had been signed. If there was any doubt as to whether or not a clinical observation was an AE, the event should be recorded and reported.

A treatment-emergent AE (TEAE) was defined as any event not present prior to exposure to Investigational Product or any event already present that worsens in either intensity or frequency following exposure to Investigational Product.

Adverse events were assigned Medical Dictionary for Regulatory Activities (MedDRA) preferred terms and tabulated as incidence rates per treatment group.

Safety evaluations consisted of monitoring and recording all adverse events, including SAEs, the regular monitoring of hematology, blood chemistry, coagulation parameters, and urine results. In addition, regular monitoring of vital signs, physical condition and body weight measurements was performed.

Patients were instructed to contact the Principal Investigator or Sub-Investigator if any symptoms developed at any time after the informed consent had been signed. If there was any doubt as to whether or not a clinical observation was an AE, the event should be recorded and reported.

### Serious Adverse Event

A serious adverse event was an AE occurring during any study phase (i.e., baseline, treatment, or follow-up), and at any dose of the investigational product, comparator or placebo, that fulfills one or more of the following: results in death; It is immediately life-threatening. The term “life-threatening” means that the patient was at risk of death at the time of the event. It does not refer to an event which hypothetically might have caused death if it were more severe. It requires in-patient hospitalization or prolongation of existing hospitalization. It results in persistent or significant disability or incapacity. Results in a congenital abnormality or birth defect.

Important medical events that did not result in death, but were life-threatening, or required hospitalization were considered an SAE when, based upon appropriate medical judgment, they jeopardized the patient and may have require medical or surgical intervention to prevent one of the outcomes listed in this definition. Examples of such medical events include allergic bronchospasm requiring intensive treatment in an emergency room or at home, blood dyscrasias or convulsions that did not result in patient hospitalization or the development of drug dependency or drug abuse.

### Other Adverse Events of Interest

Other adverse events of interest were identified by the Drug Safety Physician and also by the Clinical Study Team Physician during the evaluation of safety data for the Clinical Study Report. Significant AEs of particular clinical importance, other than SAEs and those AEs leading to discontinuation of the patient from the study, were classified as other AEs of interest. For each other adverse event of interest, a narrative was written and included in the Clinical Study Report.

Other Adverse Events of Interest for this study will included: Cumulative incidence of clinically significant infection (confirmed by culture, biopsy, genomic, or serologic findings) that required hospitalization or anti-infective treatment, or was otherwise deemed significant by the Investigator; cumulative incidence of CMV disease; cumulative incidence of BK virus disease; cumulative incidence of encapsulated bacterial infections; cumulative incidence of PTLD (post-transplant lymphoproliferative disease); cumulative incidence of malignancy; cumulative incidence of biopsy-proven acute cellular rejection that meets Banff 2007 criteria of any grade; proportion of patients that develop severe acute cellular rejection that do not respond to thymoglobulin or other lymphocyte depleting agents; cumulative incidence of allograft loss for reasons other than AMR; and overall patient survival.

### Relationship to Study Drug

An Investigator or designee who was qualified in medicine made the determination of relationship to the investigational product for each AE (Unrelated, Unlikely, Possible, Probable, or Definite). Unrelated: This relationship suggested that there was no association between the Investigational Product and the reported event; Unlikely: This relationship suggested that the clinical picture was highly consistent with a cause other than the Investigational Product but attribution could not be made with absolute certainty and a relationship between the Investigational Product and AE cannot be excluded with complete confidence; Possible: This relationship suggested that treatment with the Investigational Product was possibly caused or contributed to the AE, i.e. the event followed a reasonable temporal sequence from the time of drug administration and/or follows a known response pattern to the Investigational Product, but could also have been produced by other factors; Probable: This relationship suggested that a reasonable temporal sequence of the event with the Investigational Product administration exists and the likely association of the event with the Investigational Product. This was based upon the known pharmacological action of the Investigational Product, known or previously reported adverse reactions to the Investigational Product or class of drugs, or judgment based on the Principal Investigator's clinical experience. Definite: Temporal relationship to the Investigational Product, other conditions (concurrent illness, concurrent medication reaction, or progression/expression of disease state) did appear to explain event, corresponds with the known pharmaceutical profile, improvement on discontinuation, re-appearance on re-challenge.

### Recording Adverse Events

Adverse events spontaneously reported by the patient and/or in response to an open question from the study personnel or revealed by observation were recorded during the study at the investigational site. Clinically significant changes in laboratory values, blood pressure, and pulse needed to be reported as AEs. Abnormal values that constituted an SAE or lead to discontinuation of administration of study drug must have been reported and recorded as an AE. Any abnormal test findings that were considered adverse events or serious adverse events; investigators were strongly encouraged to report the diagnosis, sign or symptom instead of just the abnormal result. All non-serious adverse events were entered into the electronic case report form within 2-4 weeks and prior to the next scheduled monitoring visit.

Information about AEs were collected from the signing of the ICF. SAE information was collected from signing of ICF until the end of the study. The medical term for the AE was reported in standard medical terminology when possible. For each AE, the Investigator evaluated and reported the onset (date and time), resolution (date and time), intensity, causality, action taken, serious outcome (if applicable), and whether or not it caused the patient to discontinue the study.

Intensity was assessed according to the following scale: mild (awareness of sign or symptom, but easily tolerated); moderate (discomfort sufficient to cause interference with normal activities); severe (incapacitating, with inability to perform normal activities and may require systemic drug therapy or other treatment)

It is important to distinguish between serious and severe AEs. Severity is a measure of intensity. An AE of severe intensity may not be considered serious.

If it became known during the administration of the study drug that a patient was pregnant, the study drug was stopped immediately. In addition, for any woman who became pregnant at any time during the study, Pharmacovigilance was notified. Pharmacovigilance supplied the Investigator with a copy of a “Pregnancy Reporting and Outcome Form/Breast Feeding.” The patient was followed until the outcome of the pregnancy was known (spontaneous miscarriage, elective termination, normal birth or congenital abnormality), even if the patient was discontinued from the study. When the outcome of the pregnancy became known the form was completed and returned to Pharmacovigilance. If additional follow-up was required, the Investigator requested to provide the information.

Pregnancy in itself was not regarded as an AE unless there was a suspicion that an investigational product may have interfered with the effectiveness of a contraceptive medication.

All reports of congenital abnormalities/birth defects are SAEs. Spontaneous miscarriages were reported and handled as SAEs. Elective abortions without complications were not be handled as AEs.

The Investigator was responsible for reporting all AEs and SAEs observed or reported during the study regardless of their relationship to the study drug or their clinical significance.

All AEs that occur after the patient had given consent were reported in detail in the patient's source/chart and on the appropriate CRF and followed to satisfactory resolution or until the Principal Investigator or Sub-Investigator deems the event to be chronic or the patient to be stable. The description of the AE included the onset date, resolution date, intensity, seriousness, and the likelihood of relationship of the AE to the study drug.

Additional information reported included any required treatment or evaluations, and outcome. All reported AEs were followed to adequate resolution. Any medical condition that was present at the time that the patient was screened but did not deteriorate was not reported as an AE. However, deterioration at any time during the study was recorded as an AE.

### Statistics and Data Analysis

### General Considerations for Data Analysis

For continuous data, the mean, standard deviation, median, minimum and maximum was reported. For categorical data, percent and frequency was reported.

### Missing Data

Missing data on demographic, recipient-, donor-, and transplant-related information and on laboratory data was treated as missing; no method for imputation was planned. Missing data on time to event endpoints had events coded as right censored per the following table:

TABLE 10
Missing Data Events Coding for Time to Event Data Analyses

| Endpoint | Right Censoring |
|---|---|
| Time to First Biopsy- | Patients who did not experience a biopsy-proven |
| proven AMR | AMR at any time during follow-up will be right |
| | censored as of the date of last patient contact. |
| Time to First Biopsy- | Patients who did not experience a biopsy-proven |
| proven ACR | ACR at any time during follow-up will be right |
| | censored as of the date of last patient contact. |
| Graft Survival | Patients who are alive with functioning graft will |
| | be right censored as of the date of last patient |
| | contact |
| Patient Survival | Patients who are still alive as of the last known |
| | follow-up will be right censored as of the date of |
| | last patient contact |

### Analysis Datasets

One analysis set was defined; the safety set. The safety set comprised all patients who are randomized and transplanted.

All safety and efficacy analyses was performed using the safety set.

Because of the extensive Screening and desensitization procedures conducted between enrollment and transplantation, safety data was collected and summarized separately for any patients enrolled, but not transplanted.

### Efficacy Analysis

The primary analysis of all endpoints occurred after all patients had reached Month 12 post transplantation. Patients continued to be followed on Months 18, 24, and 36 for collection of additional follow up data on patient and graft survival, kidney disease, and disease status.

### Primary Efficacy Variable and Analysis

The primary efficacy composite endpoint was the Week 9 post transplantation treatment failure rate defined as the occurrence of 1) biopsy-proven AMR, 2) graft loss, 3) patient death, or 4) loss to follow-up.

The diagnosis of AMR was based on kidney allograft dysfunction and biopsy performed “for cause.” The histological diagnosis was based on Banff 2007 criteria for AMR as determined by the Central Pathology Laboratory. For this study only level II and level III AMR were accepted as defined below: presence of circulating anti-donor specific antibodies, morphologic evidence of acute tissue injury, such as (Type/Grade): Banff 2007 Level II—Capillary and/or glomerular inflammation (ptc/g >0) and/or thromboses; Banff 2007 Level III—Arterial—v3.

The primary efficacy variable was a binary outcome variable where patients meeting the above composite endpoint definition were considered treatment failures and all others were considered treatment successes. The observed difference in the incidence of treatment failure at 9 weeks post transplantation between the eculizumab treated group and the SOC control treated group were calculated along with a 95% confidence interval (CI) for the treatment difference. Although this was a phase II clinical study, test of the null hypothesis that the true treatment difference (π_{T}−π_{C}) was 0 was performed using the Cochran-Mantel-Haenszel test, stratified by pre-transplant desensitization protocol (See Sample Size and Power Considerations).

### Secondary Efficacy Variables and Analyses

Secondary efficacy endpoints for this study included the following: cumulative incidence of AMR that occurred between Week 9 and Month 12 post transplantation (AMR of any grade that meets Banff 2007 criteria); treatment failure rate defined as the occurrence of 1) biopsy-proven AMR; 2) graft loss; 3) patient death; or 4) loss to follow-up at Month 12 post transplantation; graft and patient survival at Months 6 and 12 post transplantation; histological evidence of AMR on protocol biopsies without other clinical findings at Day 14, and Months 3 and 12 post transplantation; overall pathological changes including chronic AMR, on protocol biopsies at Day 14, and Months 3 and 12 post transplantation; cumulative number of PP treatments at 12 months post transplantation; cumulative incidence of patients requiring splenectomy at 12 months post transplantation; incidence of delayed graft function (DGF) post transplantation (defined as the requirement for dialysis within the first post transplantation week for reasons other than post-operative hyperkalemia, acute pulmonary edema or fluid overload due to comorbid conditions); cumulative incidence and duration of dialysis between 7 days and 12 months post transplantation; renal function between Week 4 and Month 12 post transplantation as measured by: estimated Glomerular Filtration Rate (calculated) by Modification of Diet in Renal Disease 7 (MDRD7); serum creatinine defined as the value on at least 3 consecutive measurements taken at least 2 days apart while not on PP or dialysis that vary ≤20%.

Patient and graft survival, the cumulative incidence of delayed AMR, the cumulative incidence of biopsy-proven AMR without other clinical findings, and the cumulative incidence of biopsy-proven severe acute cellular rejection requiring treatment with lymphocyte depleting agents, each at the times post transplantation listed above, were estimated using the product-limit (Kaplan-Meier) method and compared between treatment groups using the z-statistic, stratified by pre-transplant desensitization protocol. In addition to point estimates, 95% CIs for rate estimates and rate differences between treatment groups were provided.

The Incidence of treatment failure rate at month 12 post transplantation was provided for each treatment group along with 95% CIs. Treatment groups will be compared using the Cochran-Mantel-Haenszel test, stratified by pre-transplant desensitization protocol.

The cumulative number of PP treatments post-transplantation was compared between treatment groups using the proportional odds model, stratified by pre-transplant desensitization protocol. Odds ratio (eculizumab versus SOC control) and 95% CI were provided as measure of strength of association and precision respectively.

The incidence of treatment of AMR diagnosed solely on histological evidence on protocol biopsies were provided for each treatment group along with 95% CIs. Treatment groups were compared using the Cochran-Mantel-Haenszel test, stratified by pre-transplant desensitization protocol. The actual treatments used were summarized or listed.

The percentage of patients requiring splenectomy, the incidence of DGF, and the incidence of dialysis beyond 7 days post transplantation were provided for each treatment group along with 95% CIs. Treatment groups were compared using the Cochran-Mantel-Haenszel test, stratified by pre-transplant desensitization protocol.

The duration of dialysis beyond 7 days post transplantation, and the number of days that serum creatinine was more than 30% above nadir following the diagnosis of AMR, were compared between treatment groups using an ANOVA of ranked data, including pre-transplant desensitization protocol in the model.

Overall pathological changes, including chronic AMR found on protocol biopsies at Day 14, and Months 3 and 12, and change in renal function between Week 4 and Month 12, were compared between treatment groups using mixed effects linear regression, including pre-transplant desensitization protocol in the model.

### Subgroup Analyses

Subgroup analyses were performed to explore whether there were any differences in outcomes related to the based upon CDC status (historical or current) and antibody level test results upon enrollment as determined by Luminex LabScreen.

### Safety Analysis

Safety assessments consisted of summarizing all AEs, including SAEs, hematology, blood chemistry and urine results, regular monitoring of vital signs, physical condition, and body weight measurements.

All AEs (serious and non-serious), regardless of relationship to study drug, were classified by system organ class and preferred term using the MedDRA (version 10.1 or higher). Incidence rates within each treatment group were tabulated for each system organ class and preferred term.

In addition to the above, the following specific safety assessments were summarized for each treatment group at Week 9 and Month 12 post transplantation: cumulative incidence of clinically significant infection (confirmed by culture, biopsy, genomic or serologic findings) that requires hospitalization or anti-infective treatment, or is otherwise deemed significant by the Investigator; cumulative incidence of CMV disease (incidence and %); cumulative incidence of BK virus disease (incidence and %); cumulative incidence of encapsulated bacterial infections; cumulative incidence of PTLD; cumulative incidence of malignancy; cumulative incidence of biopsy-proven acute cellular rejection that meets Banff 2007 criteria of any grade; proportion of patients that develop severe acute cellular rejection that do not respond to thymoglobulin or other lymphocyte depleting agents; cumulative incidence of allograft loss for reason other than AMR; and overall patient survival.

### Interim Analysis:

No formal statistical interim analyses of the primary and secondary efficacy variables were planned.

### Long Term Outcomes Data Collection

For purposes of long term follow up data collection to evaluate interim rejection episodes, graft loss, patient survival, kidney disease and disease status, all patients were seen at Months 18, 24, and 36. The following information was collected and summarized: chemistry panel (including BUN and sCr); tacrolimus trough levels; other Immunosuppressive levels; DSA, BFXM, and TFXM (Month 36 only); kidney allograft biopsy (Month 36 only). These data were not considered as part of the primary efficacy analysis.

### Sample Size and Power Considerations

The primary efficacy composite endpoint was the Week 9 post transplantation treatment failure rate defined as the occurrence of 1) biopsy-proven AMR, 2) graft loss, 3) patient death, or loss to follow-up. Sample size and power considerations were based on the primary efficacy variable with the following assumptions: composite endpoint treatment failure rate at Week 9 post transplantation in the SOC control group, π_{C}=36.3% (See Table 14 below); composite endpoint treatment failure rate at Week 9 post transplantation in the eculizumab treated group, π_{T}=10% (51); Null hypothesis, H₀: θ=π_{T}−π_{C}=0; alternative hypothesis, H₁::θ=π_{T}−π_{C}<−36.3%; type I error, α=0.05 (two-sided significance test); statistical test=Fisher's Exact test; randomization ratio=1:1.

Although the primary efficacy endpoint analysis was performed using the Cochran-Mantel-Haenszel test, Fisher's exact test was used for the sample size calculation since the relationship of the strata on the outcome was unknown. A Fisher's Exact test with a 0.050 two-sided significance level had >80% power to detect the difference between a control group failure rate of π_{C}=0.363 and a treatment failure rate of π_{T}=0.100 when the sample size in each group was 45.

The background rate of treatment failure (primary efficacy variable) of 36.3% was derived from a pooled analysis of AMR incidence obtained from the literature (See Table 14) and assuming 40 patients will be enrolled into the protocol under the original protocol inclusion criteria and ⅓ of patient will still meet the original criteria once the amendment is implemented. Using a random effects model we found that the background rate of AMR was 34.8% (95% CI=26.3%−44.3%); which was increased to 40% to take into account uncertainty regarding graft loss and patient death at 9 weeks post transplantation as part of the composite endpoint. This was felt to provide a more conservative estimate of the background rate of treatment failure at 9 weeks post transplantation. For patients with a positive complement-dependent cytotoxicity (CDC) cross match (current or historic) or a positive B-cell flow cross match (BFXM) and/or T-cell flow cross match (TFXM) we expect the failure rate to decrease to 30% in the SOC arm. The sample size calculation assumes 40 patients will be enrolled into the protocol under the original protocol inclusion criteria and once the amendment is implemented approximately ⅓ of patient will still meet the original criteria.

A sub-study to evaluate the immune response to meningococcal vaccination was performed on 20 patients at selected centers.

TABLE 11
Background Rate of AMR in Desensitized Recipients of Living Donor
Kidney Transplant

| | Sample | | | |
|---|---|---|---|---|
| Citation | Size | AMR | AMR %¹ | Exact 95% CI |
| Thielke et al. | 51 | 12 | 23.50% | 12.8%-37.5% |
| Transplantation 87: | | | | |
| 268-273, 2009 | | | | |
| Stegall et al. ATC 2010: | 51 | 20 | 39.20% | 25.8%-53.9% |
| Abstract #1 American | | | | |
| Transplant Congress | | | | |
| 2010 | | | | |
| Magee et al. | 28 | 11 | 39.30% | 21.5%-59.4% |
| Transplantation 28: 96- | | | | |
| 103; 2008 | | | | |
| Stegall et al. Am J | 49 | 19 | 38.80% | 25.2%-53.8% |
| Transpl 6: 346-351; | | | | |
| 2006 | | | | |
| Vo et al. | 31 | 11 | 35.50% | 19.2%-54.6% |
| Transplantation 89: | | | | |
| 1095-1102; 2010 | | | | |
| | | | Pooled | Random |
| | | | Estimate | Effects CI |
| | | | 34.80% | 26.3%-44.3% |

¹Pooled estimates were derived using random effects model

List of Laboratory Tests

TABLE 12
Chemistry, Coagulation, Hematology, Urinalysis,
Pregnancy, and HLA Tests
Chemistry

| | | | |
|---|---|---|---|
| Sodium | Carbon | Total Cholesterol | AST |
| | Dioxide | | |
| Potassium | Albumin | Total Protein | ALT |
| Chloride | BUN | Creatinine | Alkaline Phosphatase |
| Calcium | Magnesium | Phosphorus | Glucose |
| Uric Acid | LDH | GGT | Total and Direct |
| | | | Bilirubin |

Coagulation

| | | |
|---|---|---|
| PT | aPTT | INR |

Fibrinogen/Fibrinogen Split Products
ab

| | | | |
|---|---|---|---|
| Hemoglobin | Hematocrit | RBC | WBC |
| MCV (mean | Mean | Mean Corpuscular | Platelets |
| corpuscular | Corpuscular | Hemoglobin | |
| volume) | Hemoglobin | Concentration | |
| | (MCH) | (MCHC) | |

Urinalysis with Microscopy

| | | |
|---|---|---|
| Protein | Ketones | WBC's by dipstick |
| Glucose | Occult Blood | Microscopy |

Spot Urine for Urine Protein/Creatinine Ratio
Pregnancy Testing (if applicable)
Serum beta-hCG
HLA Laboratory Testing:

Donor Specific Antibody Test - DSA
Complement Dependent Cytotoxicity - CDC
B-cell Flow Cross Match - BFXM
T-cell Flow Cross Match - TFXM

### Example 2: A Randomized, Open-Label, Multicenter Trial to Determine Safety and Efficacy of Eculizumab in the Prevention of AMR in Living Donor Kidney Transplant Recipients Requiring Desensitization Therapy

This trial enrolled 102 patients to an eculizumab (N=51) or SOC (N=51) arm. Patients included in the trial had been on the kidney transplant wait list a median of 3.4 years (ranging from 0.1 to 22.2 years).

The primary objective of this study was to evaluate the efficacy of eculizumab in the first 9 weeks as assessed by the primary endpoint: treatment failure rate, defined as the occurrence of 1) biopsy-proven AMR, 2) graft loss, 3) patient death, or 4) loss to follow up. The diagnosis of AMR for the determination of the primary efficacy endpoint was based on “for-cause” kidney biopsies. In addition, protocol biopsies are performed on all patients at predetermined time points.

The 9-week primary efficacy endpoint was assessed using Fisher's exact test. Assumptions for the power calculations for α=0.05 (two-sided), 80% power, control failure rate of 36.3% and treatment failure rate of 10%, 45 patients are required in each treatment arm. Considering that all patients are categorized as either treatment failures or treatment successes at Week 9 based on the defined primary endpoint, which includes loss to follow-up, no patient should have missing data for the primary endpoint. Two-sided hypothesis testing was used with a Type I error rate of 0.05 for all endpoints. No adjustment for multiplicity was needed as there is a single primary efficacy endpoint. A planned sensitivity analysis was conducted to explore the effects of local vs. central pathology results on the primary efficacy endpoint.

To date no efficacy analyses beyond those data associated with the primary endpoint have been conducted including 1-year outcomes.

### Biopsy Assessment

In this trial, biopsies evaluated by the Local pathologist at each site were the basis of treatment decisions. Locally read slides were also provided to Biomedical Systems, the central pathology imaging contract research organization (CRO) (either the exact same slides or digitized versions) who then provided digitized slides for review by two independent, blinded pathologists (Central pathology). Assessment of biopsy proven AMR as part of the composite primary endpoint was based on Central pathology. A single diagnosis was accomplished either through agreement between the two Central pathologists or when there was disagreement, through adjudication by a third blinded pathologist. There were 4 possible categorizations of acute AMR based on the reviews of the biopsies: Clean (no acute AMR), Grade I, Grade II, or Grade III. However, the primary endpoint of treatment failure included a binary assessment whereby clean and Grade I were categorized as “acute AMR=No” and Grade II and III cases were categorized as “acute AMR=Yes”. If the Central pathologist's outcome on a biopsy was Grade II for pathologist A and Grade III for Pathologist B, they were considered to be in agreement and no adjudication was required. Although Grade I cases were included in the “acute AMR=No” category, at the time this study was designed, there was very little clinical experience that supported the understanding of the histologic features of acute AMR. Thus, it was also not well appreciated that the Banff criteria were not limited to identifying the severe acute antibody-dependent complement-mediated rejection (SAACR) that this trial focused on. The fact that Grade I lesions on biopsy is a clinically significant diagnosis only became evident as clinical experience accumulated. AMR experts have noted that the Grade I cases of early acute AMR are clinically meaningful because early clinical diagnosis results in less time for the histological lesion to develop and these experts agree that if left untreated, Grade I acute AMR would be expected to progress further and result in similar outcomes to Grade II and III events. For this reason additional analyses below will include the evaluation of Grade I acute AMR in the analyses.

The data provided below include the primary endpoint based on the Central pathology as well as sensitivity analyses for the primary endpoint. In the original analysis of the primary endpoint there were 5 SOC patients with graft loss; this count is reduced by 1 in the current data, as an investigator later confirmed a data correction for a patient who did not actually have graft loss.

The results of the primary endpoint along with the individual components that comprise the composite measure are provided in Table 13. The treatment failure rates were not significantly different (p=0.760) between the eculizumab and SOC arms. There were a greater number of graft losses on SOC; however, the number of events was small and limits further interpretation. Of note, this study was powered based on expected treatment failure rates of 10% for eculizumab and 36.3% for SOC. The expected rate was observed for eculizumab but a much lower rate than expected was observed for SOC (13.7%).

TABLE 13
Primary Endpoint

| | | | Difference | P-value |
|---|---|---|---|---|
| | Eculizumab | SOC | (exact 95% | (Fisher's exact |
| Endpoint | (N = 51) | (N = 51) | CI) | test) |
| Treatment | 5 (9.8%) | 7 (13.7%) | −3.9% | 0.760 |
| Failure | | | (−23.9%, | |
| | | | 16.3%) | |
| AMR | 5 (9.8%) | 5 (9.8%) | | |
| Graft Loss | 1 (2.0%) | 4 (7.8%) | | |
| Death | 1 (2.0%) | 1 (2.0%) | | |
| Lost to | 0 (0%) | 0 (0%) | | |
| Follow-Up | | | | |

Several sensitivity analyses were carried out to better understand the data. Table 14 summarizes the analysis of the composite primary endpoint but using the biopsy results from the Local pathologists, a prespecified analysis outlined in the statistical analysis plan. Of note, the treatment failure rate based on Local pathology was different than that based on Central pathology with a larger number of cases of acute AMR being diagnoses, primarily in the SOC arm.

TABLE 14
Primary Endpoint using Local Pathology

| | | | Difference | P-value |
|---|---|---|---|---|
| | Eculizumab | SOC | (exact | (Fisher's |
| Endpoint | (N = 51) | (N = 51) | 95% CI) | exact test) |
| Treatment | 7 (13.7%) | 15 (29.4%) | −15.7% | 0.091 |
| Failure | | | (−35.1%, | |
| | | | 4.7%) | |
| AMR | 7 (13.7%) | 12 (23.5%) | | |
| Graft Loss | 1 (2.0%) | 4 (7.8%) | | |
| Death | 1 (2.0%) | 1 (2.0%) | | |
| Lost to Follow-Up | 0 (0%) | 0 (0%) | | |

Table 15 outlines a sensitivity analysis that includes Grade I AMR for both Central and Local pathology. Although not pre-specified for the primary endpoint, this analysis was conducted to better understand whether the differences between the Central and Local pathology may have been primarily due to potential differences in interpretation of Grade I and II cases. In addition, as noted above, AMR experts have noted that the Grade I cases of early acute AMR are clinically meaningful because early clinical diagnosis results in less time for the histological lesion to develop; left untreated, Grade I acute AMR would be expected to progress further and result in similar outcomes to Grade II and III events. Therefore, it was thought to be important to understand this component to best understand the treatment effect of eculizumab. As with the primary endpoint, a higher incidence of acute AMR was noted when the assessment was based on Local pathology and this difference occurred across grades I, II and III.

TABLE 15
Primary Endpoint including Grade I AMR,
Central and Local Pathology

| | | | | |
|---|---|---|---|---|
| | | | | P-value |
| | Eculizumab | SOC | Difference | (Fisher's |
| Endpoint | (N = 51) | (N = 51) | (exact 95% CI) | exact test) |

Results Based on Central Pathology

| | | | | |
|---|---|---|---|---|
| Treatment | 5 (9.8%) | 9 (17.6%) | −7.8% | 0.389 |
| Failure | | | (−27.7%, 12.5%) | |
| AMR | 5 (9.8%) | 7 (13.7%) | | |
| Graft Loss | 1 (2.0%) | 4 (7.8%) | | |
| Death | 1 (2.0%) | 1 (2.0%) | | |
| Lost to | 0 (0%) | 0 (0%) | | |
| Follow-Up | | | | |

Results Based on Local Pathology

| | | | | |
|---|---|---|---|---|
| Treatment | 10 (19.6%) | 21 (41.2%) | −21.6% | 0.031 |
| Failure | | | (−40.6%, -1.2%) | |
| AMR | 10 (19.6%) | 18 (35.3%) | | |
| Graft Loss | 1 (2.0%) | 4 (7.8%) | | |
| Death | 1 (2.0%) | 1 (2.0%) | | |
| Lost to | 0 (0%) | 0 (0%) | | |
| Follow-Up | | | | |

Given the large differences observed in the treatment failure rates between Central and Local pathology, an assessment of all pathology results was undertaken. The analysis encompassed all biopsies assessed over the 9-week primary endpoint period and included for-cause and per-protocol biopsies; a total of 241 biopsies were collected for this time period. The evaluation was by Grade of AMR assessing the overall Central pathology outcome as well as the results for the individual Central pathologists. As noted above, a single diagnosis resulted from the Central pathology evaluation based on either agreement between the two primary Central pathologists or adjudication by the third Central pathologist.

The Local and overall Central grading scores for each of the 241 biopsies evaluated are shown in Table 16, along with the kappa coefficient, which measures the level of agreement between the Local and overall Central grading scores, accounting for expected agreement by chance. A relatively large number (75.5%) of biopsies were assessed as “clean” (less than a Grade I AMR) by both the overall Central and the Local pathologists. However, when either the Local or overall Central pathology categorized a biopsy as Grades I, II, or III, there was generally poor agreement. The Kappa score for agreement overall was 0.225, which would generally be considered only fair or slight. The discordant biopsies included 4 that were assessed locally as clean but characterized as Grade II or III centrally and 21 biopsies assessed locally as Grade II or III and categorized as clean by the Central pathologists.

TABLE 16
Pathology AMR Grading Results: Local versus Central Pathology

| | | Central | | Central | |
|---|---|---|---|---|---|
| | | Grade I | Central | Grade III | |
| Local | Central Clean | AMR | Grade II AMR | AMR | Total |
| Clean | 182 (75.5%) | 0 | 3 (1.2%) | 1 (0.4%) | 186 (77.2%) |
| Grade I acute | 20 (8.3%) | 0 | 3 (1.2%) | 0 | 23 (9.5%) |
| AMR | | | | | |
| Grade II acute | 19 (7.9%) | 3 (1.2%) | 6 (2.5%) | 0 | 28 (11.6%) |
| AMR | | | | | |
| Grade III acute | 2 (0.8%) | 0 | 1 (0.4%) | 1 (0.4%) | 4 (1.7%) |
| AMR | | | | | |
| Total | 223 (92.5%) | 3 (1.2%) | 13 (5.4%) | 2 (0.8%) | 241 |

Total number of Biopsies are 241
Simple Kappa: 0.225
95% CI 0.111-0.338
Note:
the (percentages) above are the proportion of the total 241 biopsies categorically presented in each cell

Although there was a single Central pathology score for each biopsy for the primary endpoint, in order to better understand the discordance between the Local and Central pathology, a comparison of the data from the two Central pathologists was undertaken to determine the level of concordance between the Central pathologists. Similar to the comparisons between the Local and Central biopsy results in Table 17, there was relatively good agreement between the two Central pathologists when a biopsy was classified as clean (86.3% were noted as clean by both Central pathologists). However, similar to what was noted above for the Local versus Central pathology outcomes, there were numerous cases when a greater than 1 grade difference was noted between the two Central pathologists. This included 10 biopsies deemed clean by Central Reader B which were classified as Grade II (9 cases), and Grade III (1 case) by Reader A and 3 cases deemed clean by Reader A which were classified as Grade II by Reader B. As noted previously, in the cases of disagreement between the two primary Central readers (acute AMR=yes versus acute AMR=no), a review by the third Central reader responsible for adjudicating disagreements was used to gain consensus.

TABLE 17
Central Pathology AMR Grading Results: Individual Primary Pathologists

| | | | Reader A: | | |
|---|---|---|---|---|---|
| Central Reader | Reader A: | Reader A: | Grade II | Reader A: | |
| B | Clean | Grade I AMR | AMR | Grade III AMR | Total |
| Clean | 208 (86.3%) | 5 (2.1%) | 9 (3.7%) | 1 (0.4%) | 223 (92.5%) |
| Grade I acute | 2 (0.8%) | 0 | 5 (2.1%) | 0 | 7 (2.9%) |
| AMR | | | | | |
| Grade II acute | 3 (1.2%) | 1 (0.4%) | 5 (2.1%) | 0 | 9 (3.7%) |
| AMR | | | | | |
| Grade III acute | 0 | 0 | 1 (0.4%) | 1 (0.4%) | 2 (0.8%) |
| AMR | | | | | |
| Total | 213 (88.4%) | 6 (2.5%) | 20 (8.3%) | 2 (0.8%) | 241 |

Total number of Biopsies are 241
Simple Kappa: 0.372
95% CI 0.220-0.523
Note:
the (percentages) above are the proportion of the total 241 biopsies categorically presented in each cell

Based on the level of discordance noted above, the grades for the 241 biopsies were summarized by a cross-tabulation of three pathologists (one Local and the two Primary Central Pathologists) to understand the differences among them. Table 18 displays the results of this assessment.

Other than the biopsies agreed to as clean among all pathologists or between the two Central pathologists, there was little agreement among the diagnoses. Between Central pathologists, when a biopsy met the threshold for grade I, II, or III acute AMR by at least one of the pathologists, only 6 (18%) of the biopsies had an agreed diagnosis, while 27 (82%) did not. When considering the binary outcome of Clean plus Grade I, and Grade II plus Grade III, 14 (42%) biopsy assessments were in agreement, whereas 19 (58%) were not.

TABLE 18
Local and Central Acute AMR Grading Results (for-cause and per-
protocol): Comparison by Pathologist and Grade

| | Central | | | |
|---|---|---|---|---|
| Local | Reader A | Central Reader B | | |
| Acute AMR | Acute AMR | Acute AMR | | |
| Grading Result | Grading Result | Grading Result | Frequency | Percent |
| Clean | Clean | Clean | 178 | 73.9 |
| Clean | Grade I | Clean | 1 | 0.4 |
| Clean | Grade II | Clean | 3 | 1.2 |
| Clean | Grade II | Grade I | 2 | 0.8 |
| Clean | Grade II | Grade II | 1 | 0.4 |
| Clean | Grade III | Grade III | 1 | 0.4 |
| Grade I | Clean | Clean | 16 | 6.6 |
| Grade I | Clean | Grade II | 1 | 0.4 |
| Grade I | Grade I | Clean | 3 | 1.2 |
| Grade I | Grade II | Clean | 1 | 0.4 |
| Grade I | Grade II | Grade II | 2 | 0.8 |
| Grade II | Clean | Clean | 14 | 5.8 |
| Grade II | Clean | Grade I | 2 | 0.8 |
| Grade II | Clean | Grade II | 2 | 0.8 |
| Grade II | Grade I | Clean | 1 | 0.4 |
| Grade II | Grade I | Grade II | 1 | 0.4 |
| Grade II | Grade II | Clean | 3 | 1.2 |
| Grade II | Grade II | Grade I | 3 | 1.2 |
| Grade II | Grade II | Grade II | 2 | 0.8 |
| Grade III | Grade II | Clean | 2 | 0.8 |
| Grade III | Grade II | Grade III | 1 | 0.4 |
| Grade III | Grade III | Clean | 1 | 0.4 |

Note:
Bolded cells denote complete agreement between pathologists

Given these data, a group of experts in acute AMR including kidney transplant surgeons, transplant nephrologists, and pathologists were assembled to discuss the level of discordance. One aspect identified as an inconsistency between the information provided to the Local and Central pathologists was the amount of data the two groups had on each case to confirm or rule out a diagnosis of acute AMR. The protocol outlined that the diagnosis of acute AMR was to be based on kidney allograft dysfunction and biopsies performed “for cause” which included the presence of circulating DSAs, along with morphologic evidence of acute tissue injury based on the biopsy findings. However, only the Local pathologists had access to the clinical information such as allograft dysfunction and details including whether a biopsy was performed for-cause for each case. Further discussion with the experts as well as the Central pathologists revealed that in clinical practice, the diagnosis of acute AMR involves the assessment of these clinical components in addition to the pathological components.

Given this information, the decision was made to have the Central pathologists reassess a subset of the biopsies to determine if providing the clinical information on each case would result in greater concordance among the Central pathologists and how the reassessed biopsies would compare to the Local pathology diagnoses. In this reassessment, the pathologists reviewed the same biopsy slides that were provided by the imaging CRO and remained blinded to the treatment group.

Analyses based on blinded central biopsy reassessments using clinical Information available to local pathologists.

This section describes the process that was put in place in the reassessment of the Central biopsies and provides the revised biopsy data.

Because the main goal of the biopsy reassessment was to try to understand the underlying cause for the discordance among the pathologists, it was decided that details of the Banff criteria that are the basis of the acute AMR diagnosis and which were not part of the original electronic data capture should be documented. Having these data would allow Alexion to understand what components of the criteria might contribute to differences in diagnoses. Towards this goal, a spreadsheet was designed in collaboration with the Central pathologists that included all of the acute AMR criteria outlined in the Banff 2007; Solez K et al. Am J Transplant. 2008 April; 8(4):753-60), as set forth below in Table 19.

TABLE 19
Banff Diagnostic Categories for Renal Allograft Biopsies - Banff'07 Update^{1,2}

| 1. | Normal | |
|---|---|---|
| 2. | Antibody-mediated changes (may coincide with categories 3, 4 and 5 and 6) | |
| | Due to documentation of circulating antidonor antibody, and C4d³ or allograft pathology | |
| | C4d deposition without morphologic evidence of active rejection | |
| | C4d+, presence of circulating antidonor antibodies, no signs of acute or chronic TCMR or | |
| | ABMR (i.e. g0, cg0, ptc0, no ptc lamination). Cases with simultaneous borderline | |
| | changes or ATN are considered as indeterminate | |
| | Acute antibody-mediated rejection⁴ | |
| | C4d+, presence of circulating antidonor antibodies, morphologic evidence of acute tissue | |
| | injury, such as (Type/Grade): | |
| | I. | ATN-like minimal inflammation |
| | II. | Capillary and or glomerular inflammation (ptc/g > 0) and/or thromboses |
| | III. | Arterial - v3 |
| | Chronic active antibody-mediated rejection⁴ | |
| | C4d+, presence of circulating antidonor antibodies, morphologic evidence of chronic | |
| | tissue injury, such as glomerular double contours and/or peritubular capillary basement | |
| | membrane multilayering and/or interstitial fibrosis/tubular atrophy and/or fibrous | |
| | intimal thickening in arteries | |
| 3. | Borderline changes: ‘Suspicious’ for acute T-cell-mediated rejection (may coincide with | |
| | categories 2 and 5 and 6) | |
| | This category is used when no intimal arteritis is present, but there are foci of tubulitis (t1, | |
| | t2 or t3) with minor interstitial infiltration (i0 or i1) or interstitial infiltration (i2, i3) | |
| | with mild (t1) tubulitis | |
| 4. | T-cell-mediated rejection (TCMR, may coincide with categories 2 and 5 and 6) | |
| | Acute T-cell-mediated rejection (Type/Grade:) | |
| | IA. | Cases with significant interstitial infiltration (>25% of parenchyma affected, i2 or i3) |
| | | and foci of moderate tubulitis (t2) |
| | IB. | Cases with significant interstitial infiltration (>25% of parenchyma affected, i2 or i3) |
| | | and foci of severe tubulitis (t3) |
| | IIA. | Cases with mild-to-moderate intimal arteritis (v1) |
| | IIB. | Cases with severe intimal arteritis comprising >25% of the luminal area (v2) |
| | III. | Cases with ‘transmural’ arteritis and/or arterial fibrinoid change and necrosis of |
| | | medial smooth muscle cells with accompanying lymphocytic inflammation (v3) |
| | Chronic active T-cell-mediated rejection | |
| | ‘chronic allograft arteriopathy’ (arterial intimal fibrosis with mononuclear cell | |
| | infiltration in fibrosis, formation of neo-intima) | |
| 5. | Interstitial fibrosis and tubular atrophy, no evidence of any specific etiology | |
| | (may include nonspecific vascular and glomerular sclerosis, but severity graded by | |
| | tubulointerstitial features) | |
| | Grade | |
| | I. | Mild interstitial fibrosis and tubular atrophy (<25% of cortical area) |
| | II. | Moderate interstitial fibrosis and tubular atrophy (2650% of cortical area) |
| | III. | Severe interstitial fibrosis and tubular atrophy/loss (>50% of cortical area) |

| | |
|---|---|
| 6. | Other: Changes not considered to be due to rejection acute and/or chronic (for diagnoses |
| | see Table 14 in (42); may include isolated g, cg or cv lesions and coincide with categories 2, |
| | 3, 4 and 5) |

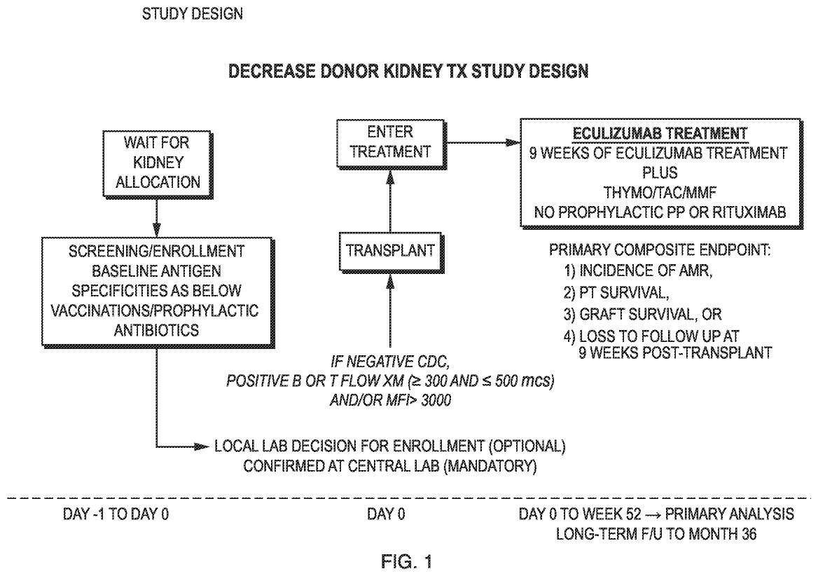

¹The 2007 updates are underlined.
²All existing scoring categories (g, t, v, i, cg, ct, ci, cv, ah, mm) remain unchanged (42)
³Please refer to Table 2 and FIG. 1.
⁴Suspicious for antibody-mediated rejection if C4d (in the presence of antibody) or alloantibody (C4d+) not demonstrated in the presence of morphologic evidence of tissue injury.

During the preparation of the spreadsheets, it was also discovered that the Central pathologists had changed one of the Banff criteria for their evaluation of the program biopsies. It was learned that the 3 central pathologists developed a consensus document among themselves prior to reading any of the biopsies from the study to help ensure they were all approaching the evaluation of the biopsies in a similar manner. This consensus document outlined that the diagnosis of acute AMR would require the following: capillaritis with neutrophils and/or mononuclear cells of level ptc 2 or greater, glomerulitis with neutrophils of g2 or greater, glomerular thrombi or fibrinoid necrosis of arteries/arterioles and C4d staining of level 2 or greater (>10%) by IHC (C4d2).

However, this definition stipulates greater requirements than the Banff criteria (and the protocol) which only require the presence of capillary and/or glomerular inflammation (ptc/g greater than 0) and/or thrombosis. In discussing this situation with the pathologists, they communicated that this change was made (without Alexion's permission or knowledge) to avoid “over-reading” the biopsies because they would not have the date of the biopsy relative to the date of transplant. Thus, the additional analyses conducted on the biopsy reassessments include clinical information on each case and a requirement that the Central pathologists strictly adhere to the Banff and protocol criteria.

The initial plan for the biopsy reassessment was to focus on all biopsies that were not unanimously categorized as “clean” by both Local and Central pathologists. There were 178 biopsies unanimously assessed as clean among all pathologists, leaving 63 biopsies to reassess that were not unanimously categorized as clean. An additional 37 biopsies unanimously assessed as “clean” were also chosen at random as internal controls, for a total of 100 biopsies (63 plus 37).

After learning about the greater requirement for peritubular capillaritis and glomerular inflammation applied to the original biopsies by the Central Pathologists, an additional 9 “clean” biopsies were added to the 100 biopsies. These last 9 biopsies, previously unanimously scored as clean, were chosen because they were “for cause” biopsies which were assessed as C4d positive by the Central evaluation. This assured that any biopsies that had the ability to influence the primary endpoint which was based only on “for cause” biopsies was included in the assessment. The Imaging CRO reloaded these 109 biopsies for the pathologists and provided the following clinical information on each case: Date of Biopsy (Number of days post transplant); Reason for biopsy; Primary cause of renal disease; Date of diagnosis of renal disease; Creatinine over time; DSA over time; Tacrolimus levels; Immunosuppressants; and List of Adverse Events of Infection.

The clinical details deemed important to aid in the diagnosis were identified by the Central pathologists. Results of additional analyses based on blinded central biopsy reassessments using clinical information available to local pathologists. The results of the biopsy reassessment are provided in the tables below. There were 132 biopsies considered “clean” by the Local and both Central pathologists that were not reassessed.

Table 20 summarizes the primary endpoint analysis using the data from the reassessed biopsies. The new analysis increased the number of patients with acute AMR by 1 and 4 patients in the eculizumab and SOC groups, respectively. The treatment failure rate in the eculizumab group was slightly more than one half of that observed in the SOC group; however, the difference was not statistically significant.

TABLE 20
Primary Endpoint: Biopsy Reassessment

| | | | Difference | P-value |
|---|---|---|---|---|
| | Eculizumab | SOC | (exact | (Fisher's |
| Endpoint | (N = 51) | (N = 51) | 95% CI) | exact test) |
| Treatment | 6 (11.8%) | 11 (21.6%) | −9.8% | 0.288 |
| Failure | | | (−29.6%, | |
| | | | 10.5%) | |
| AMR | 6 (11.8%) | 9 (17.6%) | | |
| Graft Loss | 1 (2.0%) | 4 (7.8%) | | |
| Death | 1 (2.0%) | 1 (2.0%) | | |
| Lost to Follow-Up | 0 (0%) | 0 (0%) | | |

Table 21 provides a comparison of the Primary Endpoint including Grade I AMR using the reassessed biopsy data for the Central pathology and the previous data for the Local pathology. Based on the reassessment, 1 and 6 additional acute AMR events were added to the eculizumab and SOC groups, respectively, and the difference between eculizumab and SOC for the treatment failure rate was statistically significant.

TABLE 21
Primary Endpoint including Grade I AMR; Central and Local
Pathology: Biopsy Reassessment

| | | | | |
|---|---|---|---|---|
| | | | Difference | P-value |
| | Eculizumab | SOC | (exact 95% | (Fisher's exact |
| Endpoint | (N = 51) | (N = 51) | CI) | test) |

Results Based on Central Pathology

| | | | | |
|---|---|---|---|---|
| Treatment | 6 (11.8%) | 15 (29.4%) | −17.7% | 0.048 |
| Failure | | | (−37.0%, | |
| | | | 2.7%) | |
| AMR | 6 (11.8%) | 13 (25.5%) | | |
| Graft Loss | 1 (2.0%) | 4 (7.8%) | | |
| Death | 1 (2.0%) | 1 (2.0%) | | |
| Lost to | 0 (0%) | 0 (0%) | | |
| Follow-Up | | | | |

Results Based on Local Pathology

| | | | | |
|---|---|---|---|---|
| Treatment | 10 (19.6%) | 21 (41.2%) | −21.6% | 0.031 |
| Failure | | | (−40.6%, | |
| | | | −1.2%) | |
| AMR | 10 (19.6%) | 18 (35.3%) | | |
| Graft Loss | 1 (2.0%) | 4 (7.8%) | | |
| Death | 1 (2.0%) | 1 (2.0%) | | |
| Lost to | 0 (0%) | 0 (0%) | | |
| Follow-Up | | | | |

To understand whether the patients reported through each set of reviews were consistently identified, a by-patient review of the diagnosis of each patient recorded as having acute AMR (both excluding and including Grade I acute AMR) was undertaken. The results were compared between the original Central biopsy, the reassessed Central biopsy and Local biopsy diagnoses.

The patients identified by the Central biopsy reassessment, in general, included the same patients who were identified by the original Central biopsy assessment. In addition, the majority of these patients were also identified in the Local biopsy assessment. This is an important observation because even though there was a level of discordance between Grades for the three different assessments, and although the Local pathologists identified a greater number of cases, the consistency between the three different evaluations is supportive of not only the reassessment, but overall is supportive of the outcome of the local biopsy assessment.

Tables 22 and 23 below provide the by-patient acute AMR diagnoses at the individual patient level for the original Central, reassessed Central and Local biopsies for the eculizumab and SOC arms, respectively. Each row shows a unique patient and thus, if a patient number is found in more than 1 column for a given row, it indicates agreement among the biopsy assessments.

### Eculizumab Arm

The reassessed Central biopsy data were consistent with the original Central biopsy data in the eculizumab arm; all patients identified initially were identified in the reassessment and one additional patient identified with the reassessment. This was true regardless of whether Grade I AMRs were excluded or included in the analysis.

When comparing the reassessed Central and Local data excluding Grade I, 2 patients were identified by Central pathology who were not identified by Local pathology (Patients 1 and 5). However, when Grade I cases were considered, Patient 1 was added. Thus, 4 of 6 and 5 of 6 patients diagnosed by the reassessed Central pathology were also diagnosed by the local pathology when excluding and including Grade I, respectively.

### Standard of Care Arm

The reassessed Central biopsy data were generally consistent with the original Central biopsy data in the SOC arm; 4 of 5 and 6 of 7 patients identified in the original biopsy evaluation were identified in the reassessment when considering those cases excluding and including Grade I, respectively. Five and 7 additional patients were identified with the reassessment of the original Central biopsies when excluding and including Grade I, respectively.

When comparing the reassessed Central and Local data excluding Grade I, 1 patient was identified by Central pathology that was not identified by Local pathology (Patient 20). When comparing the reassessed Central and Local data including Grade I, 2 patients were identified by Central pathology that were not identified by Local pathology (Patients 13 and 20). Altogether, 8 of 9 and 11 of 13 patients diagnosed by the reassessed Central pathology were also diagnosed by the local pathology when excluding and including Grade I, respectively.

### Summary of Central and Local Biopsy Assessments

The patients identified by the Central biopsy reassessment, in general, included the same patients who were identified by the original Central biopsy assessment. In addition, the majority of these patients were also identified in the Local biopsy assessment. This is an important observation because even though there was a level of discordance between Grades for the three different assessments, and although the Local pathologists identified a greater number of cases, the consistency between the three different evaluations is supportive of not only the reassessment, but overall is supportive of the outcome of the local biopsy assessment.

TABLE 22
By-Patient Comparison of the Acute AMR Endpoint Data: Central and
Reassessed Central Biopsy Data and Local Pathology Biopsy Data
Eculizumab (N = 51)

| Original Central | | Reassessed Central | | Local Pathology | |
|---|---|---|---|---|---|
| Biopsy Data | | Biopsy Data | | Biopsy Data | |
| Excluding | Including | Excluding | Including | Excluding | Including |
| Grade 1 | Grade 1 | Grade 1 | Grade 1 | Grade 1 | Grade 1 |
| 5 (9.8%) | 5 (9.8%) | 6 (11.8%) | 6 (11.8%) | 7 (13.7%) | 10 (19.6%) |
| 1 | 1 | 1 | 1 | | 1 |
| 2 | 2 | 2 | 2 | 2 | 2 |
| | | | | 3 | 3 |
| | | | | 4 | 4 |
| 5 | 5 | 5 | 5 | | |
| 6 | 6 | 6 | 6 | 6 | 6 |
| | | | | | 7 |
| 8 | 8 | 8 | 8 | 8 | 8 |
| | | | | 9 | 9 |
| | | | | | 10 |
| | | 11 | 11 | 11 | 11 |

Note:
The patient identification numbers have been simplified to maintain patient anonymity

TABLE 23
By-Patient Comparison of the Acute AMR Endpoint Data: Central and
Reassessed Central Biopsy Data and Local Pathology Biopsy Data
Standard of Care (N = 51)

| Original Central | | Reassessed Central | | Local Pathology | |
|---|---|---|---|---|---|
| Biopsy Data | | Biopsy Data | | Biopsy Data | |
| Excluding | Including | Excluding | Including | Excluding | Including |
| Grade 1 | Grade 1 | Grade 1 | Grade 1 | Grade 1 | Grade 1 |
| 5 (9.8%) | 7 (13.7%) | 9 (17.6%) | 13 (25.5%) | 12 | 18 |
| | | | | (23.5%) | (35.3%) |
| 12 | 12 | | | | 12 |
| | | | 13 | | |
| 14 | 14 | 14 | 14 | 14 | 14 |
| | 15 | 15 | 15 | 15 | 15 |
| | | | | | 16 |
| | | 17 | 17 | 17 | 17 |
| | | | | | 18 |
| | | | | 19 | 19 |
| 20 | 20 | 20 | 20 | | |
| | | | 21 | 21 | 21 |
| | | | 22 | | 22 |
| | 23 | 23 | 23 | 23 | 23 |
| 24 | 24 | 24 | 24 | 24 | 24 |
| | | | | 25 | 25 |
| | | | | 26 | 26 |
| 27 | 27 | 27 | 27 | 27 | 27 |
| | | | | | 28 |
| | | 29 | 29 | 29 | 29 |
| | | 30 | 30 | 30 | 30 |
| | | | 31 | | 31 |

Note:
The patient identification numbers have been simplified to maintain patient anonymity

As noted previously, one of the main goals of the biopsy reassessment was to better understand the discordance between the Local and Central pathology data and between the two Central pathologists.

Table 24 outlines the outcome of the original Local pathology compared to the reassessed Central pathology for both per-protocol and for-cause biopsies. There continued to be differences between Central and Local pathology outcomes; however, the simple kappa score, which is an assessment of agreement, increased from 0.225 (95% CI 0.111-0.338), considered fair or slight, to 0.496 (95% CI 0.374-0.618), considered moderate, in the current comparison.

TABLE 24
Pathology AMR Grading Results: Local versus Central
Pathology: Biopsy Reassessment

| | | Central | | Central | |
|---|---|---|---|---|---|
| | | Grade I | Central | Grade III | |
| Local | Central Clean | AMR | Grade II AMR | AMR | Total |
| Clean | 175 (72.6%) | 4 (1.7%) | 6 (2.5%) | 1 (0.4%) | 186 (77.2%) |
| Grade I acute | 13 (5.4%) | 6 (2.5%) | 4 (1.7%) | 0 | 23 (9.5%) |
| AMR | | | | | |
| Grade II acute | 11 (4.6%) | 3 (1.2%) | 14 (5.8%) | 0 | 28 (11.6% |
| AMR | | | | | |
| Grade III acute | 0 | 0 | 0 | 4 (1.7%) | 4 (1.7%) |
| AMR | | | | | |
| Total | 199 (82.6%) | 13 (5.4%) | 24 (10%) | 5 (2.1%) | 241 |

Total number of Biopsies are 241
Simple Kappa: 0.496
95% CI 0.374-0.618
Note:
the (percentages) above are the proportion of the total 241 biopsies categorically presented in each cell

Table 25 outlines the comparison between the individual Central pathologists when using the reassessed biopsy data. The level of agreement was higher in the reassessed biopsy data as evidenced by the improved simple kappa score of 0.457 (95% CI 0.335-0.580), versus the previous score of 0.372 (95% CI 0.220-0.523). However, there were 14 biopsies which were reported as clean by one of the Central pathologists but noted as Grade II or III for the other pathologist. The level of agreement between the Central pathologists based on kappa scores (0.457) was no better than the level of agreement between the overall Central Pathology and Local pathology (0.496).

TABLE 25
Central Pathology AMR Grading Results: Individual Primary
Pathologists: Biopsy Reassessment

| | | | Reader A: | | |
|---|---|---|---|---|---|
| Central Reader | Reader A: | Reader A: | Grade II | Reader A: | |
| B | Clean | Grade I AMR | AMR | Grade III AMR | Total |
| Clean | 191 (79.3%) | 0 | 1 (0.4%) | 0 | 192 (79.7%) |
| Grade I acute | 12 (5.0%) | 0 | 1 (0.4%) | 0 | 13 (5.4%) |
| AMR | | | | | |
| Grade II acute | 11 (4.6%) | 7 (2.9%) | 13 (5.4%) | 0 | 31 (12.9%) |
| AMR | | | | | |
| Grade III acute | 2 (0.8%) | 0 | 2 (0.8%) | 1 (0.4%) | 5 (2.1%) |
| AMR | | | | | |
| Total | 216 (89.6) | 7 (2.9%) | 17 (7.1%) | 1 (0.4%) | 241 |

Total number of Biopsies are 241
Simple Kappa: 0.457
95% CI 0.335-0.580
Note:
the (percentages) above are the proportion of the total 241 biopsies categorically presented in each cell

Table 26 gives a cross-tabulation of the “three” pathologists (each Local and the two Primary Central Pathologists) using the reassessed biopsy data. In this reassessment, out of the 241 biopsies, 191 were considered clean by at least one Central pathologist versus 208 in the original assessment. Comparing the Central pathologists, when a biopsy met the threshold for grade I, II, or III acute AMR by at least one pathologist, 14 of the 50 biopsies (28%) had an agreed diagnosis, compared with 6 of 33 (18%) in the original assessment. When considering the binary outcome of Clean plus Grade I, and Grade II plus Grade III, 28 of the 50 (56%) biopsy assessments were in agreement compared with 14 of 33 (42%) in the original assessment.

TABLE 26
Local and Central Acute AMR Grading Results (for-cause and per-
protocol): Comparison by Pathologist and Grade: Biopsy Reassessment

| | Central | | | |
|---|---|---|---|---|
| Local | Reader A | Central Reader B | | |
| Acute AMR | Acute AMR | Acute AMR | | |
| Grading Result | Grading Result | Grading Result | Frequency | Percent |
| Clean | Clean | Clean | 172 | 71.4 |
| Clean | Grade I | Clean | 4 | 1.7 |
| Clean | Grade II | Clean | 3 | 1.2 |
| Clean | Grade II | Grade I | 2 | 0.8 |
| Clean | Grade II | Grade II | 4 | 1.7 |
| Clean | Grade III | Grade III | 1 | 0.4 |
| Grade I | Clean | Clean | 11 | 4.6 |
| Grade I | Clean | Grade II | 1 | 0.4 |
| Grade I | Grade I | Clean | 5 | 2.1 |
| Grade I | Grade II | Clean | 4 | 1.7 |
| Grade I | Grade II | Grade I | 1 | 0.4 |
| Grade I | Grade II | Grade II | 1 | 0.4 |
| Grade II | Clean | Clean | 8 | 3.3 |
| Grade II | Grade I | Clean | 3 | 1.2 |
| Grade II | Grade I | Grade II | 1 | 0.4 |
| Grade II | Grade II | Clean | 4 | 1.7 |
| Grade II | Grade II | Grade I | 4 | 1.7 |
| Grade II | Grade II | Grade II | 8 | 3.3 |
| Grade III | Grade III | Clean | 2 | 0.8 |
| Grade III | Grade III | Grade II | 2 | 0.8 |

Note:
Bolded cells denote complete agreement between pathologists

### CONCLUSIONS

The primary endpoint was based on a Central pathology assessment of for-cause biopsies. The level of discordance noted in the original assessment raised a serious question about whether the outcome of the trial based on this assessment was accurate. Consultations with experts in the transplantation of highly sensitized patients led to a reassessment of a subset of biopsies by the Central pathologists, in which the pathologists had access to certain clinical information that was available to Local pathologists. Importantly, Central pathologists remained blinded to the treatment regimen during the reassessment. In addition, the Central pathologists followed the strict criteria of the protocol with regard to the Banff criteria, which were not followed in the original assessments. The outcome of the reassessments resulted in a greater level of agreement between the Central pathologists, as well as a greater level of agreement between the Local and Central pathology. However, the agreement between the individual Central pathologists was no better than the level of agreement between the overall Central Pathology and Local pathology. This outcome calls into question the added value of using Central pathology over the Local pathology especially given that treatment decisions are made based on local pathology.

An additional outcome of the reassessment was a greater difference noted between the eculizumab and SOC groups for treatment failure due to additional acute AMR diagnoses resulting from the reassessment. Although this difference in the primary endpoint did not reach statistical significance, the inclusion of Grade I biopsies in both Local and the reassessed Central pathology results, based on input from experts in acute AMR, did produce a statistically significant benefit favoring eculizumab over SOC.

It is not possible to determine whether the additional cases of acute AMR diagnosed in the reassessment were due to the ability of the Central pathologists to review the clinical information, or due to their strictly following the protocol-required Banff criteria, given that not all of the details of the criteria were captured in the original assessment. When considering all three biopsy assessments, the patients identified in the original Central biopsy assessment continued to be identified in the biopsy reassessment as well as the Local biopsy assessment. Importantly, the totality of the data indicates that eculizumab had a meaningful effect in the prevention of acute AMR, especially when all Grades I, II, and III were considered. Although Grade I cases were not included in the primary endpoint for this trial, at the time this study was designed, there was very little clinical experience that supported the understanding of the histologic features of acute AMR. Given our current understanding of this early acute lesion, such cases, which were diagnosed based on for-cause biopsies, are relevant to patients outcomes and are thought to represent early identification of lesions that, if left untreated, would be expected to progress to higher grades of acute AMR.

### Summary of Sequence Listing

SEQ ID NO: 1

GYIFSNYWIQ

SEQ ID NO: 2

EILPGSGSTEYTENFKD

SEQ ID NO: 3

YFFGS SPNWYFDV

SEQ ID NO: 4

GASENIYGALN

SEQ ID NO: 5

GATNLAD

SEQ ID NO: 6

QNVLNTPLT

SEQ ID NO: 7

QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSS

SEQ ID NO: 8

DIQMTQSPSSLSASVGDRVTITCGASENIYGALNWYQQKPGKAPKWYGA
TNLADGVPSRFSGSGSGTDFTLTISSLQPEDFATYYCQNVLNTPLTFGQG
TKVEIK

SEQ ID NO: 9

ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGK

SEQ ID NO: 10

QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVPSSNF
GTQTYTCNVDFIKPSNTKVDKTVERKCCVECPPCPAPPVAGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPS SIEKTISKAKGQPRE
PQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSL
GK

SEQ ID NO: 11

DIQMTQSPSSLSASVGDRVTITCGASENIYGALNWYQQKPGKAPKWYG
ATNLADGVPSRFSGSGSGTDFTLTISSLQPEDFATYYCQNVLNTPLTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

SEQ ID NO: 12

QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSS

SEQ ID NO: 13

ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVLHEALHSHYTQKSLSLSLGK

SEQ ID NO: 14

QVQLVQSGAEVKKPGASVKVSCKASGHIFSNYWIQWVRQAPGQGLEWM
GEILPGSGHTEYTENFKDRVTMTRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVPSSNF
GTQTYTCNVDFIKPSNTKVDKTVERKCCVECPPCPAPPVAGPSVFLFPPK
PKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPP
VLDSDGSFFLYSRLTVDKSRWQEGNVFSCSVLHEALHSHYTQKSLSLSLG
K

SEQ ID NO: 15

ASTKGPSVFPLAPCSRSTSESTAALGCLVKDYFPEPVTVSWNSGALTSGV
HTFPAVLQSSGLYSLSSVVTVTSSNFGTQTYTCNVDHKPSNTKVDKTVER
KCCVECPPCPAPPVAGPSVFLFPPKPKDTLYITREPEVTCVVVDVSHEDP
EVQFNWYVDGMEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK

SEQ ID NO: 16

QVQLVQSGAEVKKPGASVKVSCKASGYIFSNYWIQWVRQAPGQGLEWM
GEILPGSGSTEYTENFKDRVTMTRDTSTSTVYMELSSLRSEDTAVYYCAR
YFFGSSPNWYFDVWGQGTLVTVSSASTKGPSVFPLAPCSRSTSESTAALG
CLVKDYFPEPVTVSWNSGALTSGVHTFPAVLQSSGLYSLSSVVTVTSSNF
GTQTYTCNVDHKPSNTKVDKTVERKCCVECPPCPAPPVAGPSVFLFPPKP
KDTLYITREPEVTCVVVDVSHEDPEVQFNWYVDGMEVHNAKTKPREEQ
FNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLS
PGK

SEQ ID NO: 17

GASENIYHALN

SEQ ID NO: 18

EILPGSGHTEYTENFKD

SEQ ID NO: 19

GHIFSNYWIQ

SEQ ID NO: 20

QVQLVQSGAEVKKPGASVKVSCKASGHIFSNYWIQWVRQAPGQGLEW
MGEILPGSGHTEYTENFKDRVTMTRDTSTSTVYMELSSLRSEDTAVYYC
ARYFFGSSPNWYFDVWGQGTLVTVSSASTKGPSVFPLAPCSRSTSESTAA
LGCLVKDYFPEPVTVSWNSGALTSGVHTFPAVLQSSGLYSLSSVVTVPSS
NFGTQTYTCNVDFIKPSNTKVDKTVERKCCVECPPCPAPPVAGPSVFLFP
PKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLS
LGK

SEQ ID NO: 21

SYAIS

SEQ ID NO: 22

GIGPFFGTANYAQKFQG

SEQ ID NO: 23

DTPYFDY

SEQ ID NO: 24

SGDSIPNYYVY

SEQ ID NO: 25

DDSNRPS

SEQ ID NO: 26

QSFDSSLNAEV

SEQ ID NO: 27

QVQLVQSGAEVKKPGSSVKVSCKASGGTFSSYAISVWRQAPGQGLEWMGG
IGPFFGTANY
AQKFQGRVTITADESTSTAYMELSSLRSEDTAVYYCARDTPYFD
YWGQGTLVTVSS

SEQ ID NO: 28

DIELTQPPSVSVAPGQTARISCSGDSIPNYYVYWYQQKPGQAPVLVIYDD
SNRPSGIPERFSGSNSGNTATLTISGTQAEDEADYYCQSFDSSLNAEVFG
GGTK LTVL

SEQ ID NO: 29

NYIS

SEQ ID NO: 30

IIDPDDSYTEYSPSFQG

SEQ ID NO: 31

YEYGGFDI

SEQ ID NO: 32

SGDNIGNSYVH

SEQ ID NO: 33

KDNDRPS

SEQ ID NO: 34

GTYDIESYV

SEQ ID NO: 35

EVQLVQSGAEVKKPGESLKISCKGSGYSFTNYISWVRQMPGKGLEWMGII
DPDDSYTEYSPSFQGQVTI SADKSISTAYLQWSSLKASDTAMYYCARYE
YGGFDI WGQGTLVTVSS

SEQ ID NO: 36

SYELTQPPSVSVAPGQTARISCSGDNIGNSYVHWYQQKPGQAPVLVIYKD
NDRPSGIPERFSG
SNSGNT ATLTISGTQAEDEADYYCGTYDIESYVFGGGTKLTV L

SEQ ID NO: 37

QVQLVESGGGLVQPGRSLRLSCAASGFTVHSSYYMAWVRQAPGKGLEWVG
AIFTGSGAEYKAEWAKGRVTISKDTSKNQVVLTMTNMDPVDTATYYCASD
AGYDYPTHAMHYWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELRRGPKVFLFP
PKPKDTLMISRTPEVTCVVVDVSHEDPEVKFNWYVDGVEVHNAKTKPREE
QYNSTYRVVSVLTVLHQDWLNGKEYKCKVSNKGLPSSIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVLHEALHAHYTRKELSLS
P

SEQ ID NO: 38

DIQMTQSPSSLSASVGDRVTITCRASQGISSSLAWYQQKPGKAPKLLIYG
ASETESGVPSRFSGSGSGTDFTLTISSLQPEDFATYYCQNTKVGSSYGNT
FGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVT
HQGLSSPVTKSFNRGEC

1. A method of reducing antibody mediated rejection (AMR) in a human kidney transplant recipient, comprising administering a therapeutically effective amount of an anti-C5 antibody, or antigen-binding fragment thereof, to the recipient in a phased dosing schedule following reperfusion of a kidney allograft, wherein the recipient is sensitized to a human living donor and wherein the recipient receives about two or more weeks of desensitization therapy prior to transplantation.

2. The method of claim 1, wherein the recipient receives about two, three, or four weeks of desensitization therapy prior to transplantation.

3. (canceled)

4. (canceled)

5. The method of claim 1, wherein the phased dosing schedule comprises about a 1200 mg dose of antibody administered about 1 hour prior to kidney allograft reperfusion; followed by about a 900 mg dose administered at about day 1, and about day 7, and about day 14, and about day 21, and about day 28 post transplantation; and about a 1200 mg dose administered at about week 5; and about week 7, and about week 9 post transplantation.

6. The method of claim 1, wherein the recipient's medical history includes prior exposure to HLA, wherein the prior exposure to HLA includes one or more of prior solid organ or tissue allograft, pregnancy, blood transfusion, or prior exposure to the specific donor's HLA.

7. (canceled)

8. The method of claim 1, wherein the desensitization therapy comprises intravenous immunoglobulin treatment (IVIg) or plasmapheresis treatment.

9. (canceled)

10. The method of claim 1, wherein the recipient experiences one or more of the following:
(a) reduced AMR compared to standard of care (SOC) and/or wherein the recipient experiences reduced graft loss compared to SOC;
(b) a clinically meaningful low level of circulating anti-donor specific antibodies during about the first 9 weeks post transplantation compared to the absence of therapy with the antibody, or antigen binding fragment thereof;
(c) a clinically meaningful low level of circulating anti-donor specific antibodies during about the first 12 months post transplantation, compared to the absence of therapy with the antibody, or antigen binding fragment thereof;
(d) a clinically meaningful low level of morphologic evidence of acute tissue injury during about the first 9 weeks post transplantation, compared to the absence of therapy with the antibody, or antigen binding fragment thereof;
(e) a clinically meaningful low level of morphologic evidence of acute tissue injury during about the first 12 months post transplantation, compared to the absence of therapy with the antibody, or antigen binding fragment thereof;
(f) a clinically meaningful increase in graft survival at about week 9 post transplantation, compared to the absence of therapy with the antibody, or antigen binding fragment thereof;
(g) a clinically meaningful increase in graft survival at about month 12 post transplantation, compared to the absence of therapy with the antibody, or antigen binding fragment thereof;
(h) increased survival at about 9-weeks post transplantation compared to the absence of therapy with the antibody or antigen binding fragment thereof;
(i) increased survival at about 12-months post transplantation compared to the absence of therapy with the antibody or antigen binding fragment thereof;
(j) increased survival at about 36-months post transplantation compared to the absence of therapy with the antibody, or antigen binding fragment thereof;
(k) clinically meaningful low histological evidence of antibody mediated rejection during about the first 9 weeks post transplantation, compared to the absence of therapy with the antibody, or antigen binding fragment thereof;
(l) clinically meaningful low histological evidence of antibody mediated rejection during about the first 12 months post transplantation, compared to the absence of therapy with the antibody, or antigen binding fragment thereof;
(m) clinically meaningful low pathological changes, including chronic AMR, on biopsies during about the first 9 weeks post transplantation, compared to the absence of therapy with the antibody, or antigen binding fragment thereof;
(n) clinically meaningful low pathological changes, including chronic AMR, on biopsies during about the first 12 months post transplantation compared to the absence of therapy with the antibody, or antigen binding fragment thereof;
(o) a reduced need for plasmapheresis treatments during about the first 9 weeks post transplantation compared to the absence of therapy with the antibody, or antigen binding fragment thereof;
(p) a reduced need for plasmapheresis treatments during about the first 12 months post transplantation compared to the absence of therapy with the antibody, or antigen binding fragment thereof;
(g) clinically meaningful reduced delayed graft function post transplantation compared to the absence of therapy with the antibody, or antigen binding fragment thereof;
(r) a clinically meaningful reduction in need for dialysis during about the first 9 weeks post transplantation, compared to the absence of therapy with the antibody, or antigen binding fragment thereof;
(s) a clinically meaningful reduction in need of dialysis during about the first 12 months post transplantation, compared to the absence of therapy with the antibody, or antigen binding fragment thereof;
(t) stable renal function during about the first 9 weeks post transplantation compared to the absence of therapy with the antibody, or antigen binding fragment thereof; and/or
(u) stable renal function during about the first 12 months post transplantation compared to the absence of therapy with the antibody, or antigen binding fragment thereof.

11-30. (canceled)

31. A method of reducing antibody mediated rejection (AMR) in a human kidney transplant recipient, comprising administering a therapeutically effective amount of an anti-C5 antibody, or antigen-binding fragment thereof, to the recipient in a phased dosing schedule following reperfusion of a kidney allograft, wherein the recipient is sensitized to a human living donor and wherein the recipient receives about two or more weeks of desensitization therapy prior to transplantation,
wherein the recipient experiences during about the first 9 weeks post transplantation, during about the first 12 months post transplantation, and/or during about the first 36 months post transplantation, one or more of: clinically meaningful low level of circulating anti-donor specific antibodies, clinically meaningful low level of morphologic evidence of acute tissue injury, clinically meaningful low histological evidence of antibody mediated rejection, increased greater survival, or increased survival, clinically meaningful low histological evidence of antibody mediated rejection, clinically meaningful low pathological changes, including chronic AMR, on biopsies, reduced need for plasmapheresis treatments, clinically significant reduction in need of dialysis, compared to the absence of therapy with the antibody or antigen binding fragment thereof.

32. The method of claim 1, wherein the anti-C5 antibody or an antigen-binding fragment thereof is administered subcutaneously or through intravenous infusion.

33. (canceled)

34. The method of claim 1, wherein the recipient's plasma levels of anti-C5 antibody, or an antigen binding fragment thereof, is maintained at about 50 to about 100 μg/mL for about the first week post transplantation.

35. The method of claim 1, wherein the recipient's plasma levels of anti-C5 antibody, or an antigen binding fragment thereof, is maintained at about 50 to about 100 μg/mL for about the first 9 weeks post transplantation.

36. The method of claim 1, further comprising administering to the recipient one or more immunosuppressive drug selected from the group consisting of tacrolimus, mycophenolate mofetil, and prednisone.

37. The method of claim 1, wherein the anti-C5 antibody is eculizumab.

38. The method of claim 1, wherein the anti-C5 antibody is BNJ441.

39. The method of claim 1, wherein the anti-C5 antibody is BNJ421.

40. The method of claim 1, wherein the anti-C5 antibody, or an antigen binding fragment thereof, comprises CDR1, CDR2, and CDR3 heavy chain sequences as set forth in SEQ ID NOs: 1, 2, and 3, respectively, and CDR1, CDR2, and CDR3 light chain sequences as set forth in SEQ ID NOs: 4, 5, and 6, respectively.

41. The method of claim 1, wherein the anti-C5 antibody, or antigen binding fragment thereof, comprises the V_{H} domain having the sequence set forth in SEQ ID NO:7, and the V_{L} domain having the sequence set forth in SEQ ID NO:8, respectively.

42. The method of claim 1, wherein the anti-C5 antibody, or an antigen binding fragment thereof, comprises a heavy chain constant region having the amino acid sequences set forth in SEQ ID NO: 9.

43. The method of claim 1, wherein the anti-C5 antibody, or an antigen binding fragment thereof, comprises the entire heavy chain and light chains having the amino acid sequences set forth in SEQ ID NO: 10 and SEQ ID NO: 11, respectively.

44. The method of claim 1, wherein the anti-C5 antibody, or an antigen binding fragment thereof, comprises CDR1, CDR2, and CDR3 heavy chain sequences as set forth in SEQ ID NOs:19, 18, and 3, respectively, and CDR1, CDR2, and CDR3 light chain sequences as set forth in SEQ ID NOs: 4, 5, and 6, respectively.

45. The method of claim 1, wherein the anti-C5 antibody, or an antigen binding fragment thereof, comprises the V_{H} domain having the sequence set forth in SEQ ID NO:12, and the V_{L} domain having the sequence set forth in SEQ ID NO:8, respectively.

46. The method of claim 1, wherein the anti-C5 antibody, or antigen binding fragment thereof, comprises a heavy chain constant region having the amino acid sequences set forth in SEQ ID NO: 13.

47. The method of claim 1, wherein the anti-C5 antibody, or an antigen binding fragment thereof, comprises the entire heavy chain and light chains having the amino acid sequences set forth in SEQ ID NO: 14 and SEQ ID NO: 11, respectively.


SEQUENCE LISTING (38 sequences)

SEQ ID NO: 1 - protein, 10 aa, Artificial Sequence
  source: /note=“Description of Artificial Sequence Synthetic peptide”
GYIFSNYWIQ

SEQ ID NO: 2 - protein, 17 aa, Artificial Sequence
  source: /note=“Description of Artificial Sequence Synthetic peptide”
EILPGSGSTEYTENFKD

SEQ ID NO: 3 - protein, 13 aa, Artificial Sequence
  source: /note=“Description of Artificial Sequence Synthetic peptide”
YFFGSSPNWYFDV

SEQ ID NO: 4 - protein, 11 aa, Artificial Sequence
  source: /note=“Description of Artificial Sequence Synthetic peptide”
GASENIYGALN

SEQ ID NO: 5 - protein, 7 aa, Artificial Sequence
  source: /note=“Description of Artificial Sequence Synthetic peptide”
GATNLAD

SEQ ID NO: 6 - protein, 9 aa, Artificial Sequence
  source: /note=“Description of Artificial Sequence Synthetic peptide”
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  source: /note=“Description of Artificial Sequence Synthetic polypeptide”
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  source: /note=“Description of Artificial Sequence Synthetic peptide”
GASENIYHALN

SEQ ID NO: 18 - protein, 17 aa, Artificial Sequence
  source: /note=“Description of Artificial Sequence Synthetic peptide”
EILPGSGHTEYTENFKD

SEQ ID NO: 19 - protein, 10 aa, Artificial Sequence
  source: /note=“Description of Artificial Sequence Synthetic peptide”
GHIFSNYWIQ

SEQ ID NO: 20 - protein, 448 aa, Artificial Sequence
  source: /note=“Description of Artificial Sequence Synthetic polypeptide”
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  source: /note=“Description of Artificial Sequence Synthetic peptide”
SYAIS

SEQ ID NO: 22 - protein, 17 aa, Artificial Sequence
  source: /note=“Description of Artificial Sequence Synthetic peptide”
GIGPFFGTANYAQKFQG

SEQ ID NO: 23 - protein, 7 aa, Artificial Sequence
  source: /note=“Description of Artificial Sequence Synthetic peptide”
DTPYFDY

SEQ ID NO: 24 - protein, 11 aa, Artificial Sequence
  source: /note=“Description of Artificial Sequence Synthetic peptide”
SGDSIPNYYVY

SEQ ID NO: 25 - protein, 7 aa, Artificial Sequence
  source: /note=“Description of Artificial Sequence Synthetic peptide”
DDSNRPS

SEQ ID NO: 26 - protein, 11 aa, Artificial Sequence
  source: /note=“Description of Artificial Sequence Synthetic peptide”
QSFDSSLNAEV

SEQ ID NO: 27 - protein, 116 aa, Artificial Sequence
  source: /note=“Description of Artificial Sequence Synthetic polypeptide”
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  source: /note=“Description of Artificial Sequence Synthetic polypeptide”
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  source: /note=“Description of Artificial Sequence Synthetic peptide”
NYIS

SEQ ID NO: 30 - protein, 17 aa, Artificial Sequence
  source: /note=“Description of Artificial Sequence Synthetic peptide”
IIDPDDSYTEYSPSFQG

SEQ ID NO: 31 - protein, 8 aa, Artificial Sequence
  source: /note=“Description of Artificial Sequence Synthetic peptide”
YEYGGFDI

SEQ ID NO: 32 - protein, 11 aa, Artificial Sequence
  source: /note=“Description of Artificial Sequence Synthetic peptide”
SGDNIGNSYVH

SEQ ID NO: 33 - protein, 7 aa, Artificial Sequence
  source: /note=“Description of Artificial Sequence Synthetic peptide”
KDNDRPS

SEQ ID NO: 34 - protein, 9 aa, Artificial Sequence
  source: /note=“Description of Artificial Sequence Synthetic peptide”
GTYDIESYV

SEQ ID NO: 35 - protein, 116 aa, Artificial Sequence
  source: /note=“Description of Artificial Sequence Synthetic polypeptide”
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  source: /note=“Description of Artificial Sequence Synthetic polypeptide”
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 451 aa, Artificial Sequence
  source: /note=“Description of Artificial Sequence Synthetic polypeptide”
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 38 - protein, 217 aa, Artificial Sequence
  source: /note=“Description of Artificial Sequence Synthetic polypeptide”
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC